(19) United States
(12) Patent Application Publication
(10) Pub. No.: US 2025/0108131 A1
(43) Pub. Date: Apr. 3, 2025

(54) TRIGGER NUCLEIC ACIDS AND RNA-BINDING PROTEINS FOR UPREGULATING GENE EXPRESSION

(71) Applicant: Whitehead Institute for Biomedical Research, Cambridge, MA (US)

(72) Inventors: Jonathan Weissman, San Francisco, CA (US); Mohamed El-Brolosy, Cambridge, MA (US); André Fischer, Vienna (AT); Atharv Vivek Oak, Baltimore, MD (US)

(73) Assignee: Whitehead Institute for Biomedical Research, Cambridge, MA (US)

(21) Appl. No.: 18/899,814

(22) Filed: Sep. 27, 2024

Related U.S. Application Data
(60) Provisional application No. 63/669,032, filed on Jul. 9, 2024.
(60) Provisional application No. 63/586,863, filed on Sep. 29, 2023.

(51) Int. Cl. A61K 48/00; A61K 38/17; C07K 14/47; C12N 9/22; C12N 15/11; C12N 15/113; C12Q 1/6804; C12Q 1/6869; G01N 33/50

(52) U.S. Cl. CPC A61K 48/005; A61K 38/1709; C07K 14/47; C12N 9/22; C12N 15/111; C12N 15/113; C12Q 1/6804; C12Q 1/6869; G01N 33/5023; C07K 2319/09; C07K 2319/85; C12N 2310/11; C12N 2310/20

(57) ABSTRACT

The present disclosure, at least in part, relates to compositions (e.g., engineered nucleic acids and engineered proteins) and methods for increasing gene expression. The engineered proteins include RNA-binding proteins (e.g., RNA-binding proteins that comprise a Interleukin Enhancer Binding Factor 3 (ILF3) sequence, a Cas sequence, or a combination thereof). In some aspects, the disclosure provides methods of identifying engineered nucleic acids that are shorter in length than a gene of interest to induce expression of the gene of interest and also provides RNA-binding proteins for inducing gene expression.

### RELATED APPLICATIONS

This application claims priority under 35 U.S.C. § 119(e) to U.S. Provisional application, U.S. Ser. No. 63/586,863, filed on Sep. 29, 2023 and to U.S. Provisional application, U.S. Ser. No. 63/669,032, filed on Jul. 9, 2024, each of which is incorporated herein by reference.

### REFERENCE TO AN ELECTRONIC SEQUENCE LISTING

The contents of the electronic sequence listing (W057170063US02-SEQ-FL.xml; Size: 134,044 bytes; and Date of Creation: Sep. 20, 2024) is herein incorporated by reference in its entirety.

### BACKGROUND

Dysregulation of gene expression is a hallmark of numerous diseases, including genetic diseases and cancer. For example, some diseases are characterized by overexpression of one or more genes that results in an aberrant increase in the activity of one or more proteins encoded by the one or more genes. In contrast, some diseases are characterized by an aberrant decrease in expression of one or more genes that downregulates the activity of one or more proteins encoded by the one or more genes. While target-based strategies for downregulating gene expression have been well-characterized, options for increasing gene expression and protein activity have been relatively limited.

### SUMMARY OF THE INVENTION

Although the development of targeted therapeutics has increased the arsenal of drugs against numerous genetic disorders, existing therapies are largely focused on downregulating gene expression. However, diseases including haploinsufficiency disorders and autosomal recessive disorders are characterized by a decrease in expression of one or more functional proteins. Existing overexpression systems often inundate cells with non-physiological levels of gene expression, and it is often difficult to deliver large nucleic vectors encoding a protein of interest to cells. Described herein, in some embodiments, are compositions, kits, systems, and methods for increasing expression of one or more genes of interest, e.g., via an engineered nucleic acid alone or in combination with a ribonucleic acid (RNA)-binding protein, to address many of these limitations.

In some aspects, the disclosure is based on the findings that vectors capable of inducing RNA decay may be used to identify oligonucleotides that are useful in increasing expression of one or more genes of interest, and that the RNA-binding protein, Interleukin Enhancer Binding Factor 3 (ILF3), or fragments thereof may be used to increase gene expression. In some embodiments, the identified oligonucleotides or fragments thereof alone are sufficient to upregulate expression of a gene of interest. In some instances, the identified oligonucleotides or fragments thereof may deactivate an antisense oligonucleotide of the gene of interest by downregulating activity of the antisense transcript. As a non-limiting example, deactivation of an antisense transcript may result from preventing the antisense transcript from binding to the mRNA or promoting degradation of the transcript. In some embodiments, the identified oligonucleotides or fragments thereof may be used to target an RNA-binding protein to an antisense transcript of the gene of interest. In some embodiments, the identified oligonucleotides or fragments thereof may be used in combination with an RNA-binding protein to target the RNA binding protein to an antisense transcript of a paralog of the gene of interest.

In some instances, the RNA-binding protein comprises an Interleukin Enhancer Binding Factor 3 (ILF3) sequence and/or the sequence of an RNA-targeting Cas protein. In some embodiments, the RNA-targeting Cas protein does not comprise nuclease activity toward a target RNA. In some embodiments, the ILF3 sequence recruits transcription factors (TFs) and chromatin remodelers (CRs) to promote gene expression.

Without wishing to be bound by any particular theory, in some embodiments, the methods disclosed herein increase gene expression by targeting RNA, which may be advantageous over existing CRISPR-based methods that rely on targeting the DNA encoding the RNA and fusing Cas proteins to transcriptional activators (e.g., CRISPR-mediated transcriptional activation (CRISPRa)) because, for example, targeting the DNA requires targeting a very narrow window around the gene's transcription start site which limits the number of guide sequences that may be designed. In some embodiments, targeting RNAs allows for a broader window for targeting and designing guide RNAs, e.g., the entire length of the RNA transcript may be used to target and design guide RNAs. In some embodiments, an engineered nucleic acid disclosed herein targets antisense RNAs, which may allow for tissue specific upregulation of gene expression because antisense RNAs are often tissue-specific unlike methods that target DNA.

Aspects of the present disclosure provide a non-naturally occurring protein, wherein the non-naturally occurring protein comprises an ILF3 sequence, wherein the ILF3 sequence comprises a deletion of one or more of the following domains relative to a wild-type ILF3 sequence: GQSY-repeat motif, double-stranded RNA-binding domain 1 (dsRBD1) domain, double-stranded RNA-binding domain 2 (dsRBD2) domain, a nuclear localization signal (NLS), a RGG-repeat motif, an NES domain, a DZF domain, and/or a NVKQ motif (NVKQ), optionally wherein the ILF3 sequence comprises a deletion of one or more of the following domains relative to a wild-type ILF3 sequence: an NES domain, a DZF domain, and/or a NVKQ motif (NVKQ).

Further aspects of the present disclosure provide a non-naturally occurring protein, wherein the non-naturally occurring protein comprises an ILF3 sequence, wherein the ILF3 sequence comprises: a double-stranded RNA-binding domain 1 (dsRBD1) domain, double-stranded RNA-binding domain 2 (dsRBD2) domain, a nuclear localization signal (NLS), GQSY-repeat domain, and a RGG-repeat motif.

Further aspects of the present disclosure provide a fusion protein comprising: an ILF3 sequence linked to an RNA-targeting Cas protein, wherein the nuclease activity of the RNA-targeting Cas protein toward target RNA is inactive, optionally wherein the ILF3 sequence is any of the ILF3 sequences described herein.

In some embodiments, the RNA-targeting Cas protein is a Type VI Cas protein. In some embodiments, the Type VI Cas protein is a Cas13 protein. In some embodiments, the Cas13 protein is a Cas13a, Cas13b, Cas13C, Cas13d, or Cas13bt protein. In some embodiments, the RNA-targeting Cas protein is a Type III Cas protein. In some embodiments, the Type III Cas protein is a Csm protein or a Cmr protein. In some embodiments, the RNA-targeting Cas protein is a Cas7-11 Cas protein. In some embodiments, the RNA-targeting Cas protein is linked to a nuclear localization signal sequence. In some embodiments, the ILF3 sequence comprises an amino acid sequence that is at least 90% identical to one or more of SEQ ID NOs: 1-14, 61, and 69. In some embodiments, the RNA-targeting Cas protein comprises an amino acid sequence that is at least 90% identical to SEQ ID NO: 63. In some embodiments, the RNA-targeting Cas protein does not comprise SEQ ID NO: 64 and/or does not comprise SEQ ID NO: 65, optionally wherein the Cas protein comprises SEQ ID NO: 80 and/or SEQ ID NO: 81. In some embodiments, the fusion protein comprises an amino acid sequence that is at least 90% identical to any one of SEQ ID NOs: 1-14, 61-63, 66-69, and 80-81.

Further aspects of the present disclosure provide an engineered nucleic acid encoding any of the non-naturally occurring proteins or fusion proteins described herein.

In some embodiments, the engineered nucleic acid is an expression vector. In some embodiments, the engineered nucleic acid is a viral vector.

Further aspects of the present disclosure provide a virus comprising any of the engineered nucleic acids described herein.

Further aspects of the present disclosure provide a lipid nanoparticle that encapsulates any of the non-naturally occurring proteins or fusion proteins described herein or any of the engineered nucleic acids described herein.

Further aspects of the present disclosure provide a composition comprising any of the non-naturally occurring proteins or fusion proteins described herein, any of the engineered nucleic acids described herein, any of the viruses described herein, or any of the lipid nanoparticles described herein.

In some embodiments, the composition further comprising a guide RNA targeting a transcript of a gene of interest. In some embodiments, the transcript is an antisense transcript. In some embodiments, the transcript is a sense RNA transcript.

In some embodiments the guide RNA targets ACTG1, ACTG2, CDK9, REL, BDNF, or SOX9.

In some embodiments, the guide RNA is 19-23 nucleotides in length.

In some embodiments, the guide RNA comprises a nucleotide sequence that is at least 90% identical to any one of SEQ ID NOs: 15-34.

Further aspects of the present disclosure provide a composition comprising a RNA-targeting Cas protein, wherein the RNA-targeting Cas protein does not comprise nuclease activity toward a transcript of a gene of interest and a guide RNA targeting the transcript of a gene of interest.

In some embodiments, the RNA-targeting Cas protein is a Type VI Cas protein. In some embodiments, the Type VI Cas protein is a Cas13 protein. In some embodiments, the Cas13 protein is a Cas13a, Cas13b, Cas13C, Cas13d, or Cas13bt protein. In some embodiments, the RNA-targeting Cas protein is a Type III Cas protein. In some embodiments, the Type III Cas protein is a Csm protein or a Cmr protein. In some embodiments, the RNA-targeting Cas protein is a Cas7-11 Cas protein. In some embodiments, the guide RNA comprises a nucleic acid sequence that is at least 90% identical to any one of SEQ ID NOs: 15-34. In some embodiments, the transcript is an antisense transcript. In some embodiments, the transcript is a sense transcript. In some embodiments, any of the compositions or lipid nanoparticles described herein comprising any of the non-naturally occurring protein or fusion protein described herein.

Further aspects of the present disclosure provide a composition comprising a lipid nanoparticle that encapsulates a RNA-targeting Cas protein, wherein the RNA-targeting Cas protein does not comprise nuclease activity toward a transcript of a gene of interest and a guide RNA targeting the transcript of a gene of interest.

In some embodiments, the RNA-targeting Cas protein is a Type VI Cas protein. In some embodiments, the Type VI Cas protein is a Cas13 protein. In some embodiments, the Cas13 protein is a Cas13a, Cas13b, Cas13C, Cas13d, or Cas13bt protein. In some embodiments, the RNA-targeting Cas protein is a Type III Cas protein. In some embodiments, the Type III Cas protein is a Csm protein or a Cmr protein. In some embodiments, the RNA-targeting Cas protein is a Cas7-11 Cas protein. In some embodiments, the guide RNA comprises a nucleic acid sequence that is at least 90% identical to any one of SEQ ID NOs: 15-34. In some embodiments, the transcript is an antisense transcript. In some embodiments, the transcript is a sense transcript. In some embodiments, any of the compositions or lipid nanoparticles described herein comprising any of the non-naturally occurring protein or fusion protein described herein.

Further aspects of the present disclosure provide a lipid nanoparticle that encapsulates a RNA-targeting Cas protein, wherein the RNA-targeting Cas protein does not comprise nuclease activity toward a transcript of a gene of interest and a guide RNA targeting the transcript of a gene of interest.

In some embodiments, the RNA-targeting Cas protein is a Type VI Cas protein. In some embodiments, the Type VI Cas protein is a Cas13 protein. In some embodiments, the Cas13 protein is a Cas13a, Cas13b, Cas13C, Cas13d, or Cas13bt protein. In some embodiments, the RNA-targeting Cas protein is a Type III Cas protein. In some embodiments, the Type III Cas protein is a Csm protein or a Cmr protein. In some embodiments, the RNA-targeting Cas protein is a Cas7-11 Cas protein. In some embodiments, the guide RNA comprises a nucleic acid sequence that is at least 90% identical to any one of SEQ ID NOs: 15-34. In some embodiments, the transcript is an antisense transcript. In some embodiments, the transcript is a sense transcript. In some embodiments, any of the compositions or lipid nanoparticles described herein comprising any of the non-naturally occurring protein or fusion protein described herein.

Further aspects of the present disclosure provide a method of identifying one or more oligonucleotides that are capable of upregulating expression of a gene of interest comprising:
(a) contacting cells with a population of expression vectors, wherein the cells are eukaryotic cells and each expression vector (i) is capable of inducing RNA decay and (ii) encodes an oligonucleotide that is less than 300 nucleotides in length operably linked to a promoter;
(b) identifying a subset of the cells as having increased expression of the gene of interest as compared to control cells; and
(c) detecting one or more oligonucleotides in the subset of the cells, thereby identifying one or more oligonucleotides that are capable of upregulating expression of a gene of interest.

In some embodiments, each expression vector comprises in the following order:
(i) a first stop codon following the oligonucleotide;
(ii) an exon of a second gene;
(iii) an intron of the second gene;
(iv) a second exon of the second gene; and
(v) a second stop codon following the second exon of the second gene.
   In some embodiments, wherein each expression vector comprises a plurality of sets of (ii)-(v).

In some embodiments, each expression vector encodes a self-complementary sequence downstream of the oligonucleotide.

In some embodiments, each expression vector encodes two or more contiguous lysine residues downstream of the oligonucleotide sequence and wherein each expression vector does not include a stop codon between the oligonucleotide sequence and the sequence encoding the two or more contiguous lysine residues. In some embodiments, the two or more contiguous lysine residues are encoded by a nucleic acid sequence comprising the sequence AAA and/or AAG.

In some embodiments, the oligonucleotide is a segment of a gene that is a paralog of the gene of interest.

In some embodiments, the oligonucleotide is a segment of the gene of interest.

In some embodiments, the control cells are cells that do not comprise an expression vector encoding one or more of the oligonucleotides.

In some embodiments, the eukaryotic cell is a mouse cell. In some embodiments, the eukaryotic cell is a human cell. In some embodiments, a method described herein further comprising administering one or more of the expression vectors that is capable of inducing RNA decay that encode an oligonucleotide identified as being capable of upregulating expression of the gene of interest to a cell, tissue, and/or organ.

Further aspects of the present disclosure provide a method of identifying one or more oligonucleotides capable of upregulating gene expression comprising:
(a) immunoprecipitating ILF3 from a eukaryotic cell; and
(b) detecting one or more ribonucleic acids bound to ILF3, there by identifying oligonucleotides capable of upregulating gene expression.

In some embodiments, the eukaryotic cell comprises a nonsense-mediated decay vector (NMD) vector encoding an mRNA of interest or a homolog thereof and the method comprises identifying fragments of the mRNA of interest or homolog thereof that are bound to ILF3. In some embodiments, the cell has been transfected with an oligonucleotide comprising a segment of a mRNA of interest or a homolog thereof. In some embodiments, the detecting comprises sequencing one or more ribonucleic acids bound to ILF3. In some embodiments, a method described herein further comprising producing an engineered nucleic acid comprising the nucleic acid sequence encoding or a portion of the nucleic acid encoding the one or more oligonucleotides capable of upregulating gene expression. In some embodiments, the engineered nucleic acid is a guide RNA. In some embodiments, the engineered nucleic acid is an antisense oligonucleotide. In some embodiments, the engineered nucleic acid is a trigger nucleic acid. In some embodiments, the engineered nucleic acid is a trigger ribonucleic acid. In some embodiments, the nucleic acid is a trigger deoxyribonucleic acid.

Further aspects of the present disclosure provide a ribonucleoprotein complex comprising a ILF3 sequence and a ribonucleic acid that is less than 300 nucleotides in length. In some embodiments, the ribonucleic acid is less than 32 nucleotides in length. In some embodiments, the ribonucleic acid comprises a sequence that is at least 90% identical to any one of SEQ ID NOs: 37-40 and 87-88. In some embodiments, the ILF3 sequence comprises an amino acid sequence that is at least 90% identical to one or more of SEQ ID NOs: 1-14.

Further aspects of the present disclosure provide a ribonucleoprotein complex comprising a ILF3 sequence and a trigger ribonucleic acid. In some embodiments, the trigger ribonucleic acid comprises:

(SEQ 99)
(a) CAUCCCU;
(SEQ 100)
(b) AUCCCUG;
(SEQ 101)
(c) CCCAUCC;
(SEQ 102)
(d) CACUUCC;
(SEQ 103)
(e) UCCCUUC;
(SEQ 104)
(f) UCCCAUC;
(SEQ 105)
(g) UCCCCUC;
(SEQ 106)
(h) CCCUUCU;
(SEQ 107)
(i) CCCUCUU;
and/or
(SEQ 108)
(j) CCUACCC.

In some embodiments, the trigger nucleic acid comprises ATG or AUG at the 5′ end. In some embodiments, the trigger nucleic acid comprises TAA or UAA at the 3′ end. In some embodiments, the trigger nucleic acid comprises a 5′ cap. In some embodiments, the 5′ cap is selected from the group consisting of 3′-O-Me-m⁷G(5′)ppp(5′)G; m⁷G(5′)ppp(5′)G; G(5′)ppp(5′)G; m⁷G(5′)ppp(5′)A; and G(5′)ppp(5′)A.

Further aspects of the present disclosure provide an engineered nucleic acid that targets an antisense transcript, wherein the engineered nucleic acid is at least 90% identical to any one of SEQ ID NOs: 37-40, 49-55, 58-60, and 87-88.

Further aspects of the present disclosure provide an engineered nucleic acid that targets an antisense transcript, wherein the ribonucleic acid is the one or more oligonucleotides that are capable of upregulating expression of a gene of interest identified by any of the methods described herein.

Further aspects of the present disclosure provide a composition comprising a lipid nanoparticle that encapsulates any of the ribonucleoprotein complexes or engineered nucleic acids described herein.

Further aspects of the present disclosure provide a lipid nanoparticle that encapsulates any of the ribonucleoprotein complexes or engineered nucleic acids described herein.

Further aspects of the present disclosure provide a host cell comprising any of the non-naturally occurring proteins, fusion proteins, engineered nucleic acids, viruses, ribonucleoprotein complexes, lipid nanoparticles, or compositions described herein. In some embodiments, the host cell is a eukaryotic host cell. In some embodiments, the host cell is a mouse cell. In some embodiments, the host cell is a human cell.

Further aspects of the present disclosure provide a kit comprising any of the non-naturally occurring proteins, fusion proteins, engineered nucleic acids, viruses, ribonucleoprotein complexes, lipid nanoparticles, or compositions described herein.

Further aspects of the present disclosure provide a method of increasing expression of a gene of interest comprising administering to a cell, tissue, or organ any of the non-naturally occurring proteins, fusion proteins, engineered nucleic acids, viruses, ribonucleoprotein complexes, lipid nanoparticles, or compositions described herein.

Further aspects of the present disclosure provide a method of increasing expression of a gene of interest in a subject comprising administering to the subject any of the non-naturally occurring proteins, fusion proteins, engineered nucleic acids, viruses, ribonucleoprotein complexes, lipid nanoparticles, or compositions described herein. In some embodiments, the gene of interest is ACTG1, ACTG2, CDK9, REL, BDNF, or SOX9.

Further aspects of the present disclosure provide a method of treating a disease characterized by a decrease in expression of a gene of interest comprising administering to the subject any of the non-naturally occurring proteins, fusion proteins, engineered nucleic acids, viruses, ribonucleoprotein complexes, lipid nanoparticle, or compositions described herein.

Further aspects of the present disclosure provide a method of treating a disease characterized by a decrease in expression of a gene of interest comprising administering to a subject a trigger nucleic acid to increase expression of the gene of interest, optionally wherein the trigger nucleic acid is an antisense oligonucleotide or a trigger ribonucleic acid and/or the trigger nucleic acid comprises a sequence that is at least 90% identical to any one of SEQ ID NO: 37-40, 49-55, 58-60, and 87-88.

Further aspects of the present disclosure provide a method of increasing expression of a gene in a cell comprising administering a trigger nucleic acid to increase expression of the gene of interest, optionally wherein the trigger nucleic acid is an antisense oligonucleotide or a trigger ribonucleic acid and/or the trigger nucleic acid comprises a sequence that is at least 90% identical to any one of SEQ ID NOs: 37-40, 49-55, 58-60, and 87-88. In some embodiments, the trigger nucleic acid comprises ATG or AUG at the 5′ end. In some embodiments, the trigger nucleic acid comprises TAA or UAA at the 3′ end. In some embodiments, the trigger nucleic acid comprises a 5′ cap. In some embodiments, the 5′ cap is selected from the group consisting of 3′-O-Me-m⁷G(5′)ppp(5′)G; m⁷G(5′)ppp(5′)G; G(5′)ppp(5′)G; m⁷G(5′)ppp(5′)A; and G(5′)ppp(5′)A. In some embodiments, the trigger nucleic acid is encapsulated in a lipid nanoparticle.

In some embodiments, the engineered nucleic acid comprises one of the one or more oligonucleotides that are capable of upregulating expression of a gene of interest identified by any of the methods described herein. In some embodiments, the engineered nucleic acid comprises a fragment of one of the one or more oligonucleotides that are capable of upregulating expression of a gene of interest identified by any of the methods described herein. In some embodiments, the engineered nucleic acid is 16 to 30 nucleotides in length. In some embodiments, the engineered nucleic acid is less than 300 nucleotides in length.

Further aspects of the present disclosure provide a method of treating a disease characterized by a decrease in expression of a gene of interest comprising deactivating one or more antisense transcripts of the gene of interest to increase expression of the gene of interest in a subject.

Further aspects of the present disclosure provide a use of any of the non-naturally occurring proteins, fusion proteins, the engineered nucleic acids, the viruses, the ribonucleoprotein complexes, the lipid nanoparticles, or the compositions provided herein to treat a subject with a disease. In some embodiments, the disease is an autosomal recessive disease. In some embodiments, the disease is a haploinsufficiency disease. In some embodiments, the disease is a cancer.

Further aspects of the present disclosure provide a method comprising inducing RNA decay of the mRNA of a first gene in a cell to increase expression of a second gene in a cell, wherein the first gene is a perturbed gene set forth in Table 7 and the second gene is a corresponding adapting gene set forth in Table 7.

Further aspects of the present disclosure provide a method comprising inducing RNA decay of the mRNA of ACTG1 to increase expression of a second gene in a cell, wherein the second gene is a corresponding adapting gene set forth in Table 8.

The details of certain embodiments of the invention are set forth in the Detailed Description of Certain Embodiments, as described below. Other features, objects, and advantages of the invention will be apparent from the Definitions, Examples, Figures, and Claims. It should be understood that the aspects described herein are not limited to specific embodiments, methods, or configurations, and as such can, of course, vary. It is also to be understood that the terminology used herein is for the purpose of describing particular aspects only and, unless specifically defined herein, is not intended to be limiting.

### BRIEF DESCRIPTION OF THE DRAWINGS

The accompanying drawings, which constitute a part of this specification, illustrate several embodiments of the invention and together with the description, provide non-limiting examples of the invention.

FIGS. 1A-1D include data showing NF110 deletion domain rescues in ACTG1 knockout (KO); ILF3 knockout (KO) cells. FIG. 1A shows a schematic of the two different ILF3 isoforms (NF90 and NF110) with the domains, wherein dsRBD is double stranded RNA binding domain, RGG is a single stranded RNA binding domain, and GQSY is a domain involved in protein-protein (ptn-ptn) interaction. FIG. 1B shows qPCR analysis showing rescue of ACTG2 upregulation upon transducing ACTG1 KO; Ilf3 KO cells with full length NF90 or NF110. FIG. 1C shows qPCR analysis results similar to FIG. 1B but rescuing with NF110 where certain domains were deleted. FIG. 1D shows qPCR results of the impact of expressing NF110 without a NLS domain in ACTG1 KO; ILF3 KO.

FIGS. 2A-2D show that NMD transgenes induce a transcriptional activation (TA) effect. FIG. 2A shows a diagram explaining the NMD transgene system. FIG. 2B shows quantitative polymerase chain reaction (qPCR) analysis of mRNA of ACTG2 and endogenous ACTG1 (detected by a primer binding to the 5′UTR which is missing in the NMD transgene) expression levels upon inducing the ACTG1 NMD transgene cells with doxycycline relative to control GFP-2A-RFP NMD vector. The data show that the transgene upregulated ACTG1 and ACTG2 in an ILF3 dependent manner. FIGS. 2C and 2D show qPCR analysis of endogenous SOX9 (FIG. 2C) or BDNF (FIG. 2D) expression upon inducing the SOX9 (FIG. 2C) or BDNF (FIG. 2D) NMD transgene MEFs (FIG. 2C) or HEK293 Ts (FIG. 2D) with doxycycline. n=3 biologically independent samples. Control expression levels were set at 1. Data are mean±s.d., and a two-tailed Student's t-test was used to calculate P values.

FIGS. 3A-3F show that trigger screens identified a 75-nt region in ACTG1 that was sufficient to promote ACTG2 expression levels. FIG. 3A shows a trigger screen design for the ACTG1 NMD system used to identify regions responsible for ACTG2 upregulation using FLOW-FISH. FIG. 3B shows a volcano plot of log 2 fold change of enrichment of different triggers enrichment in the top 10% (ACTG2: Rpl13a expressing cells) over the bottom 10% and P-values from the trigger screen, with each point representing one trigger. Points marked as B show statistically significant triggers. Points marked as C represent control triggers where none scored as significant. FIG. 3C shows a diagram showing the map of the significant triggers to ACTG1 mRNA sequence, where it appears that most significant triggers share a 75-nucleotide region. SEQ ID NO: 82 is shown. FIG. 3D shows a diagram showing the map of the significant triggers to ACTG2 mRNA sequence and shows that the significant triggers shared extensive homology with ACTG2. Light grey spaces in the arrows at the top of the figure represent mismatches. FIG. 3E shows alignment of the identified 75-nucleotide region from the trigger screen to ACTG2. To validate the trigger screen results, the 75-nucleotide region was cloned into the NMD2 ptrex vector. FIG. 3F shows qPCR analysis of endogenous ACTG1 and ACTG2 expression upon inducing the 75-nt trigger NMD transgene with doxycycline relative to the control GFP-21-RFP NMD vector.

FIG. 4 shows representative screenshot of reads obtained from the small RNA sequencing upon ILF3 native RIP and mapped to the 75-nucleotide region of ACTG1. Each arrow is a sequenced RNA with grey arrows being unique matches and white sequences being multimappers. RNAs for the RNA transfection assays were chosen from the RNAs appearing in the sequencing data.

FIGS. 5A-5D show that trigger RNA transfections induced a TA response. FIGS. 5A and 5B show qPCR analysis of ACTG2 expression levels upon transfecting the indicated RNA relative to control. The data show that only RNAs of sizes 24, 27, and 31 led to a significant, but mild upregulation of ACTG2. FIG. 5C shows qPCR analysis of the indicated genes upon transfecting a combination of the 24, 27, and 31 RNAs that showed mild upregulation when transfected alone. The control used in FIG. 5C is the same as that of FIG. 5A. FIG. 5D shows qPCR analysis of ACTG2 expression levels upon transfecting the combination of the three different RNAs but with mismatches.

FIG. 6 shows that ASOs targeting antisense RNA in ACTG2 trigger screen identified regions that induced upregulation of the sense ACTG2. The dots, as indicated by the letter A, represent controls and the dots, as indicated by the letter B, represent the non-control experiments. n=3 biologically independent samples. Control expression levels were set at 1. Data are mean±s.d., and a two-tailed Student's t-test was used to calculate P values.

FIG. 7 shows that targeting dCas13-NF110 to antisense RNAs promoted gene expression. Quantitative polymerase chain reaction (qPCR) analysis of ACTG2, CDK9 and REL expression levels following transduction of the indicated cell line (grey bars: wt MEFs expressing dCas13-2A-GFP control cell line, and white bars: wt MEFs expressing dCas-NF110 cell line) with the indicated gRNAs targeting the indicated genes is shown. The dots, as indicated by the letter A, represent control non-targeting gRNA and the dots, as indicated by the letter B, represent experimental targeting gRNAs. Abbreviations are as follows: AS: antisense RNA; ex: exon; intr: intron. n=3 biologically independent samples. Control expression levels were set at 1. Data are mean±s.d., and a two-tailed Student's t-test was used to calculate P values.

FIGS. 8A-8D show more examples of gRNAs targeting antisense RNAs of ACTG2, CDK9, REL and SOX9 in cells expressing dCas13-NF110. qPCR analysis of ACTG2, CDK9, REL and SOX9 expression levels following transduction of wt MEFs expressing dCas-NF110 cell line with the indicated gRNAs targeting antisense RNAs in the indicated genes is shown. Black dots represent control non-targeting gRNA and grey dots represent experimental targeting gRNAs. Abbreviations are as follows: AS: antisense RNA; ex: exon; intr: intron. n=3 biologically independent samples. Control expression levels were set at 1. Data are mean±s.d., and a two-tailed Student's t-test was used to calculate P values. For some of the graphs, the control black dots are the same as those in FIG. 7 as they were part of the same experiment. The magnitude of upregulation varied depending on the gRNA, suggesting that optimization of the gRNA design and the targeted antisense RNA region influence the outcome.

FIG. 9 shows that targeting dCas13-NF110 to ACTG2 sense RNA promoted gene expression. Quantitative polymerase chain reaction (qPCR) analysis of ACTG2 expression levels following transduction of the indicated cell line (grey bars: wt MEFs expressing dCas13-2A-GFP control cell line, and white bars: wt MEFs expressing dCas-NF110 cell line) with the indicated gRNAs targeting the indicated genes is shown. The dots, as indicated by the letter A, represent control non-targeting gRNA and the dots, as indicated by the letter B, represent experimental targeting gRNAs. Abbreviations are as follows: AS: antisense RNA; ex: exon; intr: intron. n=3 biologically independent samples. Control expression levels were set at 1. Data are mean±s.d., and a two-tailed Student's t-test was used to calculate P values.

FIG. 10 shows more examples of targeting dCas13-NF110 to ACTG2 sense RNA promoted gene expression. qPCR analysis of ACTG2 expression levels following transduction of wt MEFs expressing dCas-NF110 cell line with the indicated gRNAs targeting ACTG2 sense RNA is shown. The dots, as indicated by the letter A, represent control non-targeting gRNA and the dots, as indicated by the letter B, represent experimental targeting gRNAs. Abbreviations are as follows: ex: exon; intr: intron. n=3 biologically independent samples. Control expression levels were set at 1. Data are mean±s.d., and a two-tailed Student's t-test was used to calculate P values. The control black dots are the same as those in FIG. 9 as they were part of the same experiment. Stronger magnitude of upregulation upon targeting sense RNAs compared to antisense was observed.

FIG. 11 shows that targeting dCas13-NF110 to antisense RNAs in regions in ACTG2 identified from a trigger screen led to stronger upregulations than those obtained by random designs. qPCR analysis of ACTG2 expression levels following transduction of the indicated cell line (grey bars: wt MEFs expressing dCas13-2A-GFP control cell line, and white bars: wt MEFs expressing dCas-NF110 cell line) with the indicated gRNAs targeting antisense RNAs in ACTG2 as identified by a trigger screen is shown. The dots, as indicated by the letter A, represent control non-targeting gRNA and the dots, as indicated by the letter B, represent experimental targeting gRNAs. Abbreviations are as follows: AS: antisense RNA; ex: exon. n=3 biologically independent samples. Control expression levels were set at 1. Data are mean±s.d., and a two-tailed Student's t-test was used to calculate P values.

FIGS. 12A-12C include data comparing trigger nucleic acids of different lengths on ACTG2 expression. FIG. 12A shows the location of the significant triggers identified from the trigger screen mapped onto ACTG2 mRNA sequence. Light grey gaps in the arrows at the top of the figure indicate a mismatch. Sequences shown below the ACTG2 exons indicate that the identified ACTG1 75 nucleotide trigger shares extensive homology with a 75-nucleotide region in ACTG2, in addition to the 24, 27 and 31 nucleotide trigger RNAs used in FIGS. 5A-5C. Bolded letters indicate a mismatch. The following sequences are shown (top to bottom): SEQ ID NO: 83, SEQ ID NO: 82, SEQ ID NO: 84, SEQ ID NO: 85, and SEQ ID NO: 86. FIG. 12B shows qPCR analysis of ACTG2 mRNA expression levels in wild-type (WT) MEFs expressing the trigger-screen-identified 75-nucleotide trigger region, or Actg1 without the 75-nucleotide trigger region in the NMD vector relative to control GFP-2A-RFP. FIG. 12C shows qPCR analysis of ACTG2 mRNA expression levels in WT (white bars) or ILF3 knockdown (grey bars) MEFs transfected with the indicated trigger RNAs. The 24, 27, 31 nt RNAs were selected as they individually induced a significant mild upregulation of ACTG2 when transfected to cells (FIGS. 5A-5B).

FIGS. 13A-13G shows comparative CRISPRn and CRISPRi Perturb-seq experiments reveal widespread TA responses in human K562 cells and confirm a global requirement for ILF3 in TA. FIG. 13A shows the logic behind the Perturb-seq experiments comparing CRISPRn and CRISPRi. Abbreviations are as follows: ORF: open reading frame. PTC: premature termination codon. In the graph, different sets of gene pairs are indicated as follows: A: all gene pairs (n=683,012), B: all gene pairs exhibiting some sequence similarity (n=116,403). Thin lines represent subsets of the thick B line: C: all gene pairs where sequence similarity lies in the gene body of the observed (assessed) gene (n=40,973); D: all gene pairs where sequence similarity lies in the promoter of the observed (assessed) gene (n=40,971); E: all gene pairs where sequence similarity lies in a putative enhancer of the observed (assessed) gene (n=85,817). P values are for each dataset in relation to All gene pairs, and were calculated by Mann-Whitney U test. FIG. 13B shows the cumulative distribution of the ratio of expression fold change upon perturbing a gene with CRISPRn relative to that upon CRISPRi perturbation. FIG. 13C shows qPCR analysis of ELP3 and ZEB1 mRNA expression levels in K562-Cas9 cells that express a non-targeting (control), or CSNK1E sgRNA, or CSNK1E and UPF1 gRNAs. FIG. 13D shows qPCR analysis of DDX59, MAN2A2 and RGS16 mRNA expression levels in K562-Cas9 cells that express a non-targeting (control), or DDX21 sgRNA, or DDX21 and UPF1 gRNAs. FIG. 13E shows qPCR analysis of ELP3 and ZEB1 mRNA expression levels in K562-Cas9 cells that co-express either a non-targeting (control), or CSNK1E sgRNA, along with ILF3 sgRNAs. FIG. 13F shows qPCR analysis of DDX59, MAN2A2 and RGS16 mRNA expression levels in K562-Cas9 cells that co-express either a non-targeting (control), or DDX21 sgRNA, along with ILF3 sgRNAs. (C-F) n=3 biologically independent samples. Wild-type or control expression levels were set at 1 for each assay. Data are mean±s.d., and a two-tailed Student's t-test was used to calculate P values. FIG. 13G shows the fold change of expression levels of the observed adapting genes in TA-candidate gene pairs in CRISPRn Perturb-seq experiment performed on WT and ILF3 KO K562 cells. Red lines indicate a decrease in fold change by more than 1.5, grey lines indicate changes that are a decrease or an increase of less than 1.5, and blue line indicates an increase in fold change by more than 1.5. n=636 gene pairs. Genes that were identified to be differentially expressed upon ILF3 knockdown in WT cells (see, e.g., Replogle et al., Cell. 2022 Jul. 7; 185(14):2559-2575.e28.) were removed from the analysis to avoid analyzing genes that are regulated by ILF3 independent of TA. Mann-Whitney U test was used to calculate P value.

FIGS. 14A-14H shows CRISPRn and CRISPRi Perturb-seq experiments lead to successful perturbations, and uncovers candidate TA responses. FIG. 14A shows a heatmap of the expression levels of the genes targeted in the CRISPRn perturb-seq experiment across each perturbation relative to non-targeting control gRNAs. The darker diagonal line indicates efficient NMD of each gene upon its perturbation. Fold change >1 was kept at 1 to allow for visualization of NMD. White spots indicate that the analyzed gene was not expressed in K562s. FIG. 14B Shows expression levels of the observed (assessed) genes within the identified TA-candidate and control pairs upon CRISPRn or CRISPRi-mediated perturbation, relative to control cells (top), and expression levels of the observed (assessed) genes within the identified TA-candidate and control pairs upon CRISPRn or CRISPRi-mediated perturbation, relative to control cells except that expression levels were changed to 1 if the change observed was not significant after P value correction. Each dot represents a gene pair (bottom). FIG. 14C shows the number of significant differentially expressed genes (DEGs) upon perturbation of a gene by CRISPRn versus CRISPRi. Each dot represents a perturbed gene. Both CRISPRn and CRISPRi perturbations of a given gene lead to a close number of DEGs. FIG. 14D shows the embedding of all perturbed genes for each method of perturbation, obtained by applying UMAP to the corrected P-values of the transcriptomic response upon perturbation. For each perturbed gene, CRISRPn and CRISPRi transcriptomes cluster together, indicating similarly successful perturbation of the genes using either methods, and no major batch effects or other confounders influencing the results. FIG. 14E shows the euclidean distance in high dimensional space between transcriptomes upon CRISPRn or CRISPRi perturbation of a given gene (dot in this plot) plotted against expression levels of the perturbed gene relative to control cells when perturbing it with CRISPRi (top) or CRISPRn (bottom). FIG. 14F shows the euclidean distance in high dimensional space between transcriptomes upon CRISPRn or CRISPRi perturbation of a given gene (dot in this plot) plotted against the number of identified control (top) and TA-candidate (bottom) observed genes for each perturbed gene. Data for FIGS. 14E and 14F show that the differences in transcriptome profiles following CRISPRn versus CRISPRi perturbations was independent of the efficiency of NMD or knockdown, respectively, but likely due to TA-responses. FIG. 14G shows the number of TA-candidate gene pairs identified for each perturbed gene (dot) plotted against expression levels of the perturbed gene relative to control cells when perturbing it with CRISPRi. Essential genes were identified from the Cancer Dependency Map common essential genes as defined in the year 2020, Quarter 1. Data shows that the number of TA-candidate gene pairs was independent of gene essentiality or knockdown levels with CRISPRi-mediated perturbation. FIG. 14H shows the expression levels (log 2[TPM+1]) of the perturbed genes in WT K562s as identified from the Epimap dataset, plotted against the number of identified control gene pairs (left) and TA-candidate (right). Data shows that the number of TA-candidate or control gene pairs was independent of the expression levels of the perturbed gene in WT cells.

FIG. 15A-15F show the features of TA candidate gene pairs. FIG. 15A shows the cumulative distribution of negative log₁₀ E-values from the best BLAST alignment per gene pair, for TA-candidate gene pairs (as indicated by the letter B) or Control gene pairs (as indicated by the letter A). Gene pairs were included only if they have at least one BLAST alignment with negative log 10 E-values >0. n=101 TA-candidate pairs, 341 Control pairs. A higher −log₁₀ E-value indicates higher level of similarity. FIG. 15B shows the distance between PCA components of the gene pairs based of association patterns to health traits from a UK biobank phenotypes exome-wide association results from Backman et al., Nature. 2021 November; 599(7886):628-634 across different number of components. For each gene pair in a set, lower distance is indicative that the two genes have more similar association patterns. n=681 and 2315, respectively. FIG. 15C shows the cumulative distribution of the alignment length divided by the length of the aligned-to feature (i.e., gene body, promoter, or enhancer) of TA-candidate (as indicated by the letter A) or Control (as indicated by the letter B) gene pairs. These data show that TA-candidate gene pairs had longer alignment lengths than the Control pairs. Gene pairs were included only if they have at least one BLAST alignment with negative log 10 E-values >0. n of TA-candidate alignments:Control alignments: 46:82 (enhancer_ABC), 31:110 (enhancer_epimap), 215:317 (enhancer_eRNA-lidschreiber), 71:100 (enhancer_eRNA-Yulab), 161:416 (promoter_2500 bp) 2842:8659 (genebody). FIG. 15D shows the cumulative distribution of negative log₁₀ E-values from the best BLAST alignment per gene pair, for TA-candidate gene pairs (as indicated by the letter B) or Control gene pairs (as indicated by the letter A) where the alignment to the observed gene lies in a putative enhancer region. Gene pairs were included only if they have at least one BLAST alignment with negative log₁₀ E-values >0. A higher −log₁₀ E-value indicates higher level of similarity. n of TA-candidate alignments:Control alignments: 22:44 (enhancer_ABC), 15:62 (enhancer_epimap), 31/:5 (enhancer_eRNA-lidschreiber), 24:56 (enhancer_eRNA-Yulab). FIGS. 15C and 15D show that for gene pairs where similarity lies in a putative enhancer region of the observed gene, alignments to enhancers where evidence of enhancer RNA (eRNA) transcription was present displayed higher levels of similarity and longer lengths in TA-candidate pairs relative to Control pairs, which was not always the case for enhancers without direct evidence of eRNAs. These data indicates that eRNAs could be a target for mRNA decay intermediates. FIG. 15E shows that in WT cells, the observed (adapting) genes found only within TA-candidate gene pairs (n=349) have significantly lower levels of H3K4me3 at their promoters than observed genes not in any TA-candidate pairs (n=7521), indicating that those genes would be more poised to increase in expression through COMPASS-complex mediated H3K4me3 deposition, in accordance with how TA is induced (see, e.g., El-Brolosy et al. Nature. April; 568(7751):193-197 and Ma et al. Nature. April; 568(7751):259-263). H3K4me3 data in WT K562s were obtained from Boix et al. Nature. 2021 May; 593(7858):238-243. FIG. 15F shows the cumulative distribution of the position of the alignment start position relative to the gene body length for TA-candidate (as indicated by the letter B) or Control (as indicated by the letter A) gene pairs. These data show that position of the alignment between the perturbed gene's mRNA relative to the adapting gene's body does not have a strong influence TA (i.e., the alignment lying in the 5′ vs 3′ of the adapting gene has limited influence). n of alignments=2842 alignments (TA-candidate) and 8659 (Control). For FIGS. 14A-14F P values were calculated by Mann-Whitney U test.

FIGS. 16A-16G show ILF3 is a global mediator of TA. FIG. 16A shows a representative western blot analysis of ILF3 in K562 cells expressing non-targeting (control), or ILF3 sgRNAs. FIG. 16B shows the log 2 of the ratio of fold change of gene expression relative to control upon perturbing a gene with CRISPRn in WT to that of ILF3 knockout K562s. Plotted gene pairs are TA-candidate gene pairs (as indicated by the letter A), all other non-TA-candidate gene pairs where the observed (assessed) gene is significantly upregulated (Fold Change >1, Padj<0.05) in the CRISPRn Perturb-seq dataset from WT cells (as indicated by the letter B), and a further subset of that containing only gene pairs where the observed (assessed) gene was upregulated with a Fold Change >1.5 and Padj<0.05 both upon CRISPRn or CRISPRi perturbation in WT cells. Genes that were identified to be differentially expressed upon ILF3 knockdown in WT cells (see, e.g., Replogle et al., Cell. 2022 Jul. 7; 185(14):2559-2575.e28.) were removed from the analysis to avoid analyzing genes that are regulated by ILF3 independent of TA. n=636, 1546 and 240, respectively. FIG. 16C shows the log 2 of the ratio of fold change of gene expression relative to control upon perturbing a gene with CRISPRn in WT to that of ILF3 knockout K562s. Plotted gene pairs are non-TA-candidate pairs where the observed (assessed) genes were found in the TA-candidate list. Plot shows that the median of the log 2 change for those observed genes in non-TA-candidate pairs is 0, supporting that those genes are not directly regulated by ILF3. FIG. 16D shows a heatmap showing the expression levels of the genes targeted in the CRISPRn perturb-seq experiment, performed in ILF3 knockout K562s, across each perturbation relative to non-targeting control gRNAs. The darker diagonal line indicates efficient NMD of each gene upon its perturbation. Fold change >1 was kept at 1 to allow for visualization of NMD. White spots indicate that the analyzed gene was not expressed in K562s. FIG. 16E shows the cumulative distribution of E-values obtained upon BLASTing ILF3 motifs (identified from eCLIP-seq ENCODE data (see e.g., Van Nostrand et al. Nature. 2020 July; 583(7818):711-719 and Feng et al. Mol Cell. 2019 Jun. 20; 74(6):1189-1204.e6)) with the alignment region from the perturbed-gene to the assessed (observed) gene within TA-candidate (as indicated by the letter B) or Control (as indicated by the letter A) gene pairs. Only gene pairs found to have at least one high-confidence match (motif matches with a P value <0.0001) to an ILF3 motif were included for each pair type. N=131 (TA-candidate), and 231 (Control). FIG. 16F shows the percentage of gene pairs where the BLAST-identified alignment region from the perturbed-gene to the assessed (observed) gene had a high-confidence match (motif matches with a P value <0.0001) to an ILF3 motif identified from eCLIP-seq ENCODE data. TA-candidate: 131/475 and Control: 231/1660. FIG. 16G shows the cumulative distribution of the observed (adapting) genes' expression fold change in the CRISPRn perturb-seq dataset. The two lines are TA-candidate pairs where the perturbed gene's alignment region to the observed (adapting) gene has a high-confidence match to an ILF3 motif (orange line, n=131) versus no match to any ILF3 motif (teal line, n=600). For FIGS. 16B, 16E, and 16G P values were calculated by Mann-Whitney U test.

FIGS. 17A-17C shows ILF3 is recruited to adapting genes' loci, and its recruitment is sufficient to promote gene expression. FIG. 17A shows IGV tracks of the Actg2 locus showing PRO-seq signals in WT cells from the positive and negative strands. Abbreviations are as follows: A.S.: antisense. FIG. 17B shows log 2 fold change of genes' IP/input signal in ILF3 nuclear RIP-seq experiments in Actg1-NSD cells relative to that in WT MEFs plotted against −log₁₀ P values as calculated by DeSeq2, showing genes where Log₂ Fold change was >0. Actg1 and Actg2 are both highlighted. FIG. 17C shows RNA-seq (see, e.g., El-Brolosy et al. Nature. April; 568(7751):193-197) and PRO-seq analyses showing the Log₂ fold change of gene expression between the indicated cell lines for genes identified from RIP-seq analysis to be more associated with ILF3 in Actg1-NSD cells relative to WT (identified as L2F >1 and Pva1 <0.01). Fewer genes appear in the PRO-seq graphs relative to RNA-seq as fewer genes meet the number of reads cutoff to be considered detected in the PRO-seq experiment.

FIG. 18 shows gene-level enrichment of the average scores of the sgRNAs in the bottom 30% of Actg2/Rpl13a expressing cells relative to the top 30% of expressing cells, plotted against MAGeCK-calculated P values obtained from two independent replicates of a genome-wide CRISPR screen in Actg1-NSD cells. Highlighted genes represent a validation of the efficiency of the screen: i) RNA decay factors and the COMPASS complex previously shown to influence TA also identify as hits in the screen ii) gRNAs targeting Actg2 but not Actg1 decrease Actg2 FISH signal confirming the specificity of the used FISH probes. gRNAs targeting Actb also lead to increased Actg2 signal.

FIGS. 19A and 19B show ILF3-dependent recruitment of epigenetic modifiers in transcriptional adaptation. FIG. 19A shows gene-level enrichment of the average scores of the sgRNAs in the bottom 30% of Actg2/Rpl13a expressing cells relative to the top 30% of expressing cells, plotted against MAGeCK-calculated P values obtained from two independent replicates of a genome-wide CRISPR screen in Actg1-NSD cells. Genes scoring as hits in the counter screen in WT cells were removed from the analysis. Highlighted is Smarca4 (BRG1) a core component of the SWI/SNF chromatin remodeling complex. FIG. 19B shows ChIP-qPCR analysis of BRG1, PRMT1 and YY1 at the Actg2 locus in Actg1-NSD and Actg1-NSD;ΔIlf3 cells compared to WT MEFs. Data points here reflect data using different primer pairs. Control expression levels were set at 1 for each assay. Data are mean±s.d., and a two-tailed Student's t-test was used to calculate P values.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

“AAV” or “adeno-associated virus” is a nonenveloped virus that is capable of carrying and delivering nucleic acids (e.g., engineered nucleic acids) and belongs to the genus Dependoparvovirus. In some instances, an AAV is capable of delivering a nucleic acid encoding an RNA-binding protein and/or recombinant nucleic acid described herein. In general, AAV does not integrate into the genome. The tissue-specific targeting capabilities of AAV is often determined by the AAV capsid serotype. Non-limiting serotypes of AAV include AAV1, AAV2, AAV3, AAV4, AAV5, AAV6, AAV7, AAV8, AAV9, AAV10, AAV11, AAV PHP.b, and variants thereof.

The term “administer,” “administering,” or “administration” refers to implanting, absorbing, ingesting, injecting, inhaling, or otherwise introducing a protein and/or nucleic acid described herein, or a composition thereof, in or on a subject.

The term “cancer” refers to a class of diseases characterized by the development of abnormal cells that proliferate uncontrollably and have the ability to infiltrate and destroy normal body tissues. See e.g., Stedman's Medical Dictionary, 25th ed.; Hensyl ed.; Williams & Wilkins: Philadelphia, 1990. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstrom's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

The term “Cas13” or “Cas13 protein” refers to a class 2 type VI RNA-guided RNA-targeting protein. Naturally occurring Cas13 proteins are RNA endonucleases with two 0 (higher eukaryotes and prokaryotes nucleotide-binding) domains for RNA cleavage. Naturally occurring Cas13 proteins use the Helical-1, Lid, and Helical-2 domains to recognize the crRNA. In naturally-occurring CRISPR systems comprising Cas13, Cas13 assembles with crRNA to recognize target RNAs and upon binding to a target RNA, Cas13 undergoes a conformation change that activates the nuclease domain of the Cas13 protein to cleave the target RNA. In some embodiments, a Cas13 protein is a Cas13a, Cas13b, Cas13C, Cas13d (CasRx), or Cas13bt protein. See also, e.g., Cox et al., Science. 2017 Nov. 24; 358(6366): 1019-1027. In some embodiments, the Cas13 proteins for use in this disclosure do not comprise nuclease activity and therefore do not cleave RNA target sequences. For example, a Cas13 protein for use herein may lack one or more HEPN domains and/or comprise one or more mutations in a HEPN domain that inactivates the nuclease activity of the Cas13 protein. In some embodiments, a Cas13 protein is a CasRx protein comprising the following mutations relative to wild-type CasRx: R239A/H244A/R858A/H863A. In some embodiments, a Cas13 protein comprises the following domains: Helical-1, Lid, and Helical-2.

A sequence “complementary” to a portion of an RNA, refers to a sequence having sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex. A nucleic acid may be “self-complementary” and comprise regions that are complementary to one another that hybridize to form a secondary structure. For example, a single-stranded nucleic acid may comprise self-complementary regions that hybridize and form a secondary structure.

The terms “condition,” “disease,” and “disorder” are used interchangeably. In some embodiments, a diseased cell, tissue, organ, or subject with a disease is characterized by a decrease in expression of a gene of interest as compared to a cell, tissue, organ, or subject without the disease. In some embodiments, a disease characterized by a decrease in expression of a gene of interest is a haploinsufficiency disorder. Haploinsufficiency is a dominant phenotype in diploid organisms in which a single functional copy of a gene is insufficient to maintain normal function. Non-limiting examples of haploinsufficiency disorders include familial hypercholesteremia, autosomal dominant polycystic kidney disease (APKD), neurofibromatosis, and hypertrophic cardiomyopathy. In some embodiments, a disease characterized by a decrease in expression of a gene of interest is an autosomal recessive disorder, in which two mutated alleles of a gene are required to produce a phenotype. In some embodiments, an autosomal recessive disorder is caused by a mutation in a gene that has a paralog, including but not limited to Duchenne muscular dystrophy (DMD), sickle cell anemia, hemochromatosis, alpha-1 antitrypsin deficiency, and beta thalassemia intermedia. For example, DMD is often caused by mutations in the dystrophin gene. Utrophin is a paralog of DMD, which can partially rescue the DMD phenotype in animal models. See, e.g., Tinsley et al., Nat. Med. 1998; 4:1441-1444. It has also been observed that expression of the fetal gene paralog γ-globin may be used to ameliorate sickle cell anemia or β-globin disease, sickle cell disease and β-thalassemia. Hemochromatosis is commonly caused by missense mutations in HFE, which has a paralog (HFE2). Alpha-1 Antitrypsin Deficiency is often caused by a missense mutation in the SERPINA1 gene, which has several paralogs including SERPINA4. In some embodiments, a disease characterized by a decrease in expression of a gene of interest is a cancer.

The term “CRISPR” refers to a family of DNA sequences (i.e., CRISPR clusters) in bacteria and archaea that represent snippets of prior infections by a virus that have invaded the prokaryote. The snippets of DNA are used by the prokaryotic cell to detect and destroy DNA and/or RNA from subsequent attacks by similar viruses and effectively compose, along with an array of CRISPR-associated proteins and CRISPR-associated RNA, a prokaryotic immune defense system

In general, a “CRISPR system” refers collectively to transcripts and other elements involved in the expression of or directing the activity of CRISPR-associated (“Cas”) genes, including sequences encoding a Cas protein, tracr (trans-activating CRISPR) RNA (tracrRNA) sequences, and guide sequences. A guide sequence comprises at least a nucleic acid sequence that is complementary to a target sequence of interest. In some embodiments, the nucleic acid sequence that is complementary to a target sequence of interest is referred to as a CRISPR RNA (crRNA). A guide sequence may be a single guide RNA (sgRNA) (chimeric RNA) that comprises both a nucleic acid sequence that is complementary to a target sequence of interest and a tracr. Certain Cas proteins including Cas12a and Cas13a do not require a tracr. In some instances, a guide sequence does not comprise a tracr. See, e.g., Murugan et al., Mol Cell. 2017 Oct. 5; 68(1):15-25.

The term “deactivate”, “deactivating”, “deactivation”, “repress”, or “inactivate,” when used in reference to an antisense transcript of a gene, refers to the downregulation of activity of the antisense transcript. As a non-limiting example, deactivation of an antisense transcript may result from preventing the antisense transcript from binding to the mRNA or promoting degradation of the transcript.

An “effective amount” of a protein and/or nucleic acid described herein refers to an amount sufficient to elicit the desired biological response. An effective amount of a protein and/or nucleic acid described herein may vary depending on such factors as the desired biological endpoint, severity of side effects, disease, or disorder, the identity, pharmacokinetics, and pharmacodynamics of the particular protein and/or nucleic acid, the condition being treated, the mode, route, and desired or required frequency of administration, the species, age and health or general condition of the subject. In certain embodiments, an effective amount is a therapeutically effective amount. In certain embodiments, an effective amount is a prophylactic treatment. In certain embodiments, an effective amount is the amount of a protein and/or nucleic acid described herein in a single dose. In certain embodiments, an effective amount is the combined amounts of a protein and/or nucleic acid described herein in multiple doses. In certain embodiments, the desired dosage is delivered three times a day, two times a day, once a day, every other day, every third day, every week, every two weeks, every three weeks, or every four weeks. In certain embodiments, the desired dosage is delivered using multiple administrations (e.g., two, three, four, five, six, seven, eight, nine, ten, eleven, twelve, thirteen, fourteen, or more administrations).

A “engineered nucleic acid molecule” is a non-naturally occurring nucleic acid molecule. In some embodiments, the engineered nucleic acid is a nucleic acid molecule that has undergone a molecular biological manipulation, e.g., genetically engineered nucleic acid molecule. Furthermore, the term “engineered DNA molecule” or “engineered ribonucleic acid” (“engineered RNA”) refers to a nucleic acid sequence which is not naturally occurring, or can be made by the artificial combination of two otherwise separated segments of nucleic acid sequence, i.e., by ligating together pieces of DNA or RNA that are not normally continuous. Engineered nucleic acids may be produced through artificial combination often accomplished by either chemical synthesis means, or by the artificial manipulation of isolated segments of nucleic acids, e.g., by genetic engineering techniques using restriction enzymes, ligases, and similar recombinant techniques as described by, for example, Sambrook et al., Molecular Cloning, second edition, Cold Spring Harbor Laboratory, Plainview, N.Y.; (1989), or Ausubel et al., Current Protocols in Molecular Biology, Current Protocols (1989), and DNA Cloning: A Practical Approach, Volumes I and II (ed. D. N. Glover) IREL Press, Oxford, (1985); each of which is incorporated herein by reference.

An “engineered virus” is a virus (e.g., lentivirus, adenovirus, retrovirus, herpes virus, human papillomavirus, alphavirus, vaccinia virus or adeno-associated virus (AAV)) that has been isolated from its natural environment (e.g., from a host cell, tissue, or a subject) or is artificially produced.

The term “fusion protein” as used herein refers to a hybrid polypeptide which comprises protein domains from at least two different proteins. One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. A protein may comprise different domains, for example, one or more domains of an interleukin enhancer-binding factor 3 and/or one or more domains of a Cas protein. In some embodiments, a linker (e.g., a peptide linker) is present between the two proteins or two protein domains. In some embodiments, a fusion protein comprises one or more affinity tags. Non-limiting examples of affinity tags include the following tags: BP, FLAG, GST, HA, HBH, MBP, Myc, poly His, S-tag, SUMO, TAP, TRX, and V5. In some embodiments, a fusion protein comprises a nuclear localization signal sequence. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4^{th} ed., Cold Spring Harbor Laboratory Press, Cold Spring Harbor, N.Y. (2012)), the entire contents of which are incorporated herein by reference.

The term “gene” refers to a nucleic acid fragment that expresses a protein, including regulatory sequences preceding (5′-non-coding sequences) and following (3′ non-coding sequences) the coding sequence. “Native gene” refers to a gene as found in nature with its own regulatory sequences. “Chimeric gene” or “chimeric construct” refers to any gene or a construct, not a native gene, comprising regulatory and coding sequences that are not found together in nature. Accordingly, a chimeric gene or chimeric construct may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene not normally found in the host organism, but which is introduced into the host organism by gene transfer. Foreign genes can comprise native genes inserted into a non-native organism, or chimeric genes. A “transgene” is a gene that has been introduced into the genome by a transformation procedure. Exemplary genes include, but are not limited to, ACTG1, ACTG2, CDK9, REL, BDNF, or SOX9.

The term “genetic disease” refers to a disease caused by one or more abnormalities in the genome of a subject, such as a disease that is present from birth of the subject. Genetic diseases may be heritable and may be passed down from the parents' genes. A genetic disease may also be caused by mutations or changes of the DNAs and/or RNAs of the subject. In such cases, the genetic disease will be heritable if it occurs in the germline. Exemplary genetic diseases include, but are not limited to, Aarskog-Scott syndrome, Aase syndrome, achondroplasia, acrodysostosis, addiction, adreno-leukodystrophy, albinism, ablepharon-macrostomia syndrome, alagille syndrome, alkaptonuria, alpha-1 antitrypsin deficiency, Alport's syndrome, Alzheimer's disease, asthma, autoimmune polyglandular syndrome, androgen insensitivity syndrome, Angelman syndrome, ataxia, ataxia telangiectasia, atherosclerosis, attention deficit hyperactivity disorder (ADHD), autism, baldness, Batten disease, Beckwith-Wiedemann syndrome, Best disease, bipolar disorder, brachydactyl), breast cancer, Burkitt lymphoma, chronic myeloid leukemia, Charcot-Marie-Tooth disease, Crohn's disease, cleft lip, Cockayne syndrome, Coffin Lowry syndrome, colon cancer, congenital adrenal hyperplasia, Cornelia de Lange syndrome, Costello syndrome, Cowden syndrome, craniofrontonasal dysplasia, Crigler-Najjar syndrome, Creutzfeldt-Jakob disease, cystic fibrosis, deafness, depression, diabetes, diastrophic dysplasia, DiGeorge syndrome, Down's syndrome, dyslexia, Duchenne muscular dystrophy, Dubowitz syndrome, ectodermal dysplasia Ellis-van Creveld syndrome, Ehlers-Danlos, epidermolysis bullosa, epilepsy, essential tremor, familial hypercholesterolemia, familial Mediterranean fever, fragile X syndrome, Friedreich's ataxia, Gaucher disease, glaucoma, glucose galactose malabsorption, glutaricaciduria, gyrate atrophy, Goldberg Shprintzen syndrome (velocardiofacial syndrome), Gorlin syndrome, Hailey-Hailey disease, hemihypertrophy, hemochromatosis, hemophilia, hereditary motor and sensory neuropathy (HMSN), hereditary non polyposis colorectal cancer (HNPCC), Huntington's disease, immunodeficiency with hyper-IgM, juvenile onset diabetes, Klinefelter's syndrome, Kabuki syndrome, Leigh's disease, long QT syndrome, lung cancer, malignant melanoma, manic depression, Marfan syndrome, Menkes syndrome, miscarriage, mucopolysaccharide disease, multiple endocrine neoplasia, multiple sclerosis, muscular dystrophy, myotrophic lateral sclerosis, myotonic dystrophy, neurofibromatosis, Niemann-Pick disease, Noonan syndrome, obesity, ovarian cancer, pancreatic cancer, Parkinson's disease, paroxysmal nocturnal hemoglobinuria, Pendred syndrome, peroneal muscular atrophy, phenylketonuria (PKU), polycystic kidney disease, Prader-Willi syndrome, primary biliary cirrhosis, prostate cancer, REAR syndrome, Refsum disease, retinitis pigmentosa, retinoblastoma, Rett syndrome, Sanfilippo syndrome, schizophrenia, severe combined immunodeficiency, sickle cell anemia, spina bifida, spinal muscular atrophy, spinocerebellar atrophy, sudden adult death syndrome, Tangier disease, Tay-Sachs disease, thrombocytopenia absent radius syndrome, Townes-Brocks syndrome, tuberous sclerosis, Turner syndrome, Usher syndrome, von Hippel-Lindau syndrome, Waardenburg syndrome, Weaver syndrome, Werner syndrome, Williams syndrome, Wilson's disease, xeroderma piginentosum, and Zellweger syndrome.

“Homolog” or “homologous” refers to sequences (e.g., nucleic acid (e.g., engineered nucleic acid) or amino acid sequences) that share a certain percent identity (e.g., at least 5%, at least 10%, at least 15%, at least 20%, at least 25%, at least 30%, at least 35%, at least 40%, at least 45%, at least 50%, at least 55%, at least 60%, at least 65%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% percent identity). The present disclosure encompasses sequences with a certain percent identity (e.g., at least 5%, at least 10%, at least 15%, at least 20%, at least 25%, at least 30%, at least 35%, at least 40%, at least 45%, at least 50%, at least 55%, at least 60%, at least 65%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% percent identity) to any of the nucleic acid or amino acid sequences disclosed herein. Homologous sequences include but are not limited to paralogous or orthologous sequences. Paralogous sequences arise from duplication of a gene within a genome of a species, while orthologous sequences diverge after a speciation event. A functional homolog retains one or more biological activities of a wild-type protein. In certain embodiments, a functional homolog of ILF3 retains at least 10%, at least 20%, at least 30%, at least 40%, at least 50%, at least 60%, at least 70%, at least 80%, at least 90%, or at least 100% of the biological activity (e.g., transcription factor activity and/or RNA-binding activity) of a wild-type counterpart.

The term “host cell,” as used herein, refers to a cell that can host, replicate, and express a vector described herein, e.g., a vector comprising any nucleic acid molecule disclosed herein, including any nucleic acid molecule encoding a fusion protein, Cas protein, ILF3 sequence, and/or engineered nucleic acid disclosed herein.

Nucleic acid or amino acid sequence “identity,” as described herein, can be determined by comparing a nucleic acid or amino acid sequence of interest to a reference nucleic acid or amino acid sequence. The percent identity is the number of nucleotides or amino acid residues that are the same (e.g., that are identical) as between the sequence of interest and the reference sequence divided by the length of the longest sequence (e.g., the length of either the sequence of interest or the reference sequence, whichever is longer). A number of mathematical algorithms for obtaining the optimal alignment and calculating identity between two or more sequences are known and incorporated into a number of available software programs. Examples of such programs include CLUSTAL-W, T-Coffee, and ALIGN (for alignment of nucleic acid and amino acid sequences), BLAST programs (e.g., BLAST 2.1, BL2SEQ, and later versions thereof) and FASTA programs (e.g., FASTA3x, FAS™, and SSEARCH) (for sequence alignment and sequence similarity searches). Sequence alignment algorithms also are disclosed in, for example, Altschul et al., J. Molecular Biol., 215(3): 403-410 (1990), Beigert et al., Proc. Natl. Acad. Sci. USA, 106(10): 3770-3775 (2009), Durbin et al., eds., Biological Sequence Analysis: Probabilistic Models of Proteins and Nucleic Acids, Cambridge University Press, Cambridge, UK (2009), Soding, Bioinformatics, 21(7): 951-960 (2005), Altschul et al., Nucleic Acids Res., 25(17): 3389-3402 (1997), and Gusfield, Algorithms on Strings, Trees and Sequences, Cambridge University Press, Cambridge UK (1997)).

Mammalian cells of the present disclosure include human cells, primate cells (e.g., vero cells), rat cells (e.g., GH3 cells, OC23 cells) or mouse cells (e.g., MC3T3 cells). There are a variety of human cell lines, including, without limitation, human embryonic kidney (HEK) cells, HeLa cells, cancer cells from the National Cancer Institute's 60 cancer cell lines (NCI60), DU145 (prostate cancer) cells, Lncap (prostate cancer) cells, MCF-7 (breast cancer) cells, MDA-MB-438 (breast cancer) cells, PC3 (prostate cancer) cells, T47D (breast cancer) cells, THP-1 (acute myeloid leukemia) cells, U87 (glioblastoma) cells, SHSY5Y human neuroblastoma cells (cloned from a myeloma) and Saos-2 (bone cancer) cells. In some embodiments, engineered nucleic acids are delivered into human embryonic kidney (HEK) cells (e.g., HEK 293 or HEK 293T cells). In some embodiments, engineered nucleic acid vectors are delivered into stem cells (e.g., human stem cells) such as, for example, pluripotent stem cells (e.g., human pluripotent stem cells including human induced pluripotent stem cells (hiPSCs)). A stem cell refers to a cell with the ability to divide for indefinite periods in culture and to give rise to specialized cells. A pluripotent stem cell refers to a type of stem cell that is capable of differentiating into all tissues of an organism, but not alone capable of sustaining full organismal development. A human induced pluripotent stem cell refers to a somatic (e.g., mature or adult) cell that has been reprogrammed to an embryonic stem cell-like state by being forced to express genes and factors important for maintaining the defining properties of embryonic stem cells (see, e.g., Takahashi and Yamanaka, Cell 126 (4): 663-76, 2006, incorporated by reference herein). Human induced pluripotent stem cell cells express stem cell markers and are capable of generating cells characteristic of all three germ layers (ectoderm, endoderm, mesoderm).

Additional non-limiting examples of cell lines that may be used in accordance with the present disclosure include 293-T, 293-T, 3T3, 4T1, 721, 9L, A-549, A172, A20, A253, A2780, A2780ADR, A2780cis, A431, ALC, B16, B35, BCP-1, BEAS-2B, bEnd.3, BHK-21, BR 293, BxPC3, C2C12, C3H-10T1/2, C6, C6/36, Cal-27, CGR8, CHO, CML T1, CMT, COR-L23, COR-L23/5010, COR-L23/CPR, COR-L23/R23, COS-7, COV-434, CT26, D17, DH82, DU145, DuCaP, E14Tg2a, EL4, EM2, EM3, EMT6/AR1, EMT6/AR10.0, FM3, H1299, H69, HB54, HB55, HCA2, Hepa1c1c7, High Five cells, HL-60, HMEC, HT-29, HUVEC, J558L cells, Jurkat, JY cells, K562 cells, KCL22, KG1, Ku812, KYO1, LNCap, Ma-Mel 1, 2, 3 . . . 48, MC-38, MCF-10A, MCF-7, MDA-MB-231, MDA-MB-435, MDA-MB-468, MDCK II, MG63, MONO-MAC 6, MOR/0.2R, MRC5, MTD-1A, MyEnd, NALM-1, NCI-H69/CPR, NCI-H69/LX10, NCI-H69/LX20, NCI-H69/LX4, NIH-3T3, NW-145, OPCN/OPCT Peer, PNT-1A/PNT 2, PTK2, Raji, RBL cells, RenCa, RIN-5F, RMA/RMAS, S2, Saos-2 cells, Sf21, Sf9, SiHa, SKBR3, SKOV-3, T-47D, T2, T84, THP1, U373, U87, U937, VCaP, WM39, WT-49, X63, YAC-1 and YAR cells.

Some aspects of this disclosure provide cells comprising any of the constructs disclosed herein. In some embodiments, a host cell is transiently or non-transiently transfected with one or more vectors described herein. In some embodiments, a cell is transfected as it naturally occurs in a subject. In some embodiments, a cell that is transfected is taken from a subject. In some embodiments, the cell is derived from cells taken from a subject, such as a cell line. A wide variety of cell lines for tissue culture are known in the art. Examples of cell lines include, but are not limited to, C8161, CCRF-CEM, MOLT, mIMCD-3, NHDF, HeLa-S3, Huh1, Huh4, Huh7, HUVEC, HASMC, HEKn, HEKa, MiaPaCell, Panc1, PC-3, TF1, CTLL-2, C1R, Rat6, CV1, RPTE, A10, T24, J82, A375, ARH-77, Calu1, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRC5, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A 172, A20, A253, A431, A-549, ALC, B16, B35, BCP-1 cells, BEAS-2B, bEnd.3, BHK-21, BR 293. BxPC3. C3H-10T1/2, C6/36, Cal-27, CHO, CHO-7, CHO-IR, CHO-K1, CHO-K2, CHO-T, CHO Dhfr −/−, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepa1c1c7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK 11, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof.

Cell lines are available from a variety of sources known to those with skill in the art (see, e.g., the American Type Culture Collection (ATCC) (Manassas, Va.)). In some embodiments, a cell transfected with one or more vectors described herein is used to establish a new cell line comprising one or more vector-derived sequences. In some embodiments, a cell transiently transfected with the components of a CRISPR system as described herein (such as by transient transfection of one or more vectors, or transfection with RNA), and modified through the activity of a CRISPR complex, is used to establish a new cell line comprising cells containing the modification but lacking any other exogenous sequence. In some embodiments, cells transiently or non-transiently transfected with one or more vectors described herein, or cell lines derived from such cells are used in assessing one or more test nucleic acids and/or proteins.

The term “immunoprecipitating” or “immunoprecipitation” refers to affinity purification of an antigen using an antibody.

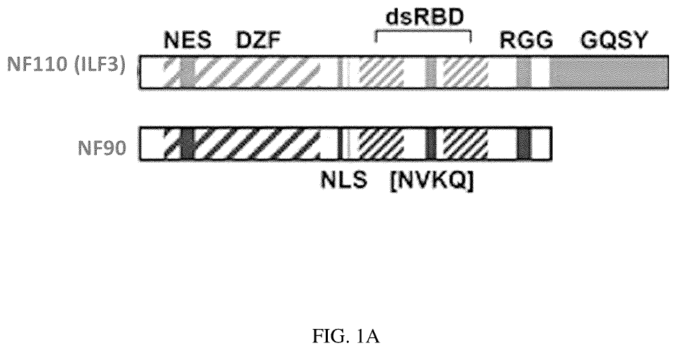

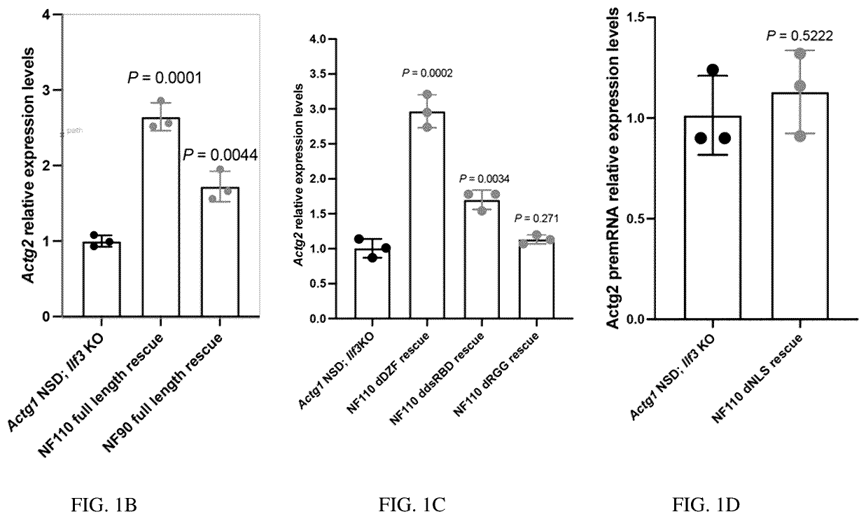

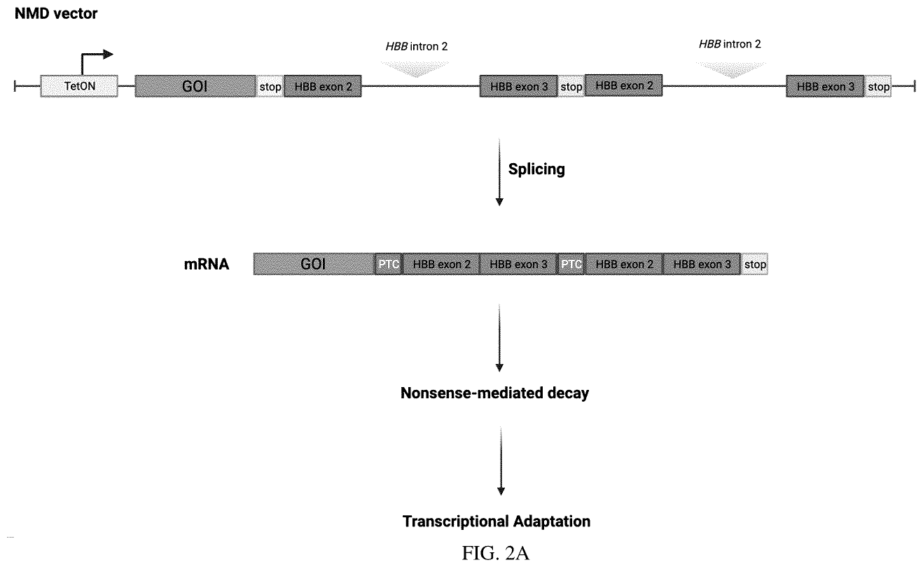

The term “interleukin enhancer-binding factor 3” or “ILF3” refers to an RNA-binding protein that has been implicated as a transcription factor and a negative regulator of innate immune responses and dendritic cell maturation. Naturally occurring ILF3 exists in at least two isoforms (NF110 and NF90). The NF110 isoform comprises the following domains: nuclear export signal (NES), domain associated with zinc finger (DZF), double-stranded RNA-binding domain 1 (dsRBD1), double-stranded RNA-binding domain 2 (dsRBD2), RGG-repeat motif, GQSY-repeat motif (GQSY-repeat or GQSY motif), and nuclear localization signal (NLS). The NF90 isoform comprises the following domains: NES, DZF, NLS, dsRBD1, dsRBD2, and RGG-repeat motif. In some embodiments, an isoform of ILF3 further comprises an NVKQ motif (NVKQ). See, e.g., FIG. 1A, Nazitto et al., J. Immunol. 2021 Jun. 15; 206(12): 2949-2965, and Reichman et al., J. Mol. Biol. 2003 Sep. 5; 332(1):85-98.

The term “interleukin enhancer-binding factor 3 sequence” or “ILF3 sequence” as used in this disclosure refers to a protein comprising one or more of the following domains: a GQSY-repeat motif, double-stranded RNA-binding domain 1 (dsRBD1) domain, double-stranded RNA-binding domain 2 (dsRBD2) domain, a nuclear localization signal (NLS), a RGG-repeat motif, an NES domain, a DZF domain, and/or a NVKQ motif (NVKQ). In some embodiments, an ILF3 sequence comprises: double-stranded RNA-binding domain 1 (dsRBD1) domain, double-stranded RNA-binding domain 2 (dsRBD2) domain, a nuclear localization signal (NLS), and a RGG-repeat motif. In some embodiments, an ILF3 sequence comprises: a GQSY-repeat motif, double-stranded RNA-binding domain 1 (dsRBD1) domain, double-stranded RNA-binding domain 2 (dsRBD2) domain, a nuclear localization signal (NLS), and a RGG-repeat motif. In some embodiments, an ILF3 sequence comprises a deletion of one or more of the following domains relative to wild-type ILF3: double-stranded RNA-binding domain 1 (dsRBD1) domain, double-stranded RNA-binding domain 2 (dsRBD2) domain, a nuclear localization signal (NLS), a RGG-repeat motif, an NES domain, a DZF domain, and/or a NVKQ motif (NVKQ). In some embodiments, an ILF3 sequence comprises a deletion of a GQSY-repeat motif relative to wild-type ILF3. In some embodiments, a wild-type ILF3 comprises the amino acid sequence set forth in any one of SEQ ID NOs: 1-4. In some embodiments, an ILF3 sequence comprises one or more domains shown in FIG. 1A. Any of the ILF3 domains or motifs disclosed herein may be mutated relative to a wild-type ILF3 domain or motif.

A “linker” as used herein refers is an organic molecule, group, polymer, or chemical moiety that adjoins two domains. The linker can be an amino acid sequence in the case of a linker joining two fusion proteins. For example, a linker may be an XTEN80 linker. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated. See also, e.g., Chen et al., Adv Drug Deliv Rev. 2013 October; 65(10):1357-69.

The term “lipid nanoparticle” or “LNP” refers to spherical vesicle made at least in part of ionizable lipids. The diameter of lipid nanoparticle varies and ranges between 10 and 1000 nanometers. The core of a lipid nanoparticle comprises a matrix of solubilized lipid molecules and is stabilized by surfactants. The compositions of lipid nanoparticles vary depending on the therapeutic purpose. Examples of components, formulations, and applications of lipid nanoparticles may be found in Hou et al., Lipid nanoparticles for mRNA delivery. Nature Rev Mat. 6:1078-1094 (2021).

The term “mRNA” or “mRNA molecule” refers to messenger RNA, or the RNA that serves as a template for protein synthesis in a cell. The sequence of a strand of mRNA is based on the sequence of a complementary strand of DNA comprising a sequence coding for the protein to be synthesized.

The term “mutation” or “mutated” as used herein, refers to a substitution of a residue within a sequence, e.g., a nucleic acid or amino acid sequence, with another residue; a deletion or insertion of one or more residues within a sequence; or a substitution of a residue within a sequence, e.g., within a genome in a cell or subject. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4^{th} ed., Cold Spring Harbor Laboratory Press, Cold Spring Harbor, N.Y. (2012)). Mutations can include a variety of categories, such as single base polymorphisms, microduplication regions, indel, and inversions, and is not meant to be limiting in any way. Mutations can include “loss-of-function” mutations which is the normal result of a mutation that reduces or abolishes a protein activity. Most loss-of-function mutations are recessive, because in a heterozygote the second chromosome copy carries an unmutated version of the gene coding for a fully functional protein whose presence compensates for the effect of the mutation. There are some exceptions where a loss-of-function mutation is dominant, one example being haploinsufficiency, where the organism is unable to tolerate the approximately 50% reduction in protein activity suffered by the heterozygote. This is the explanation for a few genetic diseases in humans, including Marfan syndrome which results from a mutation in the gene for the connective tissue protein fibrillin. Mutations also embrace “gain-of-function” mutations, which is one which confers an abnormal activity on a protein or cell that is otherwise not present in a normal condition. Many gain-of-function mutations are in regulatory sequences rather than in coding regions, and can therefore have a number of consequences. For example, a mutation might lead to one or more genes being expressed in the wrong tissues, these tissues gaining functions that they normally lack. Alternatively, the mutation could lead to overexpression of one or more genes involved in control of the cell cycle, thus leading to uncontrolled cell division and hence to cancer. Because of their nature, gain-of-function mutations are usually dominant.

A “nuclear localization signal,” “nuclear localization signal sequence” or “NLS” refers to an amino acid sequence which helps promote translocation of a protein into the cell nucleus. Such sequences are well-known in the art. The nucleotide sequence encoding an NLS is “operably linked” to the nucleotide sequence encoding a protein to which the NLS is fused when two coding sequences are “in-frame with each other” and are translated as a single polypeptide fusing two sequences. The fusion proteins described herein may comprise any known NLS sequence, including any of those described in Cokol et al., “Finding nuclear localization signals,” EMBO Rep., 2000, 1(5): 411-415 and Freitas et al., “Mechanisms and Signals for the Nuclear Import of Proteins,” Current Genomics, 2009, 10(8): 550-7, Plank et al., International PCT application PCT/EP2000/011690, filed Nov. 23, 2000, published as WO/2001/038547 on May 31, 2001, the contents of which are incorporated herein by reference.

Cells that may contain any of the compositions described herein include prokaryotic cells and eukaryotic cells. In some embodiments, the cell is in vitro (e.g., cultured cell). In some embodiments, the cell is in vivo (e.g., in a subject such as a human subject). In some embodiments, the cell is ex vivo (e.g., isolated from a subject and may be administered back to the same or a different subject).

The term “nuclease” refers to an enzyme which cleaves or degrades nucleic acids. Exemplary nucleases include but are not limited to endonucleases, exonucleases, and ribonucleases.

The terms “nucleic acid”, “nucleic acid molecule”, “ribonucleotide”, “polynucleotide”, “nucleotide sequence”, “nucleic acid sequence”, and “oligonucleotide” refer to a single nucleotide or a series of nucleotide bases (also called “nucleotides”) in DNA and RNA. The polynucleotides can be chimeric mixtures or derivatives or modified versions thereof, single-stranded or double-stranded. The oligonucleotide can be modified at the base moiety, sugar moiety, or phosphate backbone, for example, to improve stability of the molecule, its hybridization parameters, etc. An oligonucleotide may comprise a modified base moiety which is selected from the group including, but not limited to, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid, 5-methyl-2-thiouracil, 3-(3-amino-3-N-2-carboxypropyl) uracil, a thio-guanine, and 2,6-diaminopurine. A nucleotide sequence typically carries genetic information, including the information used by cellular machinery to make proteins and enzymes. These terms include double- or single-stranded genomic and cDNA, RNA, any synthetic and genetically manipulated polynucleotide, and both sense and antisense polynucleotides. This includes single- and double-stranded molecules, i.e., DNA-DNA, DNA-RNA and RNA-RNA hybrids, as well as “protein nucleic acids” (PNAs) formed by conjugating bases to an amino acid backbone. This also includes nucleic acids containing carbohydrate or lipids. Exemplary DNAs include single-stranded DNA (ssDNA), double-stranded DNA (dsDNA), plasmid DNA (pDNA), genomic DNA (gDNA), complementary DNA (cDNA), antisense DNA, chloroplast DNA (ctDNA or cpDNA), microsatellite DNA, mitochondrial DNA (mtDNA or mDNA), kinetoplast DNA (kDNA), provirus, lysogen, repetitive DNA, satellite DNA, and viral DNA. Exemplary RNAs include single-stranded RNA (ssRNA), double-stranded RNA (dsRNA), small interfering RNA (siRNA), messenger RNA (mRNA), precursor messenger RNA (pre-mRNA), small hairpin RNA or short hairpin RNA (shRNA), microRNA (miRNA), guide RNA (gRNA), transfer RNA (tRNA), antisense RNA (asRNA), heterogeneous nuclear RNA (hnRNA), coding RNA, non-coding RNA (ncRNA), long non-coding RNA (long ncRNA or lncRNA), satellite RNA, viral satellite RNA, signal recognition particle RNA, small cytoplasmic RNA, small nuclear RNA (snRNA), ribosomal RNA (rRNA), Piwi-interacting RNA (piRNA), polyinosinic acid, ribozyme, flexizyme, small nucleolar RNA (snoRNA), spliced leader RNA, viral RNA, and viral satellite RNA.

Polynucleotides described herein may be synthesized by standard methods known in the art, e.g., by use of an automated DNA synthesizer (such as those that are commercially available from Biosearch, Applied Biosystems, etc.). As examples, phosphorothioate oligonucleotides may be synthesized by the method of Stein et al., Nucl. Acids Res., 16, 3209, (1988), methylphosphonate oligonucleotides can be prepared by use of controlled pore glass polymer supports (Sarin et al., Proc. Natl. Acad. Sci. U.S.A. 85, 7448-7451, (1988)). A number of methods have been developed for delivering antisense DNA or RNA to cells, e.g., antisense molecules can be injected directly into the tissue site, or modified antisense molecules, designed to target the desired cells (antisense linked to peptides or antibodies that specifically bind receptors or antigens expressed on the target cell surface) can be administered systemically. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines. In some embodiments, a recombinant DNA construct in which the antisense oligonucleotide is placed under the control of a strong promoter. In some embodiments, the use of such a construct to transfect target cells in the patient will result in the transcription of sufficient amounts of single stranded RNAs. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human, cells. Such promoters can be inducible or constitutive. Any type of plasmid, cosmid, yeast artificial chromosome, or viral vector can be used to prepare the recombinant DNA construct that can be introduced directly into the tissue site.

The polynucleotides may be flanked by natural regulatory (expression control) sequences or may be associated with heterologous sequences, including promoters, internal ribosome entry sites (IRES) and other ribosome binding site sequences, enhancers, response elements, suppressors, signal sequences, polyadenylation sequences, introns, 5′- and 3′-non-coding regions, and the like. The nucleic acids may also be modified by many means known in the art. Non-limiting examples of such modifications include methylation, “caps”, substitution of one or more of the naturally occurring nucleotides with an analog, and internucleotide modifications, such as, for example, those with uncharged linkages (e.g., methyl phosphonates, phosphotriesters, phosphoroamidates, carbamates, etc.) and with charged linkages (e.g., phosphorothioates, phosphorodithioates, etc.). Polynucleotides may contain one or more additional covalently linked moieties, such as, for example, proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.), and alkylators. The polynucleotides may be derivatized by formation of a methyl or ethyl phosphotriester or an alkyl phosphoramidate linkage. Furthermore, the polynucleotides herein may also be modified with a label capable of providing a detectable signal, either directly or indirectly. Exemplary labels include radioisotopes, fluorescent molecules, isotopes (e.g., radioactive isotopes), biotin, and the like.

The term “paralog,” as used herein, refers to a gene that arises from duplication of another gene within a genome of a species.

A “promoter” refers to a control region of a nucleic acid sequence at which initiation and rate of transcription of the remainder of a nucleic acid sequence are controlled. A promoter may also contain sub-regions at which regulatory proteins and molecules may bind, such as RNA polymerase and other transcription factors. Promoters may be constitutive, inducible, activatable, repressible, tissue-specific, or any combination thereof. A promoter drives expression or drives transcription of the nucleic acid sequence that it regulates. Herein, a promoter is considered to be “operably linked” when it is in a correct functional location and orientation in relation to a nucleic acid sequence it regulates to control (“drive”) transcriptional initiation and/or expression of that sequence.

A promoter may be one naturally associated with a gene or sequence, as may be obtained by isolating the 5′ non-coding sequences located upstream of the coding segment of a given gene or sequence. Such a promoter is referred to as an “endogenous promoter.” In some embodiments, a coding nucleic acid sequence may be positioned under the control of a recombinant or heterologous promoter, which refers to a promoter that is not normally associated with the encoded sequence in its natural environment. Such promoters may include promoters of other genes; promoters isolated from any other cell; and synthetic promoters or enhancers that are not “naturally occurring” such as, for example, those that contain different elements of different transcriptional regulatory regions and/or mutations that alter expression through methods of genetic engineering that are known in the art. In addition to producing nucleic acid sequences of promoters and enhancers synthetically, sequences may be produced using recombinant cloning and/or nucleic acid amplification technology, including polymerase chain reaction (PCR).

In some embodiments, the promoter sequence comprises a mammalian promoter. In some embodiments, the promoter sequence is a SV40 promoter, a CMV promoter, a UBC promoter, an EF1A promoter, a PGK promoter, or a CAG promoter.

In some embodiments, promoters used in accordance with the present disclosure are “inducible promoters,” which are promoters that are characterized by regulating (e.g., initiating or activating) transcriptional activity when in the presence of, influenced by or contacted by an inducer signal. An inducer signal may be endogenous or a normally exogenous condition (e.g., light), compound (e.g., chemical or non-chemical compound) or protein that contacts an inducible promoter in such a way as to be active in regulating transcriptional activity from the inducible promoter. Thus, a “signal that regulates transcription” of a nucleic acid refers to an inducer signal that acts on an inducible promoter. A signal that regulates transcription may activate or inactivate transcription, depending on the regulatory system used. Activation of transcription may involve directly acting on a promoter to drive transcription or indirectly acting on a promoter by inactivation a repressor that is preventing the promoter from driving transcription. Conversely, deactivation of transcription may involve directly acting on a promoter to prevent transcription or indirectly acting on a promoter by activating a repressor that then acts on the promoter.

An inducible promoter may be regulated in vivo by a chemical agent, temperature, or light, for example. Inducible promoters enable, for example, temporal and/or spatial control of gene expression. Inducible promoters for use in accordance with the present disclosure include any inducible promoter described herein or known to one of ordinary skill in the art. Examples of inducible promoters include, without limitation, chemically/biochemically-regulated and physically-regulated promoters such as alcohol-regulated promoters, tetracycline-regulated promoters (e.g., anhydrotetracycline (aTc)-responsive promoters and other tetracycline responsive promoter systems, which include a tetracycline repressor protein (tetR), a tetracycline operator sequence (tetO) and a tetracycline transactivator fusion protein (tTA)), steroid-regulated promoters (e.g., promoters based on the rat glucocorticoid receptor, human estrogen receptor, moth ecdysone receptors, and promoters from the steroid/retinoid/thyroid 25 receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), and light-regulated promoters (e.g., light responsive promoters from plant cells).

A “protein,” “peptide,” or “polypeptide” comprises a polymer of amino acid residues linked together by peptide bonds. The term refers to proteins, polypeptides, and peptides of any size, structure, or function. Typically, a protein will be at least three amino acids long. A protein may refer to an individual protein or a collection of proteins. Inventive proteins preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in a protein may be modified, for example, by the addition of a chemical entity such as a carbohydrate group, a hydroxyl group, a phosphate group, a farnesyl group, an isofarnesyl group, a fatty acid group, a linker for conjugation or functionalization, or other modification. A protein may also be a single molecule or may be a multi-molecular complex. A protein may be a fragment of a naturally occurring protein or peptide. A protein may be naturally occurring, recombinant, synthetic, or any combination of these.

Such manipulation may be done to replace a codon with a redundant codon encoding the same or a conservative amino acid, while typically introducing or removing a sequence recognition site. Alternatively, it may be performed to join together nucleic acid segments of desired functions to generate a single genetic entity comprising a desired combination of functions not found in nature. Restriction enzyme recognition sites are often the target of such artificial manipulations, but other site specific targets, e.g., promoters, DNA replication sites, regulation sequences, control sequences, open reading frames, or other useful features may be incorporated by design.

The term “ribonucleoprotein complex” refers to a complex comprising a ribonucleic acid and an RNA-binding protein.

The term “RNA-binding protein” refers to a protein that is capable of binding to a ribonucleic acid. In some embodiments, a RNA-binding protein comprises a double stranded RNA binding domain. In some embodiments, a RNA-binding protein comprises a single-stranded RNA binding domain. In some embodiments, a RNA-binding protein comprises both a single-stranded RNA binding domain. An RNA-binding protein may comprise one or more protein-protein interaction domains. In some embodiments, a RNA-binding protein is a fusion protein comprising a Cas protein and a ILF3 sequence disclosed herein.

The term “RNA decay” or “ribonucleic acid decay” refers to degradation of an mRNA transcript. Cells often use RNA decay pathways to detect and degrade aberrant mRNA transcripts. For example, nonsense-mediated decay is a surveillance pathway used by cells to eliminate and/or degrade mRNA transcripts that comprise one or more premature stop codons (PTC). See, e.g., Kurosaki et al., Nat Rev Mol Cell Biol. 2019 July; 20(7):406-420. The No-Go Decay (NGD) mRNA surveillance pathway degrades mRNAs that have stalled ribosomes. Ribosomes may be stalled by a secondary structure that forms in the RNA. For example, an mRNA transcript may have sequences that are complementary to one another such that the complementary sequences hybridize to form a secondary structure. See, e.g., Doma et al. Nature 440, 561-564 (2006) and Pasos et al., Mol. Biol. Cell 20, 3025-3032 (2009). The non-stop decay or no-stop decay pathway detects and degrades mRNA transcripts that lack a proper stop codon. Such aberrant transcripts are detected during translation when the ribosome translates into the polyA tail and stalls. See, e.g., Wiley Interdiscip Rev RNA. 2010 July-August; 1(1):132-41 and Navickas et al., Nat Commun. 2020 Jan. 8; 11(1):122. The poly(A) sequence or poly(A) tail is a chain of two or more adenine nucleotides. A poly(A) tail is often added to a mRNA molecule during RNA processing. In some instances, a poly(A) tail is 2, 3, 4, 5, 6, 7, 8, 9, 10, 15, 20, 30, 40, 50, 60, 70, 80, 90, 100, 200, 300, 400, or 500 nucleotides in length, including any values in-between.

The term “RNA-targeting Cas protein” refers to a Cas protein that when associated with crRNA recognizes ribonucleic acid target sequences. Non-limiting examples of RNA-targeting Cas proteins include Type II Cas proteins, Type III Cas proteins, Type VI Cas proteins, and Cas7-11. In some embodiments, a Type III Cas protein is a Csm protein. In some embodiments, a Type III Cas protein is a Cmr protein. In some embodiments, a Type VI Cas protein is a Cas13 protein. See also, e.g., Burmistrz et al., Int J Mol Sci. 2020 February; 21(3): 1122.

“RNA transcript” refers to the product resulting from RNA polymerase-catalyzed transcription of a DNA sequence. When the RNA transcript is a complementary copy of the DNA sequence, it is referred to as the primary transcript. An RNA transcript may be a sense transcript, which may be used as a template for translation. An RNA transcript may be an antisense transcript, which is complementary to the sense transcript. In some embodiments, an RNA transcript is a protein coding messenger RNA (mRNA) or it may be an RNA sequence derived from post-transcriptional processing of the primary transcript and is referred to as the mature RNA. “Messenger RNA (mRNA)” refers to the RNA that is without introns and can be translated into polypeptides by the cell. “cRNA” refers to complementary RNA, transcribed from a recombinant cDNA template. “cDNA” refers to DNA that is complementary to and derived from an mRNA template. The cDNA can be single-stranded or converted to double-stranded form using, for example, the Klenow fragment of DNA polymerase I.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease.

The term “transcriptional adaptation (TA)” refers to a cellular mechanism by which mutations that cause mutant mRNA degradation trigger the transcriptional modulation of another gene, which may be referred to as an adapting gene. As a non-limiting example, degradation of mutant mRNA of a first gene can lead to increased expression levels of one or more second genes exhibiting sequence similarity with the mutated gene's mRNA. The first gene may be referred herein as a perturbed gene. The second gene may be referred to herein as an adapting gene. Non-limiting examples of perturbed gene-adapting gene pairs are provided in Table 7.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence.

The term “trigger deoxyribonucleic acid” or “trigger DNA” is used to refer to an engineered deoxyribonucleic acid that is capable of increasing the expression of a gene of interest in which the engineered deoxyribonucleic acid is shorter than a mRNA sequence encoding the gene of interest.

The term “trigger nucleic acid” is used to refer to an engineered nucleic acid that is capable of increasing the expression of a gene of interest in which the engineered nucleic acid sequence is shorter than a mRNA transcript encoding the gene of interest.

The term “trigger RNA” is used to refer to an engineered ribonucleic acid (RNA) that is capable of increasing the expression of a gene of interest in which the ribonucleic acid sequence is shorter than a mRNA sequence encoding the gene of interest. In some instances, a trigger RNA is complementary to one or more regions of an antisense transcript.

The term “tumor suppressor” is used to refer to a protein that inhibits the cell cycle and/or promote apoptosis and/or otherwise inhibits the development, growth, or progression of cancer. Non-limiting examples of tumor suppressor genes encoding a tumor suppressor include genes encoding p53, RB, p16, BRCA1, p14, and DNA mismatch repair protein 2 (MSH2).

A “vector,” “expression vector,” or “viral vector” as used herein refers to a nucleic acid that can be modified to encode a gene of interest and that is able to enter into a host cell, mutate and replicate within the host cell, and then transfer a replicated form of the vector into another host cell. Exemplary suitable vectors include viral vectors, such as retroviral vectors or bacteriophages and filamentous phage, and conjugative plasmids. Additional suitable vectors will be apparent to those of skill in the art based on the instant disclosure. In some embodiments, a vector disclosed herein further encodes a selection marker. Non-limiting examples of selection markers include puromycin, blasticidin, geneticin, hygromycin B, mycophenolic acid, and zeocin.

Other than in the examples, or where otherwise indicated, all numbers expressing quantities of ingredients or reaction conditions used herein should be understood as modified in all instances by the term “about.” “About” and “approximately” shall generally mean an acceptable degree of error for the quantity measured given the nature or precision of the measurements. Exemplary degrees of error are within 20 percent (%), typically, within 10%, or more typically, within 5%, 4%, 3%, 2%, or 1% of a given value or range of values.

Unless otherwise required by context, singular terms shall include pluralities, and plural terms shall include the singular.

### DETAILED DESCRIPTION OF CERTAIN EMBODIMENTS

Provided herein, in some aspects, are fusion proteins, RNA-binding proteins, nucleic acids, complexes, and compositions thereof for upregulating gene expression, which may be useful in treating diseases characterized by downregulation of expression of one or more genes. Systems and methods for identifying engineered nucleic acids capable of upregulating gene expression are also provided.

### Interleukin Enhancer Binding Factor 3 (ILF3)

Transcriptional adaptation (TA) is a recently described phenomenon through which mutant mRNA decay modulates the expression of genes exhibiting sequence similarity. See, e.g., El-Brolosy et al., Nature. 2019 April; 568(7751):193-197. According to the proposed model, mRNA degradation generates short mRNA fragments that act as guide RNAs to recruit a RNA-binding protein (RBP) to loci of genes exhibiting sequence similarity (as paralogs or the mutated gene itself) through homology-mediated base pairing which then helps promote gene expression by recruiting transcription factors (TFs) and chromatin remodelers (CRs) and/or repressing antisense RNAs to allow for derepression of the sense RNA. The modulated genes are referred to as adapting genes. This disclosure is based in part on the finding that Interleukin enhancer binding factor 3 (ILF3) is an RNA-binding protein that mediates transcriptional adaptation. Without wishing to be bound by any particular theory, mRNA decay intermediates may guide ILF3 to genes exhibiting sequence similarity by hybridizing to antisense RNAs of the genes. Upon its recruitment, ILF3 may recruit transcription factors and chromatin remodelers, e.g., the COMPASS complex, PRMT1, BRG1, WDR5, and YY1, to help promote gene expression.

ILF3 has been implicated as a transcription factor and a negative regulator of innate immune responses and dendritic cell maturation. Naturally occurring ILF3 exists as at least two isoforms (NF110 and NF90). The NF110 isoform comprises the following domains: nuclear export signal (NES), domain associated with zinc finger (DZF), double-stranded RNA-binding domain 1 (dsRBD1), double-stranded RNA-binding domain 2 (dsRBD2), RGG-repeat motif, GQSY-repeat motif (GQSY-repeat or GQSY motif), and nuclear localization signal (NLS). The NF90 isoform comprises the following domains: NES, DZF, NLS, dsRBD1, dsRBD2, and RGG-repeat motif. In some embodiments, an isoform of ILF3 further comprises an NVKQ motif (NVKQ). See, e.g., FIG. 1A, Nazitto et al., J Immunol. 2021 Jun. 15; 206(12): 2949-2965, and Reichman et al., J Mol Biol. 2003 Sep. 5; 332(1):85-98.

The ILF3 sequences used in the compositions and methods of the present disclosure comprise one or more of the following: a GQSY-repeat motif, double-stranded RNA-binding domain 1 (dsRBD1) domain, double-stranded RNA-binding domain 2 (dsRBD2) domain, a nuclear localization signal (NLS), a RGG-repeat motif, an NES domain, a DZF domain, and/or a NVKQ motif (NVKQ). In some embodiments, an ILF3 sequence comprises double-stranded RNA-binding domain 1 (dsRBD1) domain, double-stranded RNA-binding domain 2 (dsRBD2) domain, a nuclear localization signal (NLS), and a RGG-repeat motif. In some embodiments, an ILF3 sequence comprises: a GQSY-repeat motif, double-stranded RNA-binding domain 1 (dsRBD1) domain, double-stranded RNA-binding domain 2 (dsRBD2) domain, a nuclear localization signal (NLS), and a RGG-repeat motif. In some embodiments, an ILF3 sequence comprises a deletion of one or more of the following domains relative to wild-type ILF3: double-stranded RNA-binding domain 1 (dsRBD1) domain, double-stranded RNA-binding domain 2 (dsRBD2) domain, a nuclear localization signal (NLS), a RGG-repeat motif, an NES domain, a DZF domain, and/or a NVKQ motif (NVKQ). In some embodiments, an ILF3 sequence comprises a deletion of a GQSY-repeat motif relative to wild-type ILF3. In some embodiments, a wild-type ILF3 comprises the amino acid sequence set forth in any one of SEQ ID NOs: 1-4. In some embodiments, an ILF3 sequence comprises one or more domains shown in FIG. 1A. Any of the ILF3 domains or motifs disclosed herein may be mutated relative to a wild-type ILF3 domain or motif.

In some embodiments, an ILF3 protein comprises an amino acid sequence that is at least 70% (e.g., at least 75%, 80%, 85%, 90%, 95%, 98%, 99%, or 100%) identical to human NF110 isoform of ILF3 (SEQ ID NO: 1), human NF90 isoform of ILF3 (SEQ ID NO: 2), mouse NF110 isoform of ILF3 (SEQ ID NO: 3), and/or mouse NF90 isoform of ILF3 (SEQ ID NO: 4).

In some embodiments, an ILF3 protein comprises a nuclear localization sequence (NLS). In some embodiments, a NLS of the ILF3 protein comprises an amino acid sequence that is at least 70% (e.g., at least 75%, 80%, 85%, 90%, 95%, 98%, 99%, or 100%) identical to SEQ ID NO: 6.

An ILF3 sequence may comprise the amino acid sequence NVKQ (SEQ ID NO: 7) or it may not. The NVKQ may act as an activator of the ILF3 sequence. See, e.g., Reichman et al., J Mol Biol. 2003 Sep. 5; 332(1):85-98. In some embodiments, an ILF3 sequence does not comprise SEQ ID NO: 7.

Double-stranded RNA-binding domains (dsRBDs) help proteins recognize double-stranded RNA (dsRNA) and related structures. In some embodiments, a dsRB1 domain of the ILF3 protein comprises an amino acid sequence that is at least 70% (e.g., at least 75%, 80%, 85%, 90%, 95%, 98%, 99%, or 100%) identical to SEQ ID NO: 8 or 12. In some embodiments, a dsRBD2 domain of the ILF3 protein comprises an amino acid sequence that is at least 70% (e.g., at least 75%, 80%, 85%, 90%, 95%, 98%, 99%, or 100%) identical to SEQ ID NO: 10.

Domain associated with zinc fingers (DZF) is implicated in allowing proteins to heterodimerize. In some embodiments, a DZF domain of the ILF3 protein comprises an amino acid sequence that is at least 70% (e.g., at least 75%, 80%, 85%, 90%, 95%, 98%, 99%, or 100%) identical to SEQ ID NO: 9 or 13.

The arginine-glycine-glycine (RGG) domain has been implicated in binding to nucleic acid. In some embodiments, a RGG domain of the ILF3 protein comprises an amino acid sequence that is at least 70% (e.g., at least 75%, 80%, 85%, 90%, 95%, 98%, 99%, or 100%) identical to SEQ ID NO: 11 or 14.

The GQSY domain has been implicated in interacting with nucleic acids. In some embodiments, a GQSY domain of the ILF3 protein comprises an amino acid sequence that is at least 70% (e.g., at least 75%, 80%, 85%, 90%, 95%, 98%, 99%, or 100%) identical to SEQ ID NO: 61 or 69.

The term “interleukin enhancer-binding factor 3 sequence” or “ILF3 sequence” as used in this disclosure refers to a protein comprising one or more of the following: a GQSY-repeat motif, double-stranded RNA-binding domain 1 (dsRBD1) domain, double-stranded RNA-binding domain 2 (dsRBD2) domain, a nuclear localization signal (NLS), a RGG-repeat motif, an NES domain, a DZF domain, and/or a NVKQ motif (NVKQ). In some embodiments, an ILF3 sequence comprises: double-stranded RNA-binding domain 1 (dsRBD1) domain, double-stranded RNA-binding domain 2 (dsRBD2) domain, a nuclear localization signal (NLS), and a RGG-repeat motif. In some embodiments, an ILF3 sequence comprises: a GQSY-repeat motif, double-stranded RNA-binding domain 1 (dsRBD1) domain, double-stranded RNA-binding domain 2 (dsRBD2) domain, a nuclear localization signal (NLS), and a RGG-repeat motif. In some embodiments, an ILF3 sequence comprises a deletion of one or more of the following domains relative to wild-type ILF3: double-stranded RNA-binding domain 1 (dsRBD1) domain, double-stranded RNA-binding domain 2 (dsRBD2) domain, a nuclear localization signal (NLS), a RGG-repeat motif, an NES domain, a DZF domain, and/or a NVKQ motif (NVKQ). In some embodiments, an ILF3 sequence comprises a deletion of a GQSY-repeat motif relative to wild-type ILF3. In some embodiments, a wild-type ILF3 comprises the amino acid sequence set forth in any one of SEQ ID NOs: 1-4. In some embodiments, an ILF3 sequence comprises one or more domains shown in FIG. 1A. Any of the ILF3 domains or motifs disclosed herein may be mutated relative to a wild-type ILF3 domain or motif. In some embodiments, an ILF3 sequence comprises one or more of the domains or motifs shown in Table 1.

In some embodiments, a ILF3 sequence comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or is 100% identical to any one of SEQ ID NOs: 1-14, 61, and 69. See, e.g., Table 1.

In some embodiments, a ILF3 sequence is not a wild-type ILF3 sequence and retains at least 25% to 100% (e.g., at least 25%, at least 50%, at least 75%, or 100%, including all values in between) of the activity of a wild-type ILF3 sequence. For example, an ILF3 sequence may comprise one or more domains that are mutated relative to a wild-type ILF3 domain. Non-limiting examples of ILF3 activity include the ability of a ILF3 sequence to bind to a ribonucleic acid and the ability of a ILF3 sequence to drive expression of a gene of interest.

Aspects of the present disclosure provide non-naturally occurring proteins comprising an ILF3 sequence. In some embodiments, an ILF3 sequence does not comprise one or more of the following domains: a double-stranded RNA-binding domain (dsRBD) domain, a nuclear localization signal (NLS), a RGG-repeat motif, an NES domain, a DZF domain, and/or a NVKQ motif (NVKQ). In some embodiments, a non-naturally occurring protein comprises an ILF3 sequence that does not comprise a GQSY-repeat motif. In some embodiments, the non-naturally occurring protein comprises an ILF3 sequence that comprises a double-stranded RNA-binding domain (dsRBD) domain, a nuclear localization signal (NLS), GQSY-repeat motif, and a RGG-repeat motif.

### RNA-Targeting Cas Proteins

Clustered Regularly Interspaced Short Palindromic Repeats (CRISPR) systems were originally identified in prokaryotes and help defend prokaryotes against mobile genetic elements from pathogens. Naturally occurring CRISPR systems comprise a CRISPR locus that comprises a series of short sequences (“spacers”) derived from a pathogen that allow for recognition of mobile genetic elements from previous infections. Repetitive regulatory sequences (“repeats”) separate the spacers. Naturally occurring Cas proteins are effectors for these prokaryotic CRISPR systems. Naturally occurring CRISPR-Cas systems undergo adaptation, maturation, and interference. New spacers are introduced into the CRISPR locus at the leader end during the adaptation phase. Then, the CRISPR array is transcribed into a transcript called the pre-CRISPR RNA (pre-crRNA), which is subsequently processed into CRISPR RNA (crRNA molecules). In some embodiments, Cas proteins process pre-crRNA into crRNA molecules. Cas proteins form ribonucleoprotein complexes with crRNAs and the ribonucleoprotein complexes recognize target nucleic acid sequences that are complementary to a sequence encoded by the crRNA. Upon binding of the Cas-crRNA complex to a target nucleic acid, naturally occurring Cas proteins will cleave the target nucleic acid.

Aspects of the present disclosure relate to RNA-targeting Cas proteins. In some embodiments, a Cas protein does not comprise nuclease activity toward target RNA. For example, a Cas protein may be able to process pre-crRNAs to make mature crRNAs but cannot cleave target RNA. In some embodiments, an RNA-targeting Cas protein does not comprise nickase activity. The RNA-targeting Cas proteins disclosed herein recognize ribonucleic acid target sequences. Non-limiting examples of RNA-targeting Cas proteins include Type II Cas proteins, Type III Cas proteins, Type VI Cas proteins, and Cas7-11.

Type II Cas proteins including Cas9 proteins typically use a trans activating RNA (tracrRNA) to interact with crRNAs. Cas9-tracrRNA-crRNA complexes typically detect protospacer-associated motifs (PAM) and crRNA hybridizes to the target DNA. Then, Cas9 subsequently cleaves the target DNA. A “protospacer adjacent motif” (PAM) is typically a sequence of nucleotides located adjacent to (e.g., within 10, 9, 8, 7, 6, 5, 4, 3, 3, or 1 nucleotide(s) of a target sequence). A PAM sequence is “immediately adjacent to” a target sequence if the PAM sequence is contiguous with the target sequence (that is, if there are no nucleotides located between the PAM sequence and the target sequence). In some embodiments, a PAM sequence is a wild-type PAM sequence. Examples of PAM sequences include, without limitation, NGG, NGR, NNGRR(T/N), NNNNGATT, NNAGAAW, NGGAG, NAAAAC, AWG, and CC. In some embodiments, a PAM sequence is obtained from Streptococcus pyogenes (e.g., NGG or NGR). In some embodiments, a PAM sequence is obtained from Staphylococcus aureus (e.g., NNGRR(T/N)). In some embodiments, a PAM sequence is obtained from Neisseria meningitidis (e.g., NNNNGATT). In some embodiments, a PAM sequence is obtained from Streptococcus thermophilus (e.g., NNAGAAW or NGGAG). In some embodiments, a PAM sequence is obtained from Treponema denticola (e.g., NAAAAC). In some embodiments, a PAM sequence is obtained from Escherichia coli (e.g., AWG). In some embodiments, a PAM sequence is obtained from Pseudomonas auruginosa (e.g., CC). Other PAM sequences are contemplated. A PAM sequence is typically located downstream (i.e., 3′) from the target sequence, although in some embodiments a PAM sequence may be located upstream (i.e., 5′) from the target sequence.

Cas9's PAM-based recognition of DNA has been used to target Cas9 to RNA. For example, PAM-presenting oligonucleotides have been used to stimulate Cas9 binding to ribonucleic acid targets. See, e.g., O'Connell et al., Nature. 2014 Dec. 11; 516(7530):263-6. Cas9 proteins have also been produced that do not require a PAM sequence to target RNA. See, e.g., Sampson et al., Nature. 2013; 497:254-257; Dugar et al., Mol. Cell. 2018; 69:893-905; Rousseau et al., Mol. Cell. 2018; 69:906-914; and Strutt et al. Elife. 2018; 7.e32724. The HNH and RuvC domains of Cas9 have been implicated as helping to mediate cleavage of target nucleic acids. In some embodiments, a Cas protein disclosed herein does not comprise a function HNH domain and/or a functional RuvC domain. In some embodiments, a Cas protein disclosed herein comprises a mutation in a HNH domain and/or RuvC domain relative to a wild-type Cas9. In some embodiments, a Cas protein disclosed herein lacks a HNH domain and/or RuvC domain relative to a wild-type Cas9. In some embodiments, a Cas9 protein comprises a mutation relative to wild-type Cas9. In some embodiments, a Cas9 protein comprises a D10A mutation and/or a H840A mutation relative to wild-type Cas9.

Type III CRISPR systems typically use Csm (Type III-A) or Cmr (type III-B) effector complexes. In some embodiments, a Cas protein is a Csm protein. In some embodiments, a Cmr protein is a Csm1, Csm3, Csm4, or Csm5 protein. In some embodiments, a Csm protein may lack nuclease activity toward a target RNA but still retain its ability to bind to RNA. See, e.g., Colognori et al., Nat Biotechnol. 2023 September; 41(9):1256-1264. In some embodiments, a Cas protein is a Cmr protein that lacks nuclease activity toward a target RNA but still retain its ability to bind to RNA. In some embodiments, a Cmr protein is a Cmr1, Cmr3, Cmr4, or Cmr6 protein.

Type VI CRISPR systems typically use Cas13. Naturally occurring Cas13 proteins are RNA endonucleases with two HEPN (higher eukaryotes and prokaryotes nucleotide-binding) domains for RNA cleavage. Cas13 proteins generally do not use a tracrRNA. In naturally occurring CRISPR systems comprising Cas13, Cas13 assembles with crRNA to recognize target RNAs and upon binding to a target RNA, Cas13 undergoes a conformation change that activates the nuclease domain of the Cas13 protein to cleave the target RNA. Cas13 proteins cleave RNA via two R-X₄-H motifs, which are characteristic features of HEPN domain. In some embodiments, a Cas protein disclosed herein comprises one or more of the following mutations relative to a wild-type Cas13d: R295A, H300A, R849A, and H854A. See also, e.g., East-Seletsky et al., Nature. 2016; 538:270-273; and Liu et al., Cell. 2017; 168:121-134.e112. In some embodiments, a Cas protein disclosed herein comprises a mutation relative to wild-type CasRx (e.g., GenBank Accession No. QMT62609.1). A non-limiting example of a mutant CasRx sequence relative to wild-type CasRx is provided as SEQ ID NO: 63. In some embodiments, a Cas13 protein is a Cas13a, Cas13b, Cas13C, Cas13d, or Cas13bt protein. See also, e.g., Cox et al., Science. 2017 Nov. 24; 358(6366): 1019-1027. In some embodiments, the Cas13 proteins for use in this disclosure does not cleave RNA target sequences. For example, a Cas13 protein for use herein may lack one or more HEPN domains and/or comprise one or more mutations in a HEPN domain that inactivates the nuclease activity of the Cas13 protein. Without being bound by a particular theory, HEPN domains in Cas13 proteins may help process pre-crRNAs and help cleave target RNA. However, mutations in one or more HEPN domains can be made to produce a Cas protein that is catalytically inactive in cleaving target RNA without affecting the Cas protein's ability to process pre-crRNA.

In some embodiments, a Cas13 protein comprises an amino acid sequence that is at least 70% (e.g., at least 75%, 80%, 85%, 90%, 95%, 98%, 99%, or 100%) identical to SEQ ID NO: 63. In some embodiments, a HEPN domain comprises an amino acid sequence that is at least 70% (e.g., at least 75%, 80%, 85%, 90%, 95%, 98%, 99%, or 100%) identical to SEQ ID NO: 64 or 65. In some embodiments, a Cas13 protein disclosed herein does not comprise the amino acid sequence set forth in SEQ ID NO: 64 and/or does not comprise the amino acid sequence set forth in SEQ ID NO: 65. In some embodiments, a Cas13 protein disclosed herein comprises one or more mutations relative to the amino acid sequence set forth in SEQ ID NO: 64 and/or one or more mutations relative to the amino acid sequence set forth in SEQ ID NO: 65. In some embodiments, a HEPN domain comprises an amino acid sequence that is at least 70% (e.g., at least 75%, 80%, 85%, 90%, 95%, 98%, 99%, or 100%) identical to SEQ ID NO: 80 or 81.

In some embodiments, a Cas protein is a Cas7-11 protein. See, e.g., Ozcan et al., Nature. 2021 September; 597(7878):720-725. In some embodiments, the Cas7-11 protein does not comprise nuclease activity toward a target RNA. In some embodiments, the Cas7-11 protein does not comprise nickase activity.

In some embodiments, a Cas protein is not a wild-type Cas protein and retains at least 25% to 100% (e.g., at least 25%, at least 50%, at least 75%, or 100%, including all values in between) of the activity of a wild-type Cas protein. Non-limiting examples of Cas activity include (1) the ability of a Cas protein to bind to a crRNA, tracrRNA, guide RNA, and/or target nucleic acid (e.g., RNA), (2) nuclease activity, and/or (3) nickase activity.

A CRISPR system may further comprise a guide RNA that comprises an engineered nucleic acid sequence that is complementary to a target RNA of interest. In some embodiments, the target RNA sequence of interest is an antisense RNA. In some embodiments, the guide RNA is about 15-120 nucleotides long and comprises a sequence of at least 10 contiguous nucleotides that is complementary to a target sequence. In some embodiments, the guide RNA is 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 97, 98, 99, 100, 101, 102, 103, 104, 105, 106, 107, 108, 109, 110, 111, 112, 113, 114, 115, 116, 117, 118, 119, or 120 nucleotides long. In some embodiments, the guide RNA comprises a sequence of 10, 11, 12, 13, 14, 15, 16, 17, 18, 19, 20, or more contiguous nucleotides that is complementary to a target sequence. In some embodiments, a target sequence encodes a protein whose activity and/or expression is downregulated in a disease. In some embodiments, a target sequence encodes an intron of a gene of interest. In some embodiments, a target sequence encodes an exon of a gene of interest. In some embodiments, a target sequence is an antisense transcript or a portion thereof. In some embodiments, a target sequence is a sense transcript or a portion thereof. In some embodiments, a target sequence encodes ACTG1, ACTG2, CDK9, REL, BDNF, or SOX9.

In some embodiments, a guide RNA comprises a sequence that is at least 70% (e.g., at least 75%, 80%, 85%, 90%, 95%, 98%, 99%, or 100%) identical to any one of SEQ ID NOs: 15-34 or to a guide RNA disclosed herein.

### Cas-ILF3 Fusion Proteins

Aspects of the present disclosure provide fusion proteins comprising an RNA-targeting Cas protein and an ILF3 sequence, which may be useful for increasing gene expression in a target-specific manner. In some embodiments, a Cas protein does not comprise nuclease activity toward target RNA. For example, a Cas protein may be able to process pre-crRNAs to make mature crRNAs but cannot cleave target RNA. In some embodiments, a Cas protein may be located at the N-terminal portion of the fusion protein relative to an ILF3 sequence. In other embodiments, an ILF3 sequence is located at the N-terminal portion of the fusion protein relative to the Cas protein. In some embodiments, a Cas protein is associated with a guide RNA.

One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. A protein may comprise different domains, for example, one or more domains of a interleukin enhancer-binding factor 3 and/or one or more domains of a Cas protein. In some embodiments, a linker is a peptide linker. For example, the linker can be an amino acid sequence in the case of a linker joining two proteins. For example, a linker may be an XTEN80 linker. In some embodiments, an XTEN80 linker comprises an amino acid sequence that is at least 70% (e.g., at least 75%, 80%, 85%, 90%, 95%, 98%, 99%, or 100%) identical to SEQ ID NO: 66. See also, e.g., Chen et al., Adv Drug Deliv Rev. 2013 October; 65(10):1357-69.

In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated.

In some embodiments, a fusion protein comprises one or more affinity tags. In some embodiments, an affinity tag is located at the C-terminus of a fusion protein sequence. In some embodiments, an affinity tag is located at the N-terminus of a fusion protein sequence. Non-limiting examples of affinity tags include the following tags: BP, FLAG, GST, HA, HBH, MBP, Myc, poly His, S-tag, SUMO, TAP, TRX, and V5.

In some embodiments, a fusion protein comprises a nuclear localization signal sequence. For example, a nuclear localization sequence may be located at the C-terminus and/or N-terminus of a protein sequence (e.g., a Cas protein or a ILF3 sequence). In some embodiments, a nuclear localization sequence is located between one or more domains of a protein sequence. In some embodiments, a nuclear localization signal sequence comprises an amino acid sequence at least 70% (e.g., at least 75%, 80%, 85%, 90%, 95%, 98%, 99%, or 100%) SEQ ID NO: 6 or 67. Any of the proteins provided herein may be produced by any method known in the art.

In some embodiments, a fusion protein comprises a GQSY-repeat motif corresponding to an ILF3 GQSY-repeat motif. Without wishing to be bound by any particular theory, the ILF3 sequence in a Cas-ILF3 fusion protein may not require one or more ILF3 RNA-binding domains to target the fusion protein to RNA because the Cas protein comprises one or more RNA-binding domains, e.g., one or more crRNA binding domains. In some embodiments, the fusion protein further comprises one or more of the following domains: a double-stranded RNA-binding domain 1 (dsRBD1) domain, a double-stranded RNA-binding domain 2 (dsRBD2) domain, a nuclear localization signal (NLS), a RGG-repeat motif, NES domain, a DZF domain, a NVKQ motif (NVKQ), and one or more Cas protein domains. In some embodiments, the fusion protein comprises the same domains corresponding to a wild-type NF90 ILF3. In some embodiments, the one or more Cas protein domains corresponding to one or more of the following domains that correspond to Cas13 domains: Helical-1, Lid, and/or Helical-2 domains. Any of the ILF3 domains or motifs may be mutated relative to a wild-type ILF3 domain or motif. In some embodiments, the domains of an ILF3 sequence are arranged in the order shown in FIG. 1A. In some embodiments, an ILF3 sequence lacks one or more of the domains show in FIG. 1A, but the remaining domains are arranged in the order shown in FIG. 1A. Any of the Cas domains or motifs may be mutated relative to a wild-type Cas domain or motif.

In some embodiments, a fusion protein comprises an amino acid sequence that is at least 70% (e.g., at least 75%, 80%, 85%, 90%, 95%, 98%, 99%, or 100%) identical to 1-14, 61-63, 66-69, and 80-81.

Aspects of the present disclosure provide nucleic acids encoding any of the fusion proteins and/or RNA-binding proteins disclosed herein. In some embodiments, an engineered nucleic acid comprises a promoter that is operably linked to a sequence encoding a fusion protein and/or RNA-binding protein. In some embodiments, an engineered nucleic acid is an expression vector.

### Trigger Nucleic Acids and Expression Vectors Capable of Inducing RNA Decay

Aspects of the present disclosure provide engineered nucleic acids that are shorter in length than an mRNA transcript of a gene of interest. In some embodiments, an engineered nucleic acid that is shorter in length than an mRNA transcript of a gene of interest is capable of inducing expression of the gene of interest and is referred to as a “trigger nucleic acid.” In some embodiments, an engineered nucleic acid is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, or 300 nucleotides in length.

In some embodiments, an engineered nucleic acid comprises 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, or 300 contiguous nucleotides of a trigger nucleic acid disclosed herein. In some embodiments, the trigger nucleic acid comprises the sequence: UGUUCGUGACAUAAAGGAGAAGCUGUGCUAUGUUGCCCUGGAUUUUGAGCAAGA AAUGGCUACUGCUGCAUCAUC (SEQ ID NO: 87). In some embodiments, the entire trigger nucleic acid is composed of ribonucleic acids.

In some embodiments, an engineered nucleic acid is complementary to a segment of a gene of interest. In some embodiments, an engineered nucleic acid is complementary to a segment of a paralog of a gene of interest. In some embodiments, an engineered nucleic acid is not complementary to a segment of a paralog of a gene of interest. In some embodiments, an engineered nucleic acid identified through a trigger screen using nucleic acid fragments of a gene of interest targets antisense RNA of a paralog of the gene of interest, which may be useful in identifying trigger nucleic acids for autosomal recessive disorders. In some embodiments, an engineered nucleic acid identified through a trigger screen using nucleic acid fragments of a gene of interest targets antisense RNA of the gene of interest (e.g., the gene where the mutation exists like ACTG1), which may be useful in identifying trigger nucleic acids for haploinsufficiency disorders. For example, a trigger nucleic acid can be used to upregulate the wild-type allele in case of a heterozygous mutation.

In some embodiments, an engineered nucleic acid disclosed herein is a ribonucleic acid and comprises a 5′ cap. Non-limiting examples of 5′ caps include 3′-O-Me-m⁷G(5′)ppp(5′)G; m⁷G(5′)ppp(5′)G; G(5′)ppp(5′)G; m⁷G(5′)ppp(5′)A; and G(5′)ppp(5′)A. In some embodiments, a 5′ cap is m⁷G(5′)ppp(5′)G. In some embodiments, the engineered nucleic acid is a trigger nucleic acid.

In some embodiments, an engineered nucleic acid disclosed herein comprises an internucleoside linkage modification and/or a modified nucleotide. A modified nucleotide may comprise a modified sugar moiety and/or a modified base moiety. In some instances, a modified sugar moiety comprises a 2′-OH group modification and/or a bridging moiety. 2′-OH group modifications include 2′-O-methyl (2′-O-Me), 2′-fluoro (2′-F), and 2′-O-methoxy-ethyl (2′-O-MOE or 2′-O-methoxyethyl (2′-MOE)). In some instances, a nucleotide with a bridging moiety is a locked nucleic acid. Non-limiting examples of modified bases include 2′-O-methoxyethyl base, deoxyuridine (dU), a 5-Methyl deoxyCytidine (5-methyl dC), and an inverted dT. Non-limiting examples of internucleoside linkage modifications include phosphorothioate (PS), boranophosphate, phosphoramidate, phosphorodiamidate morpholino (PMO), and thiophosphoramidate.

In some embodiments, an engineered nucleic acid disclosed herein further comprises a start codon at the 5′ end of the engineered nucleic acid. In some embodiments, the nucleic acid sequence comprises ATG or AUG at the 5′ end of the engineered nucleic acid. In some embodiments, an engineered nucleic acid disclosed herein further comprises a stop codon. In some embodiments, the stop codon is TAA or UAA. As a non-limiting example, a trigger nucleic acid may comprise: AUGUGUUCGUGACAUAAAGGAGAAGCUGUGCUAUGUUGCCCUGGAUUUUGAGCA AGAAAUGGCUACUGCUGCAUCAUCUAA (SEQ ID NO: 88), in which the start codon is underlined and the stop codon is underlined and italicized.

In some embodiments, an engineered nucleic acid disclosed herein comprises an ILF3 motif. In some embodiments, an ILF3 motif comprises one or more of the following:

(SEQ 89)
a) CATCCCT;
(SEQ 90)
b) ATCCCTG;
(SEQ 91)
c) CCCATCC;
(SEQ 92)
d) CACTTCC;
(SEQ 93)
e) TCCCTTC;
(SEQ 94)
f) TCCCATC;
(SEQ 95)
g) TCCCCTC;
(SEQ 96)
h) CCCTTCT;
(SEQ 97)
i) CCCTCTT;
and/or
(SEQ 98)
j) CCTACCC.

In some embodiments, an engineered nucleic acid is a ribonucleic acid and comprises any one of:

(SEQ 99)
a) CAUCCCU;
(SEQ 100)
b) AUCCCUG;
(SEQ 101)
c) CCCAUCC;
(SEQ 102)
d) CACUUCC;
(SEQ 103)
e) UCCCUUC;
(SEQ 104)
f) UCCCAUC;
(SEQ 105)
g) UCCCCUC;
(SEQ 106)
h) CCCUUCU;
(SEQ 107)
i) CCCUCUU;
and/or
(SEQ 108)
j) CCUACCC.

In some embodiments, an ILF3 motif may be present in a perturbed gene sequence.

In some embodiments, an engineered nucleic acid is an engineered ribonucleic acid. In some embodiments, the engineered ribonucleic acid is a trigger ribonucleic acid. In some embodiments, an engineered nucleic acid is less than 100 nucleotides in length (e.g., less than 99 nucleotides, less than 98 nucleotides, less than 97 nucleotides, less than 96 nucleotides, less than 95 nucleotides, less than 94 nucleotides, less than 93 nucleotides, less than 92 nucleotides, less than 91 nucleotides, less than 90 nucleotides, less than 89 nucleotides, less than 88 nucleotides, less than 87 nucleotides, less than 86 nucleotides, less than 85 nucleotides, less than 84 nucleotides, less than 83 nucleotides, less than 82 nucleotides, less than 81 nucleotides, less than 80 nucleotides, less than 79 nucleotides, less than 78 nucleotides, less than 77 nucleotides, less than 76 nucleotides, less than 75 nucleotides, less than 74 nucleotides, less than 73 nucleotides, less than 72 nucleotides, less than 71 nucleotides, less than 70 nucleotides, less than 69 nucleotides, less than 68 nucleotides, less than 67 nucleotides, less than 66 nucleotides, less than 65 nucleotides, less than 64 nucleotides, less than 63 nucleotides, less than 62 nucleotides, less than 61 nucleotides, less than 60 nucleotides, less than 59 nucleotides, less than 58 nucleotides, less than 57 nucleotides, less than 56 nucleotides, less than 55 nucleotides, less than 54 nucleotides, less than 53 nucleotides, less than 52 nucleotides, less than 51 nucleotides, less than 50 nucleotides, less than 49 nucleotides, less than 48 nucleotides, less than 47 nucleotides, less than 46 nucleotides, less than 45 nucleotides, less than 44 nucleotides, less than 43 nucleotides, less than 42 nucleotides, less than 41 nucleotides, less than 40 nucleotides, less than 39 nucleotides, less than 38 nucleotides, less than 37 nucleotides, less than 36 nucleotides, less than 35 nucleotides, less than 34 nucleotides, less than 33 nucleotides, less than 32 nucleotides, less than 31 nucleotides, less than 30 nucleotides, less than 29 nucleotides, less than 28 nucleotides, less than 27 nucleotides, less than 26 nucleotides, less than 25 nucleotides, less than 24 nucleotides, less than 23 nucleotides, less than 22 nucleotides, less than 21 nucleotides, less than 20 nucleotides, less than 19 nucleotides, less than 18 nucleotides, less than 17 nucleotides, less than 16 nucleotides, or less than 15 nucleotides in length). In some embodiments an engineered nucleic acid is between 22 and 31 nucleotides in length.

In some embodiments, an engineered nucleic acid comprises a sequence that is at least 70% (e.g., at least 75%, 80%, 85%, 90%, 95%, 98%, 99%, or 100%) identical to any one of SEQ ID NOs: 36-60. In some embodiments, an engineered nucleic acid is a trigger ribonucleic acid and comprises a sequence that is at least 70% (e.g., at least 75%, 80%, 85%, 90%, 95%, 98%, 99%, or 100%) identical to any one of SEQ ID NOs: 37-40 and 87-88. See, e.g., Table 3 and Example 2.

Without wishing to be bound by any particular theory, targeting nascent antisense RNAs with a trigger ribonucleic acid at the locus of the gene it is upregulating may allow for tissue specific upregulation as antisense RNAs are tissue-specific. Furthermore, in some embodiments, the disclosure herein demonstrates that in some embodiments, a trigger ribonucleic acid identified by a method disclosed herein induces a 2-fold upregulation of expression levels which is comparable to physiological upregulation level. Without wishing to be bound by any particular theory, such trigger ribonucleic acids may be advantageous over other methods and systems of increasing gene expression, such as mRNA therapy, that may lead to very high upregulation levels (100-1000×) that may not be suitable for certain genetic diseases (as haploinsufficiency disorders). The trigger RNAs can be used therapeutically for genetic diseases to increase the expression levels of a paralog or the wild-type unaffected allele (in case of haploinsufficiency disorders). In some embodiments the trigger RNAs can be used therapeutically for genetic diseases to increase the expression levels of a mutant protein that retains some functional activity. In some embodiments, the trigger RNAs can also be a platform to design RNAs that can promote gene expression.

In some embodiments, a trigger nucleic acid increases expression of a gene of interest by 1.1-fold, 1.2-fold, 1.3-fold, 1.4-fold, 1.5-fold, 1.6-fold, 1.7-fold, 1.8-fold, 1.9-fold, 2-fold, 2.1-fold, 2.2-fold, 2.3-fold, 2.4-fold, 2.5-fold, 2.6-fold, 2.7-fold, 2.8-fold, 2.9-fold, 3-fold, 3.1-fold, 3.2-fold, 3.3-fold, 3.4-fold, 3.5-fold, 3.6-fold, 3.7-fold, 3.8-fold, 3.9-fold, 4-fold, 4.1-fold, 4.2-fold, 4.3-fold, 4.4-fold, 4.5-fold, 4.6-fold, 4.7-fold, 4.8-fold, 4.9-fold, or 5-fold.

In some embodiments, a trigger nucleic acid increases expression of a gene of interest by 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, or 500%.

In some embodiments, an engineered nucleic acid is an antisense oligonucleotide that comprises deoxyribonucleic acids. In some embodiments, an antisense oligonucleotide comprises one or more modifications. In some embodiments, an antisense oligonucleotide comprises a phosphorothioate linkage, a 2′-O-methoxyethyl base, and/or a locked nucleic acid.

In some embodiments, an engineered nucleic acid comprises an engineered nucleic acid sequence that is at least 70% (e.g., at least 75%, 80%, 85%, 90%, 95%, 98%, 99%, or 100%) identical to any one of SEQ ID NOS: 48-60. In some embodiments, an engineered nucleic acid is a trigger deoxyribonucleic acid and comprises a sequence that is at least 70% (e.g., at least 75%, 80%, 85%, 90%, 95%, 98%, 99%, or 100%) identical to any one of SEQ ID NOs: 49-55 or 58-60. In some embodiments, a trigger nucleic acid is an antisense oligonucleotide. Methods and systems disclosed herein may be used to identify regions of a transcript to be targeted to increase gene expression. In some embodiments, screening of candidate trigger nucleic acids informs design of antisense oligonucleotides (e.g., ASOs design guided by trigger screens (ASOdgT)). In some embodiments, an engineered nucleic acid is a trigger deoxyribonucleic acid and comprises a sequence that is at least 70% (e.g., at least 75%, 80%, 85%, 90%, 95%, 98%, 99%, or 100%) identical to any one of the antisense oligonucleotides targeting regions identified from trigger screens.

In some embodiments, a trigger nucleic acid is encoded by an expression vector that is capable of inducing RNA decay. In some embodiments, a trigger nucleic acid is encoded by an expression vector capable of inducing nonsense-mediated decay, no-go decay, or no-stop decay, including any of the expression vectors disclosed herein.

In some embodiments, a trigger nucleic acid is identified using a method or system disclosed herein.

Compositions Comprising an RNA-Binding Protein and/or an Engineered Nucleic Acid Disclosed Herein

Any of the nucleic acids disclosed herein, including any of the nucleic acids encoding a RNA-binding protein (e.g., Cas protein, ILF3 protein sequence, and/or Cas-ILF3 fusion protein), a trigger nucleic acid, an oligonucleotide, and/or a guide RNA disclosed herein may be delivered to a cell, tissue, organ, or subject as a nucleic acid, e.g., by means of transfection, or electroporation, or can be conjugated to molecules for promoting uptake by target cells. In some embodiments, a nucleic acid is an expression vector, which may include expression control sequences, including promoters, enhancers, transcription signal sequences, transcription termination sequences, polyadenylation signals, Kozak consensus sequences, introns, and/or internal ribosome entry sites (IRES). In some embodiments, a vector may also comprise a sequence encoding a nuclear localization and/or a sequence encoding a nuclear export signal sequence linked to a sequence coding for a protein.

Non-limiting examples of vectors include plasmid vectors and viral vectors. In some embodiments, a viral vector is based on adenoviruses (Ads), retroviruses (7-retroviruses and lentiviruses), poxviruses, adeno-associated viruses, baculoviruses, and herpes simplex viruses. Viruses or virus-like particles (VLPs) may also be used to deliver any of the engineered nucleic acids disclosed herein. Viral vectors and viral particles may be engineered to incorporate targeting ligands for targeting particular tissues.

In some embodiments, an engineered virus is used to deliver a sequence of interest (e.g., a sequence encoding a RNA-binding protein, guide RNA, oligonucleotide and/or trigger nucleic acid disclosed herein) into a cell. In some embodiments, an engineered virus comprises (i) a heterologous nucleic acid region encoding a sequence of interest and (2) one or more nucleotide sequences comprising a sequence that facilitates expression of the heterologous nucleic acid region (e.g., a promoter), and (3) one or more nucleic acid regions comprising a sequence that facilitate integration of the heterologous nucleic acid region (optionally with the one or more nucleic acid regions comprising a sequence that facilitates expression) into the genome of a cell. In some embodiments, viral sequences that facilitate integration comprise Inverted Terminal Repeat (ITR) sequences. In some embodiments, a nucleotide sequence encoding a sequence of interest is flanked on each side by an ITR sequence. In some embodiments, the nucleic acid vector further comprises a region encoding an AAV Rep protein, either contained within the region flanked by ITRs or outside the region. The ITR sequences can be derived from any AAV serotype (e.g., 1, 2, 3, 4, 5, 6, 7, 8, 9, or 10) or can be derived from more than one serotype.

ITR sequences and plasmids containing ITR sequences are known in the art and commercially available (see, e.g., products and services available from Vector Biolabs, Philadelphia, PA; Cellbiolabs, San Diego, CA; Agilent Technologies, Santa Clara, Ca; and Addgene, Cambridge, MA; and Gene delivery to skeletal muscle results in sustained expression and systemic delivery of a therapeutic protein. Kessler P D, Podsakoff G M, Chen X, McQuiston S A, Colosi P C, Matelis L A, Kurtzman G J, Byrne B J. Proc Natl Acad Sci USA. 1996 Nov. 26; 93(24):14082-7; and Curtis A. Machida. Methods in Molecular Medicine™. Viral Vectors for Gene Therapy Methods and Protocols. 10.1385/1-59259-304-6:201 © Humana Press Inc. 2003. Chapter 10. Targeted Integration by Adeno-Associated Virus. Matthew D. Weitzman, Samuel M. Young Jr., Toni Cathomen and Richard Jude Samulski; U.S. Pat. Nos. 5,139,941 and 5,962,313, all of which are incorporated herein by reference).

Any of the engineered ribonucleic acids disclosed herein, including any of the trigger ribonucleic acids disclosed herein, may be incorporated into a ribonucleoprotein complex with an ILF3 sequence. In some embodiments, the engineered ribonucleic acid is less than 300 nucleotides in length. In some embodiments, the engineered ribonucleic acid is between 22 and 31 nucleotides in length. In some embodiments, the engineered ribonucleic acid is at least 70% (e.g., at least 75%, 80%, 85%, 90%, 95%, 98%, 99%, or 100%) identical to any one of SEQ ID NOs: 37-40 and 87-88. In some embodiments, the engineered ribonucleic acid is at least 70% (e.g., at least 75%, 80%, 85%, 90%, 95%, 98%, 99%, or 100%) identical to any one of the oligonucleotides identified in a method disclosed herein as being capable of increasing expression of a gene of interest.

In some embodiments, a ribonucleoprotein complex is formed between a protein comprising a Cas protein and a guide RNA. In some embodiments, the ribonucleoprotein complex comprises (i) a fusion protein with a Cas protein and an ILF3 sequence and (ii) a guide RNA. In some embodiments, a guide RNA comprises a sequence that is complementary to an antisense transcript (e.g., complementary to a portion of an antisense transcript). In some embodiments, a guide RNA comprises a sequence that is complementary to a sense transcript (e.g., complementary to a portion of a sense transcript). In some embodiments, a guide RNA comprises a sequence that is at least 70% (e.g., at least 75%, 80%, 85%, 90%, 95%, 98%, 99%, or 100%) identical to any one of SEQ ID NOs: 15-34. In some embodiments, a guide RNA is complementary to an intron sequence of a gene. In some embodiments, a guide RNA is complementary to an exon sequence of a gene.

Any of the engineered nucleic acids and/or proteins disclosed herein may be delivered via a lipid nanoparticle. The term “lipid nanoparticle” or “LNP” refers to spherical vesicle made at least in part of ionizable lipids. The diameter of lipid nanoparticle varies and ranges between 10 and 1000 nanometers. The core of a lipid nanoparticle comprises a matrix of solubilized lipid molecules and is stabilized by surfactants. The compositions of lipid nanoparticles vary depending on the therapeutic purpose. Examples of components, formulations, and applications of lipid nanoparticles may be found in Hou et al. Lipid nanoparticles for mRNA delivery. Nature Rev Mat. 6:1078-1094 (2021).

In some embodiments, a LNP comprises cationic, anionic, and/or neutral lipids. In

some embodiments, an LNP may comprise neutral lipids, such as the fusogenic phospholipid1,2-Dioleoyl-sn-glycero-3-phosphoethanolamine (DOPE) or the membrane component cholesterol, which may be used as lipids that help enhance transfection activity and nanoparticle stability. In some embodiments, an LNP may comprise hydrophobic lipids, hydrophilic lipids, or both.

In some embodiments, the engineered nucleic acids and/or proteins disclosed herein are delivered via electroporation to a cell. In some embodiments, the engineered nucleic acids and/or proteins are delivered to a cell in a subject via a lipid nanoparticle, recombinant virus, and/or viral vector.

### Host Cells and Pharmaceutical Compositions

Any of the proteins and nucleic acids disclosed herein may be delivered to a cell, tissue, organ, and/or subject in compositions according to any appropriate method known in the art. In some embodiments, the cell is a eukaryotic cell. In some embodiments, the cell is a mammalian cell. In some embodiments, the cell is a human cell. In some embodiments, the cell is a mouse cell. In some embodiments, the cell is from a non-human mammal. In some embodiments, the cell is from a domesticated animal. In some embodiments, the cell is from a research animal. In some embodiments, the cell is a plant cell.

The protein and/or nucleic acid, preferably suspended in a physiologically compatible carrier (i.e., in a composition), may be administered to a subject, e.g., host animal, patient, experimental animal. In some embodiments, the subject is a mammal. In some examples, the mammal is a human. In other embodiments, the mammal can be a non-human mammal, such as a human, mouse, rat, cat, dog, sheep, rabbit, horse, cow, goat, pig, guinea pig, hamster, chicken, turkey, or a non-human primate (e.g., cynomolgus monkey). The subject may be at any stage of development and of any gender. In some embodiments, a composition disclosed herein is administered to a plant.

The protein and/or nucleic acid can be delivered to any organ or tissue of interest. One of ordinary skill in the art would be able to select proteins and/or nucleic acids according to the specific tissue being targeted.

The compositions of the disclosure may comprise an engineered nucleic acid described herein alone, or in combination with one or more other engineered nucleic acids (e.g., two or more trigger nucleic acids). In some embodiments, a composition comprises 1, 2, 3, 4, 5, 6, 7, 8, 9, 10, or more different trigger nucleic acids.

In some embodiments, a composition further comprises a pharmaceutically acceptable carrier. Suitable carriers may be readily selected by one of skill in the art in view of the indication for which the protein and/or nucleic acid is directed. “Acceptable” means that the carrier must be compatible with the protein and/or the nucleic acid of the composition (and preferably, capable of stabilizing the active ingredient) and not deleterious to the subject to be treated. In some embodiments, the pharmaceutically acceptable carrier/excipient is compatible with the mode of administration. Pharmaceutically acceptable excipients (carriers) including buffers, which are well known in the art. See, e.g., Remington: The Science and Practice of Pharmacy 20th Ed. (2000) Lippincott Williams and Wilkins, Ed. K. E. Hoover. For example, one acceptable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present disclosure.

The protein and/or nucleic acid containing pharmaceutical composition disclosed herein may further comprise a suitable buffer agent. A buffer agent is a weak acid or base used to maintain the pH of a solution near a chosen value after the addition of another acid or base. In some examples, the buffer agent disclosed herein can be a buffer agent capable of maintaining physiological pH despite changes in carbon dioxide concentration (e.g., produced by cellular respiration). Exemplary buffer agents include, but are not limited to, HEPES (4-(2-hydroxyethyl)-1-piperazineethanesulfonic acid) buffer, Dulbecco's phosphate-buffered saline (DPBS) buffer, or phosphate-buffered saline (PBS) buffer. Such buffers may comprise disodium hydrogen phosphate and sodium chloride, or potassium dihydrogen phosphate and potassium chloride.

Optionally, the compositions of the disclosure may contain, in addition to the protein and/or nucleic acid and carrier(s), other pharmaceutical ingredients, such as preservatives or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The protein and/or nucleic acid containing pharmaceutical composition described herein may comprise one or more suitable surface-active agents, such as a surfactant. Surfactants are compounds that lower the surface tension (or interfacial tension) between two liquids, between a gas and a liquid, or between a liquid and a solid. Surfactants may act as detergents, wetting agents, emulsifiers, foaming agents, and dispersants. Suitable surfactants include, in particular, non-ionic agents, such as polyoxyethylenesorbitans (e.g., Tween™ 20, 40, 60, 80 or 85) and other sorbitans (e.g., Span™ 20, 40, 60, 80 or 85). Compositions with a surface active agent will conveniently comprise between 0.05 and 5% surface-active agent, and can be between 0.1 and 2.5%. It will be appreciated that other ingredients may be added, for example, mannitol or other pharmaceutically acceptable vehicles, if necessary.

In some embodiments, the proteins and/or nucleic acids are administered in sufficient amounts to transfect the cells of a desired tissue and to provide sufficient levels of gene transfer and/or upregulate gene expression without undue adverse effects. Examples of pharmaceutically acceptable routes of administration include, but are not limited to, direct delivery to the selected organ or tissue, intravenous, intramuscular, subcutaneous, intradermal, intratumoral, and other parental routes of administration. Routes of administration may be combined, if desired.

In some embodiments, a dose of protein and/or nucleic acid is administered to a subject no more than once per day (e.g., a 24-hour period). In some embodiments, a dose of protein and/or nucleic acid is administered to a subject no more than once per 2, 3, 4, 5, 6, or 7 days. In some embodiments, a dose of protein and/or nucleic acid is administered to a subject no more than once per week (e.g., 7 calendar days). In some embodiments, a dose of protein and/or nucleic acid is administered to a subject no more than bi-weekly (e.g., once in a two-week period). In some embodiments, a dose of protein and/or nucleic acid is administered to a subject no more than once per month (e.g., once in 30 calendar days). In some embodiments, a dose of protein and/or nucleic acid is administered to a subject no more than once per six months. In some embodiments, a dose of protein and/or nucleic acid is administered to a subject no more than once per year (e.g., 365 days or 366 days in a leap year). In some embodiments, a dose of protein and/or nucleic acid is administered to a subject once in a lifetime.

Formulation of pharmaceutically acceptable excipients and carrier solutions is well known to those of skill in the art, as is the development of suitable dosing and treatment regimens for using the particular compositions described herein in a variety of treatment regimens. Factors, such as solubility, bioavailability, biological half-life, route of administration, product shelf life, as well as other pharmacological considerations, will be contemplated by one skilled in the art of preparing such pharmaceutical formulations, and as such, a variety of dosages and treatment regimens may be desirable.

In some embodiments, proteins and/or nucleic acids in suitably formulated pharmaceutical compositions disclosed herein are delivered directly to target tissue. However, in certain circumstances it may be desirable to separately or in addition deliver the protein- and/or nucleic acid-based therapeutic constructs via another route, e.g., subcutaneously, parenterally, intravenously, intramuscularly, intrathecally, orally, or intraperitoneally. In some embodiments, the administration modalities as described in U.S. Pat. Nos. 5,543,158; 5,641,515 and 5,399,363 (each specifically incorporated herein by reference in its entirety) may be used to deliver an engineered nucleic acid.

The pharmaceutical forms suitable for injectable use include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases, the form is sterile. It must be stable under the conditions of manufacture and storage and must be preserved to prevent contamination with microorganisms, such as bacteria, fungi, and other viruses. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of contamination by microorganisms can be achieved by various antibacterial and antifungal agents, for example, parabens, chlorobutanol, phenol, sorbic acid, thimerosal, and the like. In many cases, it will be preferable to include isotonic agents, for example, sugars or salts (e.g., sodium chloride). Prolonged absorption of the injectable composition can be achieved by the use in the composition of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous administration, intramuscular administration, subcutaneous administration, or intraperitoneal administration. In this respect, a suitable sterile aqueous medium may be employed. For example, one dosage may be dissolved in 1 ml of isotonic NaCl solution and either added to 1000 ml of hypodermoclysis fluid or injected at the proposed site of infusion (see for example, Remington's Pharmaceutical Sciences 15th Edition, pages 1035-1038 and 1570-1580). Some variation in dosage will necessarily occur depending on the condition of the host. The person responsible for administration will, in any event, determine the appropriate dose for the individual subject/host.

Sterile injectable solutions are prepared by incorporating the active protein and/or nucleic acid in the required amount in the appropriate solvent with various of the other ingredients described herein, as required, followed by filter sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The nucleic acid and protein compositions disclosed herein may also be formulated in a neutral or salt form. Pharmaceutically acceptable salts include but are not limited to hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, solvents, coatings, diluents, antibacterial and antifungal agents, isotonic and absorption delaying agents, buffers, carrier solutions, suspensions, colloids, and the like. The use of such media and agents for pharmaceutically active substances is well known in the art. Supplemental active ingredients can also be incorporated into the compositions. The phrase “pharmaceutically acceptable” refers to molecular entities and compositions that do not produce an allergic or similar untoward reaction when administered to a host.

Delivery vehicles such as liposomes, nanocapsules, microparticles, microspheres, lipid particles, vesicles, and the like, may be used for the introduction of the compositions of the present disclosure into suitable host cells. In particular, the proteins and/or nucleic acids may be formulated for delivery either encapsulated in a lipid particle, a liposome, a vesicle, a nanosphere, a nanoparticle, or the like.

Such formulations may be preferred for the introduction of pharmaceutically acceptable formulations of the nucleic acids or the protein constructs disclosed herein. The formation and use of liposomes are generally known to those of skill in the art. Recently, liposomes were developed with improved serum stability and circulation half-times (U.S. Pat. No. 5,741,516, which is incorporated herein by reference). Further, various methods of liposome and liposome-like preparations as potential drug carriers have been described (U.S. Pat. Nos. 5,567,434; 5,552,157; 5,565,213; 5,738,868 and 5,795,587, each of which is incorporated herein by reference).

Alternatively, nanocapsule formulations of the protein and/or nucleic may be used. Nanocapsules can generally entrap substances in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use.

### Kits and Related Compositions

The agents described herein may, in some embodiments, be assembled into pharmaceutical or research kits to facilitate their use in therapeutic, or research applications. A kit may include one or more containers housing the components (e.g., nucleic acids, protein and/or nucleic acid) of the disclosure and instructions for use. Specifically, such kits may include one or more agents described herein, along with instructions describing the intended application and the proper use of these agents. In certain embodiments, agents in a kit may be in a pharmaceutical formulation and dosage suitable for a particular application and for a method of administration of the agents. Kits for research purposes may contain the components in appropriate concentrations or quantities for performing various experiments.

In some embodiments, the instant disclosure relates to a kit for administering a protein and/or nucleic acid as described herein. In some embodiments, the kit comprising a container housing the protein and/or nucleic acid, and devices (e.g., syringe) for extracting the protein and/or nucleic acid from the housing. In some embodiments, the device for extracting the protein and/or nucleic acid from the housing is also used for administration (e.g., injection).

In some embodiments, the instant disclosure relates to a kit for a disease associated with the gene product. In some embodiments, the kit is for delivering a functional gene product to a target cell using gene therapy (e.g., protein and/or nucleic acid described herein).

The kit may be designed to facilitate use of the methods described herein by researchers and can take many different forms. Each of the compositions of the kit, where applicable, may be provided in liquid form (e.g., in solution), or in solid form (e.g., a dry powder). In certain cases, some of the compositions may be constitutable or otherwise processable (e.g., to an active form), for example, by the addition of a suitable solvent or other medium (for example, water or a cell culture medium), which may or may not be provided in the kit. As used herein, “instructions” can include a component of instruction and/or promotion, and typically involve written instructions on or associated with the packaging. Instructions also can include any oral or electronic instructions provided in any manner such that a user will clearly recognize that the instructions are to be associated with the kit, for example, audiovisual (e.g., videotape, DVD, CD-ROM, website links for downloadable file, etc.), Internet, and/or web-based communications, etc. The written instructions may be in a form prescribed by a governmental agency regulating the manufacture, use, or sale of pharmaceuticals or biological products, which instructions can also reflect approval by the agency of manufacture, use, or sale for animal administration.

The kit may contain any one or more of the components described herein in one or more containers. As an example, in one embodiment, the kit may include instructions for mixing one or more components of the kit and/or isolating and mixing a sample and applying to a subject. The kit may include a container housing the protein and/or nucleic acid described herein. The protein and/or nucleic acid may be in the form of a liquid, gel, or solid (powder). The protein and/or nucleic acid may be prepared sterilely, packaged in a syringe, and shipped refrigerated. Alternatively, the protein and/or nucleic acid may be housed in a vial or other container for storage. A second container may have other agents prepared sterilely.

Alternatively, the kit may include the protein and/or nucleic acid premixed and shipped in a syringe, vial, tube, or other container.

### Methods of Identifying Engineered Nucleic Acids to Increase Gene Expression

Aspects of the present disclosure provide methods, compositions, and systems for identifying one or more oligonucleotides that are shorter than an mRNA encoding a gene of interest in which the one or more oligonucleotides are capable of upregulating expression of the gene of interest.

In some embodiments, the method comprises using an expression vector that is capable of inducing RNA decay. See, e.g., the expression vectors disclosed herein. In some embodiments, the method comprises contacting eukaryotic cells with a population of expression vectors in which each expression vector (i) is capable of inducing RNA decay and (ii) encodes an oligonucleotide. In some embodiments, an oligonucleotide is 10-300 nucleotides in length. In some embodiments, an oligonucleotide is 15-120 nucleotides long and comprises a sequence of at least 10 contiguous nucleotides that is complementary to a target sequence. In some embodiments, the oligonucleotide is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, or 300 nucleotides in length. In some embodiments, an oligonucleotide encodes a sequence selected from SEQ ID NOs: 36-60 or a fragment thereof. In some embodiments, an oligonucleotide encodes a sequence that is at least 70% identical to a sequence selected from any one of SEQ ID NOs: 36-60.

Further aspects of the present disclosure provide a plurality of expression vectors in which each expression vector (i) is capable of inducing RNA decay and (ii) encodes an oligonucleotide. Several surveillance pathways are used by cells to maintain the fidelity of mRNA. These pathways generally mark aberrant mRNA for degradation. Any expression vector that is capable of inducing mRNA may be used to identify nucleic acids that are capable of increasing gene expression (to identify “triggers”). For example, an expression vector that is capable of inducing mRNA may be used to identify ribonucleic acid fragments that are capable of inducing transcriptional adaptation. Such methods of identifying nucleic acids that are capable of increasing gene expression using RNA decay expression vectors may be referred to as trigger screens.

In some embodiments, the eukaryotic cell is a mouse cell. In some embodiments, the eukaryotic cell is a human cell. In some embodiments, the eukaryotic cell comprises a nucleic acid sequence encoding an ILF3 sequence. In some embodiments, the eukaryotic cell comprises a nucleic acid encoding a fusion protein comprising a Cas protein and a ILF3 sequence.

Nonsense-mediated decay is a surveillance pathway used by cells to eliminate and/or degrade mRNA transcripts that comprise one or more premature stop codons (PTC). See, e.g., Kurosaki et al., Nat Rev Mol Cell Biol. 2019 July; 20(7):406-420. An expression vector that is capable of inducing nonsense-mediated generally comprises one or more premature stop codons following an oligonucleotide sequence of interest. In some embodiments, an expression vector that induces nonsense-mediated decay comprises: a first stop codon following an oligonucleotide sequence of interest; an intron of a second gene linked to an exon of the second gene; and a second stop codon following the exon of the second gene. In some embodiments, the second gene is the Hemoglobin Subunit Beta (HBB) gene. In some embodiments, an expression vector that induces nonsense-mediated decay comprises a plurality of sets of introns and exons of a second gene and each set of introns and exons is followed by a stop codon. In some embodiments, an expression vector comprises 2, 3, 4, 5, 6, 7, 8, 9, 10, 15, 20, 30, 40, 50, 60, 70, 80, 90, 100, 200, 300, 400, or 500 sets of introns and exons.

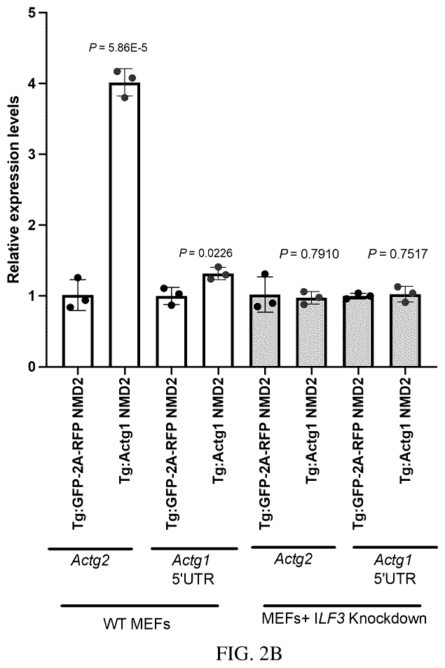

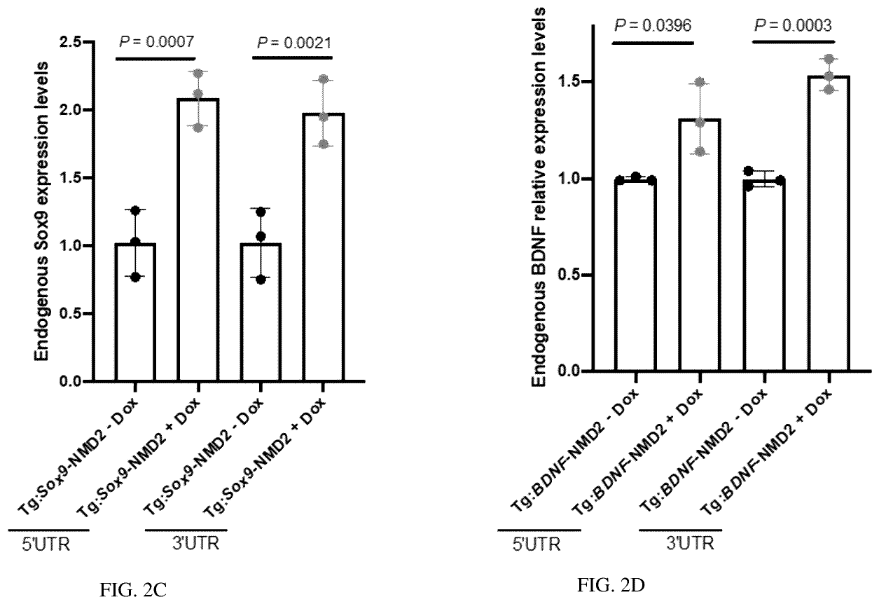

In some embodiments, an expression vector that is capable of inducing nonsense-mediated decay comprises a promoter operably linked to nucleic acid segments in the following order: (i) a nucleic acid sequence encoding an oligonucleotide followed by a stop codon; (ii) an exon of a second gene; (iii) an intron of the second gene; (iv) a second exon of the second gene; and (v) a second stop codon. In some embodiments, an expression vector that is capable of inducing nonsense-mediated decay comprises a promoter operably linked to nucleic acid segments encoding the following in sequential order: (i) an oligonucleotide followed by a stop codon; (ii) a first exon of a second gene; (iii) a first intron of the second gene; (v) a second exon of the second gene; (v) a second stop codon; (v) a third exon of the second gene; (vi) a second intron of the second gene; (vii) a fourth exon of the second gene; and (viii) a third stop codon. See also, e.g., FIG. 2A. In some embodiments, an expression vector that is capable of inducing nonsense-mediated decay is the top vector shown in FIG. 2A. In some embodiments, the first and third exons of the second gene are the same exon of the second gene. In some embodiments, the terms “first,” “second,” “third,” and “fourth” when used in reference to an intron or exon of a second gene does not specify the location of the intron or exon in the naturally occurring gene. For example, a first intron of the second gene may be the second intron of the naturally occurring version of the second gene. In some embodiments, the second and fourth exons of the second gene are the same exon of the second gene. In some embodiments, the first and second introns of the second gene are the same intron of the second gene. In some embodiments, the oligonucleotide encodes a segment of the gene of interest. In some embodiments, the oligonucleotide does not encode a segment of the gene of interest but encodes a segment of a paralog of the gene of interest. In some embodiments, the oligonucleotide encodes a segment of the gene of interest and a segment of a paralog of the gene of interest. In some embodiments, the segment of the gene of interest is the same as the segment of the paralog of the gene of interest. In some embodiments, the oligonucleotide encodes a trigger nucleic acid.

The No-Go Decay (NGD) mRNA surveillance pathway degrades mRNAs that have stalled ribosomes. Ribosomes may be stalled by a secondary structure that forms in the RNA. For example, an mRNA transcript may have sequences that are complementary to one another such that the complementary sequences hybridize to form a secondary structure. See, e.g., Doma et al., Nature 440, 561-564 (2006) and Pasos et al., Mol. Biol. Cell 20, 3025-3032 (2009). An expression vector that induces no-go decay may encode a promoter operably linked to an oligonucleotide sequence of interest and the expression vector may further encode a self-complementary sequence downstream of an oligonucleotide sequence of interest. Non-limiting examples of self-complementary sequences include single-stranded nucleic acids comprising one or more regions of complementarity to one or more other regions of the same single-stranded nucleic acid. When transcribed, such self-complementary sequences can form a secondary structure. In some embodiments, a self-complementary sequence forms a hairpin structure.

The non-stop decay or no-stop decay pathway detects and degrades mRNA transcripts that lack a proper stop codon. Such aberrant transcripts are detected during translation when the ribosome translates into the poly-lysine tails (including polyA tails) and stalls. See, e.g., Wiley Interdiscip Rev RNA. 2010 July-August; 1(1):132-41 and Navickas et al., Nat. Commun. 2020 Jan. 8; 11(1):122. In some embodiments, an expression vector capable of inducing non-stop decay comprises an expression vector that encodes an oligonucleotide sequence of interest and further encodes two or more contiguous lysine residues downstream of an oligonucleotide sequence of interest and does not include a stop codon between the oligonucleotide sequence of interest and the sequence encoding the two or more contiguous lysine residues. In some embodiments, the two or more contiguous lysine residues are encoded by a nucleic acid sequence comprising the sequence AAA and/or AAG. In some embodiments, the two or more contiguous lysine residues are encoded by a poly(A) sequence. In some instances, a poly(A) tail is 2, 3, 4, 5, 6, 7, 8, 9, 10, 15, 20, 30, 40, 50, 60, 70, 80, 90, 100, 200, 300, 400, or 500 nucleotides in length, including any values in-between.

Following contacting cells with the population of expression vectors that are capable of inducing RNA decay, the method of identifying a subset of the cells as having increased expression of the gene of interest as compared to control cells may further comprise identifying a subset of the cells as having increased expression of the gene of interest as compared to control cells; and detecting one or more oligonucleotides in the subset of the cells, thereby identifying one or more oligonucleotides that are capable of upregulating expression of a gene of interest. In some embodiments, control cells are cells that do not comprise an expression vector encoding one or more of the oligonucleotides. In some embodiments, control cells are cells that comprise a expression vector capable of inducing RNA decay but the expression vector does not encode one or more of the oligonucleotides.

Any suitable method to detect gene expression may be used to identify a subset of cells as having increased expression of a gene of interest relative to a control population, including FISH-Flow, which is a flow-cytometry-based method for measuring intracellular mRNA in cells using fluorescence in situ hybridization (FISH) probes, qPCR, and RNA-seq. See, e.g., Arrigucci et al., Nat. Protoc. 2017 June; 12(6):1245-1260.

In some embodiments, a method of identifying one or more oligonucleotides capable of upregulating gene expression comprises isolating ILF3 from a eukaryotic cell and detecting one or more ribonucleic acids bound to ILF3, thereby identifying the one or more oligonucleotides. In some embodiments, isolating ILF3 comprises tagging the ILF3 protein and using a non-antibody affinity reagent that binds to the tag to isolate ILF3. Generally, the isolation of ILF3 is performed under conditions suitable to maintain physical association of ILF3 with RNA that is bound to it in a cell.

In some embodiments, a method of identifying one or more oligonucleotides capable of upregulating gene expression comprises immunoprecipitating ILF3 from a eukaryotic cell and detecting one or more ribonucleic acids bound to ILF3, thereby identifying the one or more oligonucleotides. Generally, the immunoprecipitating of ILF3 is performed under conditions suitable to maintain physical association of ILF3 with RNA that is bound to it in a cell. The sequence of the one or more ribonucleic acids bound to ILF3 or a fragment of the one or more ribonucleic acids bound to ILF3 can be used to produce an oligonucleotide capable of upregulating gene expression. ILF3 may be immunoprecipitated using an antibody that binds a portion of ILF3. Non-limiting examples of ILF3 antibodies include ab92355 (ABCAM®) and BDB612155 (BD® Biosciences).

In some embodiments, the eukaryotic cell comprises a nonsense-mediated decay vector (NMD) vector an mRNA of interest or a homolog thereof and the method comprises identifying fragments of the mRNA of interest or homolog thereof that are bound to ILF3. In some embodiments, wherein the cell has been transfected with an oligonucleotide comprising a segment of a mRNA of interest or a homolog thereof. For example, one could transfect cells with multiple oligonucleotides comprising different portions of a mRNA of interest or a homolog thereof and then identify those that bind to ILF3.

Any suitable method may be used to detect one or more oligonucleotides of interest, including any suitable sequencing method.

In some embodiments, an oligonucleotide used in a method disclosed herein is a segment of a gene that is a paralog of a gene of interest. As a non-limiting example, expression vectors capable of inducing RNA decay and comprising an oligonucleotide that encodes a segment of a paralog of a gene of interest may be used in a method disclosed herein to identify the minimal sequence of the paralog that is sufficient to increase expression of the gene of interest. In some embodiments, an oligonucleotide that encodes a segment of a paralog gene is complementary to a segment of the gene of interest. In some embodiments, an oligonucleotide that encodes a segment of a paralog gene is identical to a segment of the gene of interest. In some embodiments, an oligonucleotide that encodes a segment of a segment of the gene of interest.

In some embodiments, an oligonucleotide disclosed herein is complementary to an antisense transcript. In some embodiments, an oligonucleotide disclosed herein is complementary to a sense transcript.

The sequence of an oligonucleotide identified by a method disclosed herein may be used to design a ribonucleic acid that is capable of inducing expression of a gene of interest (e.g., a trigger RNA). In some embodiments, a trigger RNA is encoded by a sequence that comprises an oligonucleotide identified by a method disclosed herein. In some embodiments, a trigger RNA is encoded by a sequence that is an oligonucleotide identified by a method disclosed herein. In some embodiments, a trigger RNA is complementary to an antisense transcript (e.g., to a portion of an antisense transcript).

In some embodiments, the sequence of an oligonucleotide identified by a method disclosed herein is incorporated into a deoxyribonucleic acid to produce an antisense oligonucleotide. In some embodiments, the antisense oligonucleotide is complementary to an antisense transcript.

### Methods of Identifying Pairs of Perturbed Genes and Adapting Genes and Methods of Increasing Gene Expression

Aspects of the present disclosure provide pairs of genes in which RNA decay of the first gene in the pair induces upregulation of expression of a second gene. The first gene may be referred to as the perturbed gene. The second gene may be referred to as the adapting gene. In some embodiments, a perturbed gene and a corresponding adapting gene is selected from a perturbed gene and adapting gene pair set forth in Table 7. In some embodiments, the perturbed gene is ACTG1 and the adapting is one or more genes set forth in Table 8. In some embodiments, a perturbed gene comprises one or more of the following motifs: CATCCCT (SEQ 89);

(SEQ 90)
ATCCCTG;
(SEQ 91)
CCCATCC;
(SEQ 92)
CACTTCC;
(SEQ 93)
TCCCTTC;
(SEQ 94)
TCCCATC;
(SEQ 95)
TCCCCTC;
(SEQ 96)
CCCTTCT;
(SEQ 97)
CCCTCTT;
and/or
(SEQ 98)
CCTACCC.

Further aspects of the present disclosure provide methods of identifying corresponding adapting genes for one or more perturbed genes. In some embodiments, a method of identifying corresponding adapting genes for a perturbed genes comprises introduction of one or more frameshifts into a perturbed gene in a cell and identifying corresponding adapting genes as genes whose expression is increased relative to 1) cells in which the perturbed gene is unaltered and 2) cells in which the one or more perturbed genes is knocked down.

The present disclosure encompasses use of any of the proteins and/or nucleic acids disclosed herein to increase expression of one or more genes. Any of the proteins and/or nucleic acids disclosed herein may be administered to a cell, tissue, organ and/or subject to increase expression of a gene. As a non-limiting example, a trigger nucleic acid can be used to increase expression of one or more adapting genes. In some embodiments, a trigger nucleic acid that comprises a portion of an mRNA transcript that encodes a perturbed gene disclosed herein is administered to a cell, tissue, organ, and/or subject to increase expression of a corresponding adapting gene disclosed herein. In some embodiments, a trigger nucleic acid that is complementary to one or more regions of an antisense transcript of a perturbed gene disclosed herein is administered to a cell, tissue, organ, and/or subject to increase expression of a corresponding adapting gene disclosed herein. In some embodiments, a perturbed gene and a corresponding adapting gene is selected from a perturbed gene and adapting gene pair set forth in Table 7. In some embodiments, the perturbed gene is ACTG1 and the adapting is one or more genes set forth in Table 8.

In some embodiments, a method of increasing expression of an adapting gene comprises inducing RNA decay of the mRNA of a corresponding perturbed gene. In some embodiments, a method of increasing expression of an adapting gene comprises introducing frameshift mutations into a corresponding perturbed gene. As a non-limiting example, a nuclease-active CRISPR/Cas9 system with two guides could be used as described in Example 4. In some embodiments, a perturbed gene and a corresponding adapting gene is selected from a perturbed gene and adapting gene pair set forth in Table 7. In some embodiments, the perturbed gene is ACTG1 and the adapting is one or more genes set forth in Table 8.

### Methods of Treatment and Uses

The present disclosure encompasses use of any of the proteins and/or nucleic acids disclosed herein to treat a disease. Any of the proteins and/or nucleic acids disclosed herein may be administered to a cell, tissue, organ and/or subject to treat a disease in which an increase in expression of a gene of interest would be beneficial. In some embodiments, the disease is not characterized by aberrant expression of a gene of interest. Any of the proteins and/or nucleic acids disclosed herein may be administered to a cell, tissue, organ and/or subject to treat a disease characterized by a decrease in expression of a gene of interest. In some embodiments, a ribonucleoprotein complex (e.g., a ribonucleoprotein complex comprising a Cas protein, a ILF3 sequence, and/or a fusion protein and a ribonucleic acid) is administered. In some embodiments, the engineered nucleic acid is a guide RNA or a trigger nucleic acid. In some embodiments, an engineered nucleic acid encoding a Cas protein, an ILF3 sequence and/or fusion protein sequence is administered. In some embodiments, an expression vector encoding a trigger nucleic acid is administered. In some embodiments, a virus disclosed herein is administered. In some embodiments, a lipid nanoparticle disclosed herein is administered.

Aspects of the present disclosure provide methods of treating a disease characterized by a decrease in expression of a gene of interest by deactivating one or more antisense transcripts of the gene of interest to increase expression of the gene of interest. An antisense transcript may be deactivated by disrupting or preventing the interaction between the antisense transcript and the mRNA or promoting degradation of the antisense transcript.

Diseases characterized by a decrease in expression of a gene of interest include diseases in which one or more functional alleles of a gene of interest are lacking. In some embodiments, a disease characterized by a decrease in expression of a gene of interest is monogenic disease in which the lack of one or more functional alleles of a gene causes the disease. In some embodiments, a protein and/or nucleic acid disclosed herein may be used to upregulate the wild-type allele in case of diseases caused by heterozygous mutations. In some embodiments, a protein and/or nucleic acid disclosed herein may be used to upregulate a paralog of a gene of interest for diseases caused by homozygous mutations. In some embodiments, a protein and/or nucleic acid disclosed herein may be used to upregulate a mutant allele that encodes a gene product that retains at least some functional activity of a normal counterpart.

In some embodiments, a disease characterized by a decrease in expression of a gene of interest is a haploinsufficiency disorder. Non-limiting examples of haploinsufficiency disorders include familial hypercholesteremia, autosomal dominant polycystic kidney disease (APKD), neurofibromatosis, and hypertrophic cardiomyopathy.

In some embodiments, a disease characterized by a decrease in expression of a gene of interest or aberrantly low activity of a protein of interest is an autosomal recessive disorder, in which two mutated alleles of a gene are required to produce a phenotype. In some embodiments, an autosomal recessive disorder is caused by a mutation in a gene that has a paralog, including but not limited to Duchenne muscular dystrophy (DMD), sickle cell anemia, hemochromatosis, alpha-1 antitrypsin deficiency, and beta thalassemia intermedia. For example, DMD is often caused by mutations in the dystrophin gene. Utrophin is a paralog of DMD, which can partially rescue the DMD phenotype in animal models. See, e.g., Tinsley et al., Nat. Med. 1998; 4:1441-1444. It has also been observed that expression of the fetal gene paralog γ-globin may be used to ameliorate sickle cell anemia or β-globin disease, sickle cell disease and β-thalassemia. Hemochromatosis is commonly caused by missense mutations in HFE, which has a paralog (HFE2). Alpha-1 Antitrypsin Deficiency is often caused by a missense mutation in the SERPINA1 gene, which has several paralogs including SERPINA4.

In some embodiments, a disease characterized by a decrease in expression of a gene of interest is a cancer. In some embodiments, a protein and/or nucleic acid disclosed herein may be used to upregulate a tumor suppressor gene.

### EXAMPLES

In order that the present disclosure may be more fully understood, the following examples are set forth. The synthetic and biological examples described in this application are offered to illustrate the compounds, pharmaceutical compositions, and methods provided herein and are not to be construed in any way as limiting in their scope.

### Example 1: Identification of ILF3 Domains Involved in Upregulation of Gene Expression

To determine the contribution of various ILF3 domains on upregulation of gene expression, ACTG1 KO;ILF3 KO knockout cells were used. qPCR analysis was conducted to evaluate the ability of two different ILF3 isoforms (NF90 and NF110, Table 1) and ILF3 with various domains knocked out to rescue ACTG2 upregulation in ACTG1 KO;ILF3 KO cells.

A schematic of the two different ILF3 isoforms (NF90 and NF110) with the domains is shown in FIG. 1A. The data show that NF110 rescued better than NF90 (FIG. 1B). Therefore, NF110 was used for the dcas13-NF110 fusions (Table 2). FIG. 1C shows qPCR analysis results similar to FIG. 1B but rescuing with NF110 where certain domains were deleted. In DZF-deleted cells, expression of dCas13-NF110deltaDZF rescued the ILF3 knockout. In FIG. 1C, the same methods were used except NF110 with certain domains deletions were used. The DZF deletion could still rescue very well, which suggests that having NF110deltaDZF could be used to increase gene expression (e.g., alone or in a fusion protein with a Cas protein that does not comprise nuclease activity toward target RNA), Additionally, Without wishing to be bound by any particular theory, the smaller size may be advantageous for packing inside a viral vector. qPCR results of the impact of expressing NF110 without a NLS domain in ILF3 knockout cells are shown in FIG. 1D.

### Example 2: Use of Trigger Screens to Identify and Design Engineered Nucleic Acids to Upregulate Gene Expression

Screens (referred to in this Example as trigger screens) were developed to identify RNA sequences which can be used to increase expression of genes of interest or paralogs thereof.

Fragments of ACTG1 was cloned into a non-sense mediated decay vector, and quantitative polymerase chain reaction (qPCR) analysis was performed to assess changes in transcriptional levels of ACTG2. The NMD transgene system was utilized as shown FIG. 2A. qPCR analysis of mRNA of ACTG2 and endogenous ACTG1 (detected by a primer binding to the 5′UTR which is missing in the NMD transgene) levels were measured upon inducing the ACTG1 NMD transgene cells with doxycycline relative to control GFP-2A-RFP NMD vector. The data show that the transgene upregulated ACTG1 and ACTG2 in an ILF3 dependent manner (FIG. 2B). qPCR analysis of endogenous SOX9 (FIG. 2C) or BDNF (FIG. 2D) expression upon inducing the SOX9 (FIG. 2C) or BDNF (FIG. 2D) NMD transgene MEFs (FIG. 2C) or HEK293 Ts (FIG. 2D) with doxycycline was also evaluated. The data show that NMD transgenes induced a transcriptional activation (TA) effect.

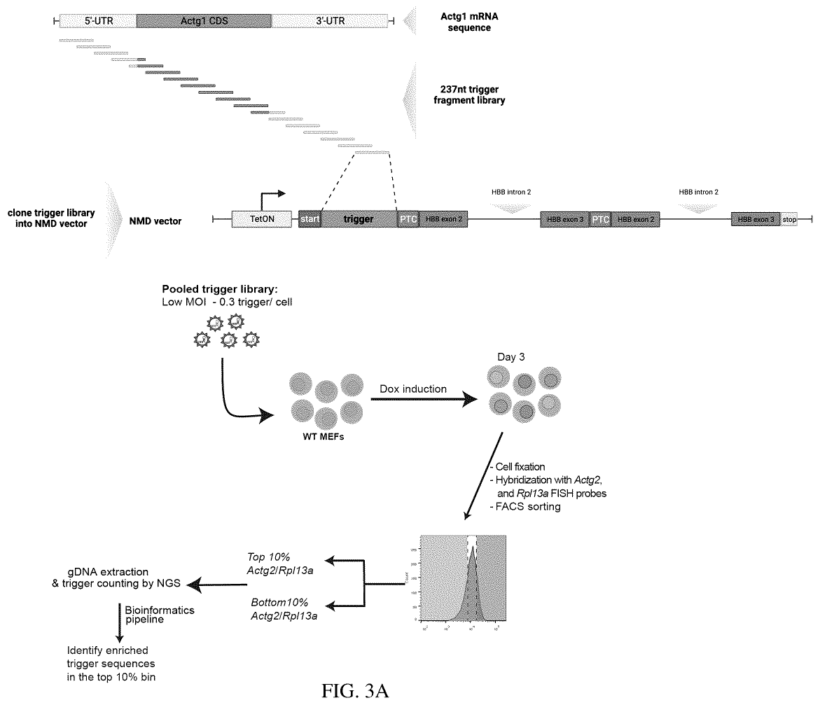

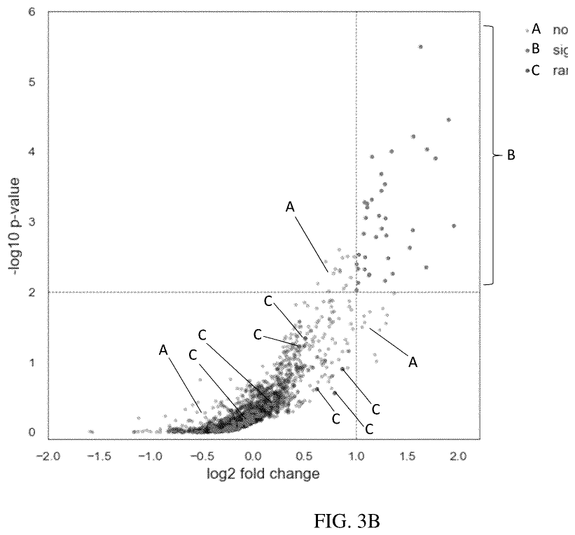

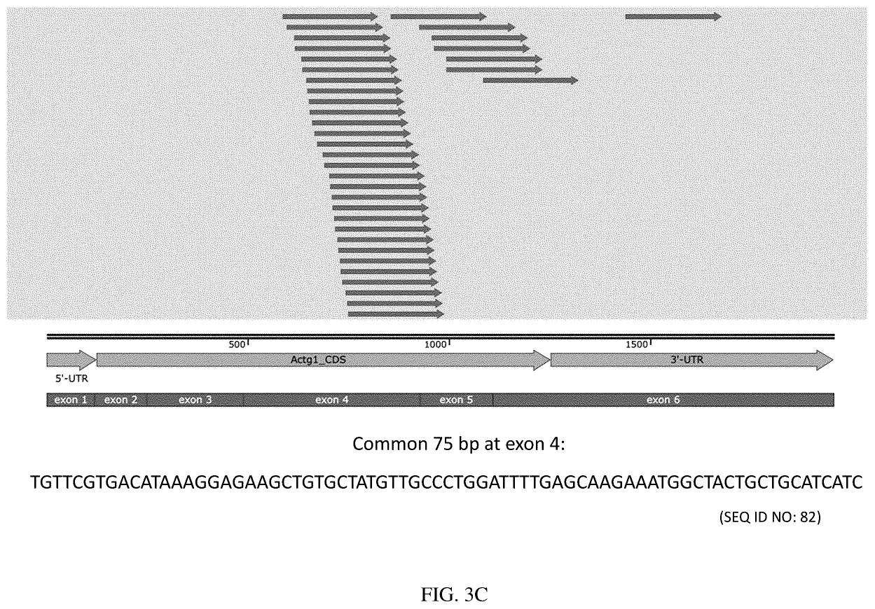

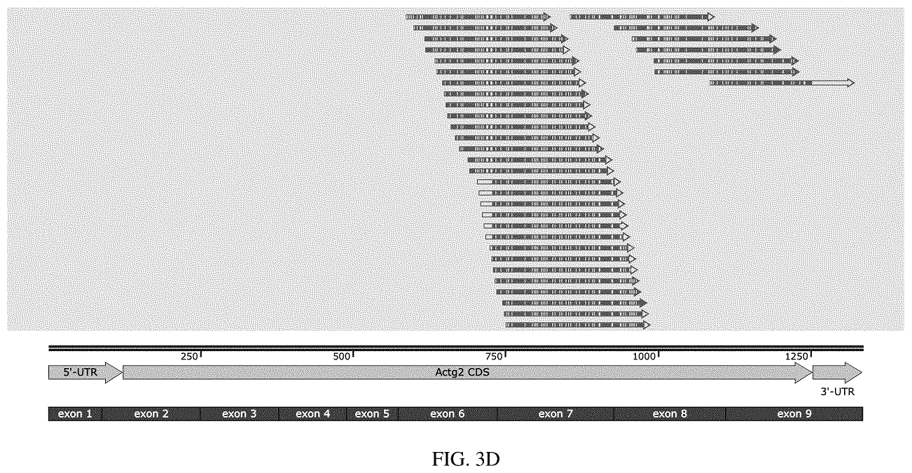

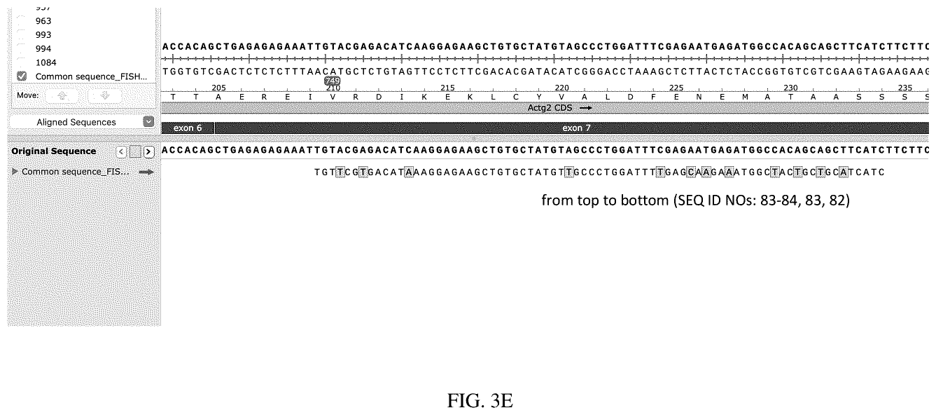

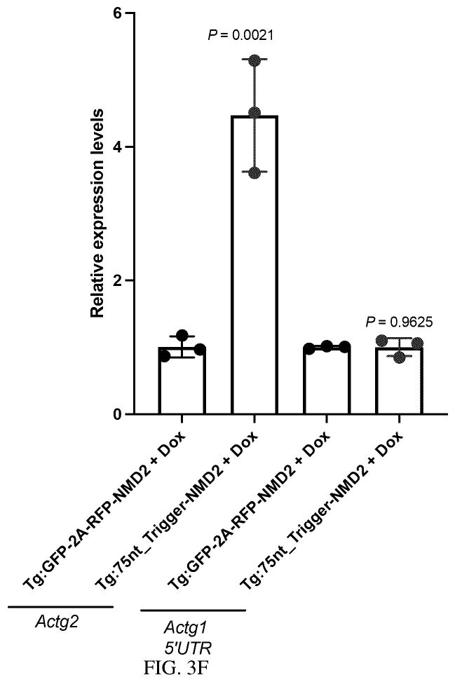

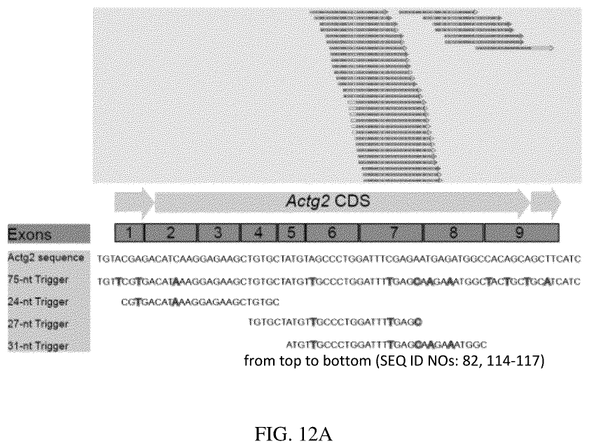

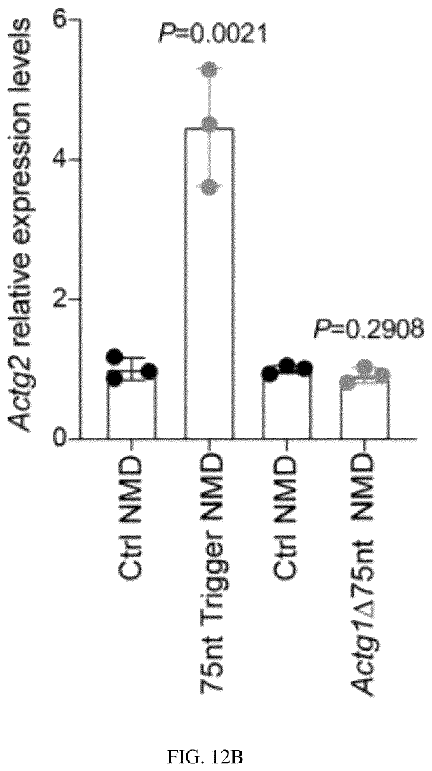

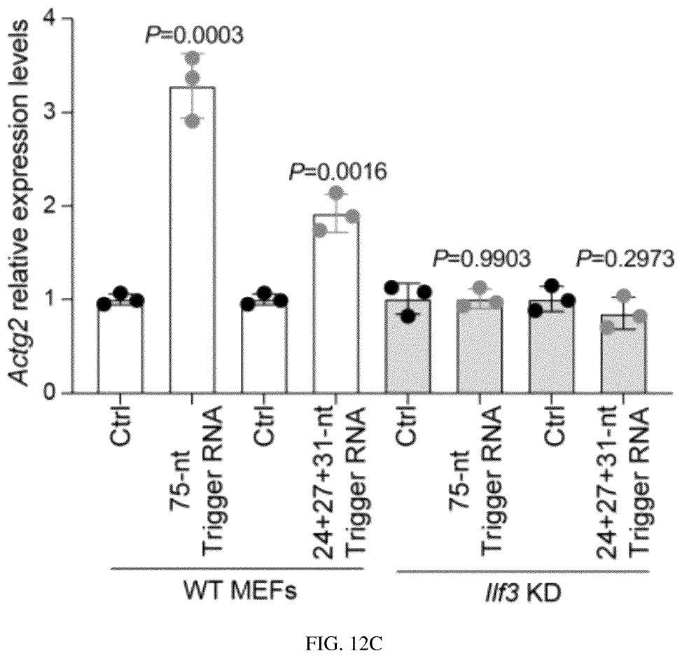

To identify fragments of ACTG1 that induce ACTG2 expression, FLOW-FISH analysis was performed according to the method shown in FIG. 3A. A volcano plot of log 2 fold change of enrichment of different triggers in the top 10% (ACTG2: Rpl13a expressing cells) over the bottom 10% and P-values from the trigger screen, with each point representing one trigger is shown in FIG. 3B. A diagram showing the map of the significant triggers to ACTG1 mRNA sequence is shown in FIG. 3C. The diagram shows that most significant triggers share a 75-nucleotide (nt) region. A diagram showing the map of the significant triggers to ACTG2 mRNA sequence is shown in FIG. 3D. The significant triggers shared extensive homology with ACTG2. Alignment of the identified 75-nucleotide region from the trigger screen to ACTG2 is shown in FIG. 3E and FIG. 12A. To validate the trigger screen results, the 75-nucleotide region was cloned into the NMD2 ptrex vector. qPCR analysis of endogenous ACTG2, ACTG1, and SOX9 expression upon inducing the 75-nt trigger NMD transgene with doxycycline was conducted. The results show that the 75-nt trigger caused a stronger upregulation of ACTG2 with no upregulation of ACTG1 or SOX9, suggesting its higher specificity (FIG. 3E). FIG. 3F shows qPCR analysis of endogenous ACTG1 and ACTG2 expression upon inducing the 75-nt trigger NMD transgene with doxycycline relative to the control GFP-21-RFP NMD vector. In summary, the trigger screens identified a 75-nt region in ACTG1 that was sufficient to promote ACTG2 expression.

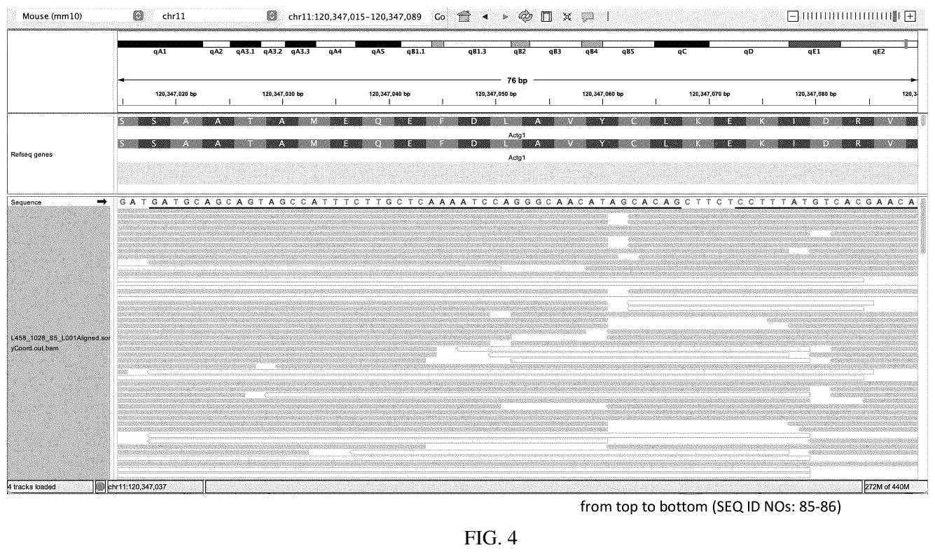

Without wishing to be bound by any particular theory, mRNA decay intermediates of one gene may guide ILF3 to genes exhibiting sequence similarity by hybridizing to antisense RNAs of a paralog gene and/or by hybridizing to antisense RNAs of the gene. To identify potential mRNA decay intermediates bound to ILF3, native RNA immunoprecipitation (RIP) of ILF3 from mouse embryonic fibroblasts was performed followed by RNA sequencing. A representative screenshot of reads obtained from the small RNA sequencing upon ILF3 native RIP and mapped to the 75-nucleotide region of ACTG1 is shown in FIG. 4. Each arrow is a sequenced RNA with grey arrows being unique matches and white sequences being multimappers. The term “multimappers” refers to there being multiple regions in the genome with the exact sequence of that RNA; for example, it could be ACTG1, ACTG2 or any other gene. RNAs for the RNA transfection assays were chosen from the RNAs appearing in the sequencing data, encompassing a range of 21 to 60 nucleotides.

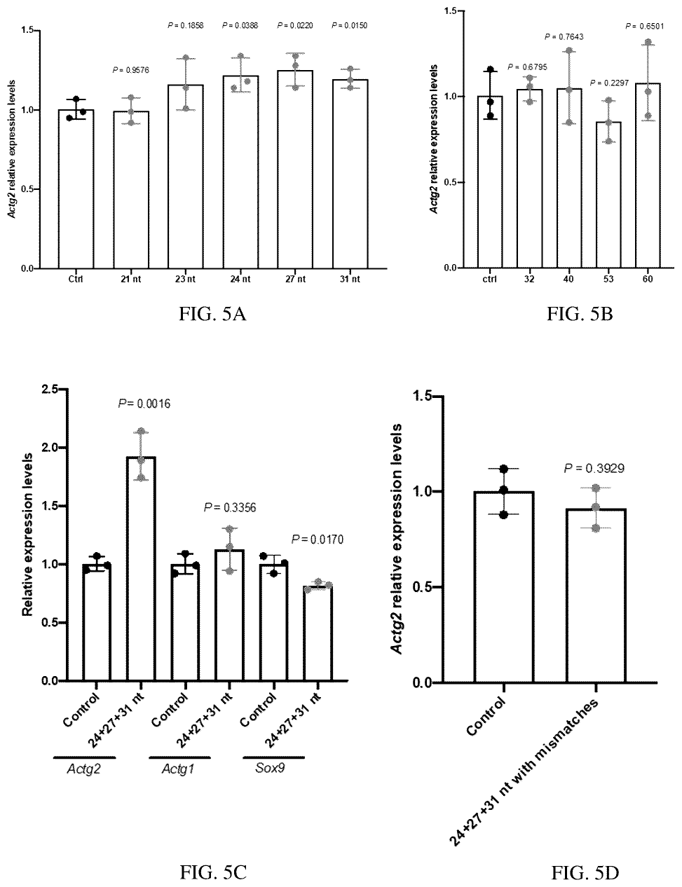

To assess TA-induced gene expression, the RNAs identified in the RIP assay and subsequent sequencing analysis described above were transfected into wild-type mouse embryonic fibroblasts (WT-MEFs). ACTG2 expression levels upon transfecting the indicated RNA relative to control were assessed by qPCR analysis. The data show that RNAs of 24, 27, and 31 nucleotides in length led to a significant, but mild upregulation of ACTG2 (FIGS. 5A-5B). qPCR analysis was conducted on the indicated genes (see FIG. 5C) upon transfecting a combination of the RNAs of 24, 27, and 31 nucleotides in length that showed mild upregulation when transfected alone. The data showed stronger upregulation of ACTG2 obtained with the combination of the three RNAs (FIG. 5C). The upregulation was specific, as both ACTG1 and SOX9 were not upregulated. The control used in FIG. 5C is the same as that of FIG. 5A. qPCR analysis of ACTG2 expression levels was conducted upon transfecting the combination of the three different RNAs but with mismatches. The mismatches appear to have led to prevention of upregulation of ACTG2 (FIG. 5D).

The 75-nucleotide region identified by the screen is sufficient to induce upregulation of ACTG2 (FIG. 12B). For FIG. 12B, the 75 nucleotide region was cloned as DNA into a plasmid vector and then transduced to cells. Transfection of cells with an RNA corresponding to the 75-nt trigger sequence (hereafter referred to as trigger RNA) led to a strong upregulation of ACTG2 in a fully ILF3-dependent manner (FIG. 12C).

Without wishing to be bound by any particular theory, the data suggests that ACTG2 expression could be induced by repressing antisense RNAs in the ACTG2 region corresponding to the 75-nucleotide region identified from the trigger screen. Without wishing to be bound by any particular theory, in some embodiments, naked RNAs could be degraded by cells, while antisense oligonucleotides (ASOs) may be more stable for in vivo approaches. It was next determined whether ASOs designed to target antisense RNAs in the 75-nucleotide region identified from the trigger screen could be used to increase ACTG2 expression.

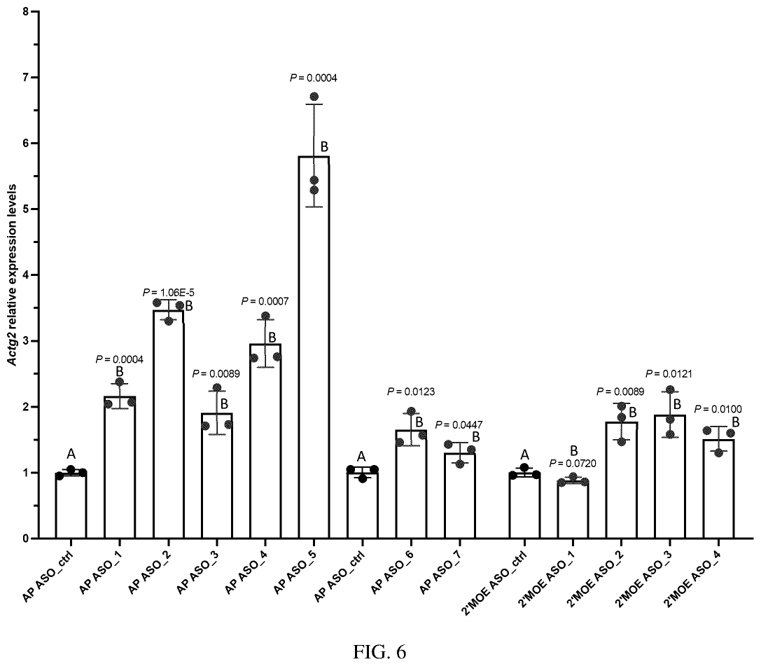

ACTG2 expression levels upon transfecting the indicated ASOs relative to control were assessed by qPCR analysis. ASOs were targeting the trigger screen identified region. The data show that most ASOs led to upregulation of ACTG2 with varying efficiencies (FIG. 6). AP (affinity plus) ASOs led to stronger upregulation effects on ACTG2 than MOE (2′-O-methoxyethyl base) ASOs. This was consistent with knowledge that AP ASOs cause a stronger knockdown effect on their target (in that case the antisense RNA). AP ASO_5 gave the strongest response and it is the only AP ASO that targets a 20-basepair perfect homology between ACTG1 (which the oligos used in the trigger screen were derived from), and ACTG2.

### Materials and Methods

NMD transgenes. A lentiviral vector (Poling et al., RNA Biology, 2017) allowing for the packaging and expression of transgenes containing introns was modified to include the HBB NMD exons and introns design used in the NMD2 vector from (Inglis et al., J Cell Sci, 2023) downstream of the GFP sequence (hereafter referred to as NMD2 ptrex plasmid). Cloning of full-length mouse ACTG1, mouse SOX9, or human BDNF was done from mouse or human cDNA and cloned between AgeI and NotI sites under a tetON promoter in the NMD2 ptrex plasmid. All ORF were cloned to have a premature stop codon truncating the ORF to avoid overexpressing the protein as NMD is often not 100% efficient. The resulting plasmid had puromycin as a marker and was used to produce lentiviruses in HEK cells. The original GFP NMD2 ptrex plasmid was used as a control. Plasmid sequences were verified by Sanger sequencing.

Lentivirus generation. Lentivirus was generated by transfecting HEK239T cells with the transfer plasmid and four packaging plasmids (for expression of VSV-G, Gag/Pol, Rev, and Tat) using TransIT-LT1 Transfection Reagent (Mirus Bio). Viral supernatant was harvested 2 days after transfection and filtered through 0.44 μm PES filters and/or frozen at −80° C. prior to transduction.

NMD transgene-stably expressing cells. The obtained lentiviruses were used to infect wild-type mouse embryonic fibroblasts (MEFs) for ACTG1 or SOX9 and HEK cells for BDNF. Seventy-two hours post infection, cells were treated with 5 ug/ml puromycin. Thereby, stable cells expressing the respective NMD transgene were obtained. At the end of the puromycin selection, cells were seeded in equal numbers in 24 well plates with 3 replicates for each condition and treated with 2 ug/ml doxycycline to induce the expression of the NMD transgene for 48-72 hours (controls were not treated with doxycycline).

Following that, RNA was isolated using TRIzol and at least 500 ng RNA was used for reverse transcription using the Maxima First Strand cDNA synthesis kit (Thermo). All reactions were performed in at least technical duplicates and the results represent biological triplicates. qPCR was performed in a CFX Connect Real-Time System (Biorad). qPCR primers were designed using Primer-BLAST. Fold changes were calculated using the 2^{-ΔΔC}t method. Hprt was used as the house keeping gene for data normalization. To detect the expression of the endogenous gene, primers binding to the 5′UTR or 3′UTR of the relevant gene were used, as the NMD transgene was composed of only the coding sequence.

Trigger library design and cloning. The trigger oligo pool was designed by tiling the entire mouse ACTG1 mRNA sequence (useast.ensembl.org/index.html) including the 5′- and 3′-UTRs into 237 nt triggers in 1 nt increments for the ACTG1 trigger library and in 10 nt increments for the control trigger library consisting of a randomly generated (faculty.ucr.edu/-mmaduro/random.htm) and iteratively optimized sequence with minimal mapability to the mouse genome. The overall synthesized oligo pool (Twist Biosciences) consisting of 1716 unique ACTG1 triggers and 86 unique control triggers have the following structure: 5′-PCR adapter-AgeI motif (ACCGGT)-Kozak sequence (GCCACC)-start codon (ATG)-237 nt trigger-stop codon (TAA)-NotI motif (GCGGCCGC)-PCR adapter 3′ and were cloned into the NMD2-ptrex-rtta-puro lentiviral vector. Two different PCR adapters were used for the ACTG1 trigger library and the control trigger library, which allowed for exclusive amplification of one of the libraries from the same oligo pool. Library cloning was guided by the protocol for Cloning of Pooled sgRNAs into Lentiviral Vector from the Weissman lab (weissman.wi.mit.edu/resources/Pooled_CRISPR_Library_Cloning.pdf) with adaptations of restriction enzymes used for insert/vector digestion and an E-Gel EX 2% Agarose followed by column purification using GeneJET MicroKit (Thermo Scientific) instead of polyacrylamide gels for insert purification. For oligo pool and library amplification, NEB next ultra II Q5 master mix 2× (NEB) was used throughout library preparation using the recommended PCR conditions. Library ligation was performed using T4 DNA ligase (NEB) at 16° C. for 16 hours, followed by ethanol precipitation overnight at −20° C. A 2100 bioanalyzer (Agilent) and a Qubit 4 Fluorometer (Invitrogen) were used throughout the library preparation to prevent library over-amplification. Due to the tiled sequences, the library is prone to recombination during cloning. This led to a series of transformations in RecA negative NEB Stable Competent E. coli (NEB) and colony Sanger sequencing and a large-scale transformation using Endura DUOs Electrocompetent Cells (BIOSEARCH) to avoid excessive recombination events during cloning. Endura cells were electroporated with trigger libraries and incubated for 14 hours at 37° C. The resulting library was purified using Plasmid Plus Maxi Kit (QIAGEN®) and amplified using staggered P5/P7 indexed primers for paired-end sequencing on the Miseq (ILLUMINA®) to confirm its balance.

Trigger screen. The trigger screen was performed in MEFs in two replicates. Lentiviral production of the pooled trigger library was scaled up according to the number of cells needed for the screen, and the virus volume that yields 30% infected cells was titrated in a 6-well plate. Seventy-two hours post transduction, cells were selected with 5 g/mL Puromycin Dihydrochloride (Thermo Scientific), and the percentage was determined relative to the untreated control. Around 12.6×10⁶ cells were infected in suspension and distributed across 3×15 cm plates for each cell line using a final concentration of 8 g/mL polybrene transfection reagent (Merck). Media was exchanged 24 hours post-transduction and maintained until 72 hours. Cells were subjected to Puromycin Dihydrochloride (THERMO SCIENTIFIC®) selection for 48 hours and maintained for a few more days to expand the cells. In total, 27 million cells were induced with 2 g/mL Doxycycline Hyclate (SIGMA®) and expanded in 15 cm plates with daily media exchange and DOX addition until 96 hours.

Flow-FISH. To get expression levels for the paralog ACTG2 in the trigger screen, Flow-FISH analysis was performed from the same pool of DOX-induced at 96 hours post dox induction. For the staining, the PrimeFlow™ RNA Assay kit (Invitrogen) was used with probes targeting ACTG2 and Rpl13a for cell size normalization. ACTG2 was stained using Alexa Fluor™ 647 labeled probes and Rpl13a mRNA was stained with Alexa Fluor™ 750 labeled probes. Cells were sorted on their A657 to A750 ratio, and the top and bottom 10% were sorted, centrifuged at 800 g for 5 minutes, and frozen at −80° C. until gDNA was isolated.

gDNA extraction from the trigger screen and sequencing. Frozen cell pellets were thawed and gDNA was isolated using the NUCLEOSPIN© Blood L kit (MACHEREY-NAGEL). gDNA was eluted from the column in 200 μL elution buffer, and 4×50 μL gDNA elution per sample was used for PCR amplification using P5/P7 primers, with each sample having a unique P7 index. The P5/iP7 primers allow amplification of the trigger cassette from the gDNA and to identification of triggers enriched in the top 10% relative to the bottom 10% of each replicate for both ACTG1 and ACTG2 samples. Amplicons were SPRI selected using an SPRI bead ratio of 0.5× and 0.8× from the initial PCR volume of 100 μL. The SPRI-selected amplicons were quantified on the Qubit 4 Fluorometer (Invitrogen) using the Qubit dsDNA BR Assay Kit (Thermo Fisher). In addition, a qPCR was run for P5/P7 containing DNA using the KAPA library quantification kit (Roche) and amplified libraries were prediluted to 2 pM according to the Qubit results. The picomolar concentration of each sample was measured. Based on the quantifications, each library sample was mixed pooled in equimolar ratios and prepared for Miseq using custom primers and the MISEQ® Reagent Kit v3 (ILLUMINA®).

Data analysis of the trigger screen. To obtain significantly enriched triggers, the dataset was demultiplexed and the paired reads were trimmed until the AUG start and TAA stop codon of each using cutadapt (cutadapt.readthedocs.io/en/stable/). Each paired read was mapped to the trigger library using a Python script (github.com/josephreplogle/CRISPRi-dual-sgRNA-screens), and significant triggers with a log 10 p-value of >2 and a log 2 fold-change (LFC) >1.0 were extracted using Python to visualize the data.

Native RNA immunoprecipitation. Native RIP for ILF3 was performed using the Magna Nuclear RIP (Native) RNA-Binding Protein Immunoprecipitation Kit (EMD Millipore) on mouse embryonic fibroblasts as per the manufacturer's protocol and using two ILF3 antibodies (ab92355, Abcam and BDB612155, BD biosciences). The pulled down RNA was used to generate small RNA sequencing libraries using the SMARter smRNA-Seq Kit for Illumina (Takara) as per the manufacturer's protocol. The obtained libraries were then sequenced on a NovaSeq SP illumine machine. Obtained reads were then mapped to the mouse mm10 genome to identify RNAs pulled down that were mapping to the 75-nt region identified from the trigger screen.

Trigger RNA transfection assays. RNAs were ordered from IDT (Table 3) except for the 75-nt trigger. Of each RNA except for the 75 nt RNA, 200 pmol was transfected into wild-type MEFs using SG Cell Line 4D-Nucleofector™ X Kit S (32 RCT) (LONZA®) as per the manufacturer's protocol, except that each transfection was seeded into 6 different 96 wells post nucleofection. For the 75 nt RNAs, 140 pmol was used. Twenty-four hours post nucleofection, RNA was isolated using the TRIzol method referred to earlier. For the assays with mismatches, for the 24-nt RNA, all 7 As were converted to Cs, and for the 27 and 31 RNAs, all 10 or 8 Us, respectively, were converted to Cs, unless converting to a C corrected the already existing mismatch between the ACTG1 mRNA and ACTG2. In that case, they were converted to Gs.

The 75-nt trigger RNA was invitro transcribed from a dsDNA, made from annealed oligos, of the sequence GTGAATTGTAATACGACTCACTATAGGGATGTGTTCGTGACATAAAGGAGAAGCTG TGCTATGTTGCCCTGGATTTTGAGCAAGAAATGGCTACTGCTGCATCATCTAA (SEQ ID NO: 109) using the T7 mMESSAGE mMACHINE Transcription Kit (THERMOFISHER®). The control 75 nt RNA was transcribed from a dsDNA with the sequence GTGAATTGTAATACGACTCACTATAGGGATGCAATTTCAGCCCTCTTATCCTCGGCG TTGTGTGTCAAGTGACGTAGACCTAGATTGACTCTATGACGGTATCTGCTAA (SEQ ID NO: 110).

ASO transfection. ASOs were designed by an IDT paid tool, and then ordered through IDT (Table 4). Of each ASO, 200 pmol was transfected into wild-type MEFs using SG Cell Line 4D-Nucleofector™ X Kit S (32 RCT) (Lonza) as per the manufacturer's protocol, except that each transfection was seeded into 6 different 96 wells post nucleofection. Twenty-four hours post nucleofection, RNA was isolated using the TRIzol method referred to earlier.

### Example 3: Using ILF3-Fusion Proteins to Modulate Gene Expression

Without wishing to be bound by any particular theory, the 75 nt region of Actg1 identified from the trigger screen in Example 2 shared extensive sequence homology with Actg2 and it is possible that Actg1 mRNA decay intermediates from that region may act on antisense RNAs in the corresponding region in Actg2 to promote gene expression. To determine whether recruitment of ILF3 to a region of the ACTG2 antisense RNA corresponding to the 75-nucleotide region identified in the trigger screen could be used to increase ACTG2 expression, ILF3 was fused to a Cas13 protein to produce a dCas13-ILF3 fusion protein that does not comprise nuclease activity toward target RNA. dCas13 refers to a Cas13 that does not comprise nuclease activity toward target RNA. Guide RNAs (gRNAs) targeting the region of the ACTG2 antisense RNA corresponding to the 75-nucleotide region identified in the trigger screen were also used.

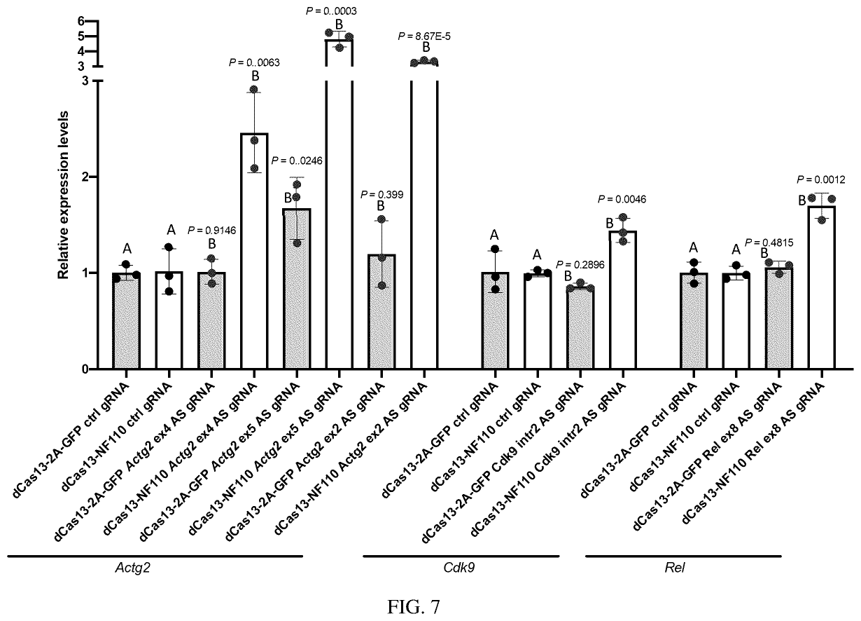

Transduction of wt MEFs (dCas13-2A-GFP control cell line and dCas-NF110 cell line) with the indicated gRNAs targeting ACTG2, Cdk9, and Rel was performed (see FIG. 7). Subsequently, quantitative polymerase chain reaction (qPCR) analysis of ACTG2, Cdk9, and Rel expression levels showed that targeting dCas13-NF110 to antisense RNAs promoted gene expression (FIG. 7).

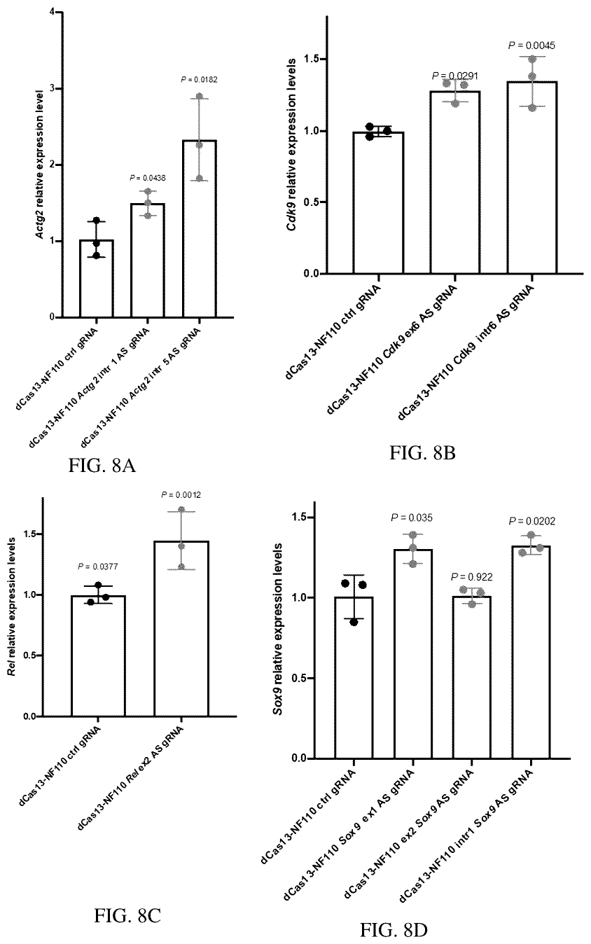

A variety of other gRNAs targeting antisense RNAs, ACTG2, Cdk9, Rel, and SOX9, were utilized with in cells expressing dCas13-NF110 as shown in FIGS. 8A-8D. Transduction of wt MEFs expressing dCas-NF110 cell line with the indicated gRNAs was performed. Subsequently, qPCR analysis of ACTG2, Cdk9, Rel, and SOX9 expression levels was conducted. The magnitude of upregulation varied depending on the gRNA. Without wishing to be bound by any particular theory, the magnitude of upregulation of gene expression may influenced by the region of antisense RNA targeted by a guide RNA and trigger screens may be useful in identifying the relevant regions to target.

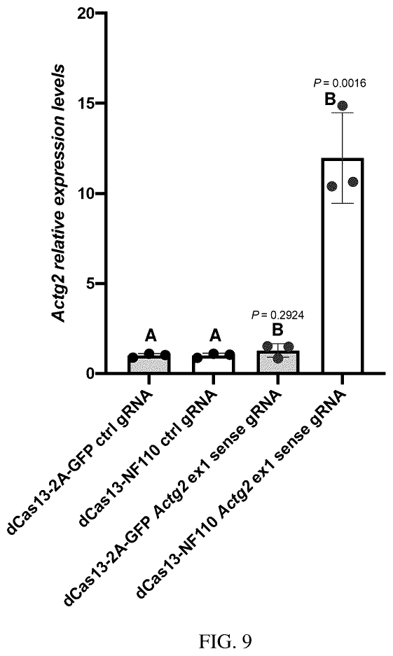

To determine whether sense RNA may be targeted to increase gene expression, cells were transduced with dCas13-NF110 with gRNAs targeting sense RNA. Quantitative polymerase chain reaction (qPCR) analysis was conducted to assess ACTG2 expression levels following transduction of wt MEFs expressing dCas13-2A-GFP control cell line and wt MEFs expressing dCas-NF110 cell line with the indicated gRNAs (see FIG. 9). The results showed that targeting dCas13-NF110 to ACTG2 sense RNA can also promote gene expression (FIG. 9). More examples of targeting dCas13-NF110 with a variety of gRNAs to ACTG2 sense RNA are shown in FIG. 10. Stronger magnitude of upregulation upon targeting sense RNAs compared to antisense was observed.

Without wishing to be bound by any particular theory, gRNAs targeting antisense RNAs designed based on the rank of an algorithm (cas13design.nygenome.org) sometimes led to strong upregulations in gene expression but sometimes not. As disclosed herein, a screening system was developed to identify RNA fragments that could be targeted to increase gene expression. In particular, in some embodiments, screens aimed at identifying, for a given gene, the shortest RNA sequences that can increase the expression levels of the adapting gene were developed. For example, for ACTG1, the screens were employed to investigate which parts of the ACTG1 mRNA can upregulate the adapting gene ACTG2 (the paralog).

The trigger screens allowed for identification of a 75-nucleotide region in ACTG1 mRNA that is sufficient to upregulate ACTG2. This 75-nucleotide region mapped to exon 7 of ACTG2. Then, dCas13 gRNAs were designed to target antisense RNAs in that region of exon 7 of ACTG2. Transducing such gRNAs to dCas13-NF110 expressing cells led to higher upregulation levels of ACTG2 than with any previously tested ACTG2 antisense gRNA.

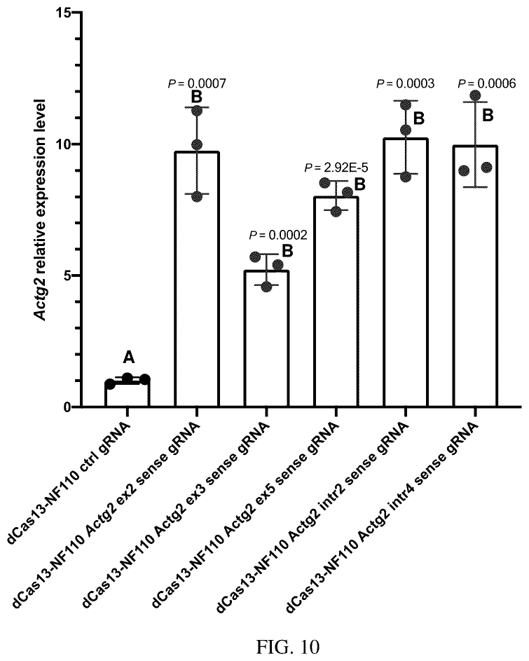

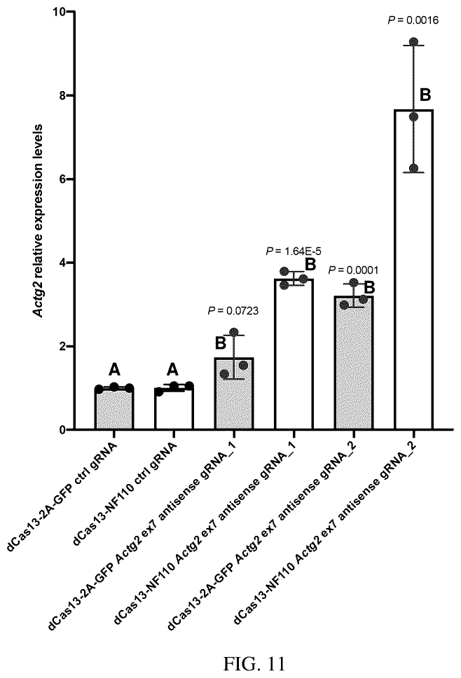

Using gRNAs identified from the above-described trigger screens, wt MEFs were transduced with dCas13-NF110 and dCas13-2A-GFP as a control. ACTG2 expression levels were measured by qPCR analysis following transduction. Targeting dCas13-NF110 to antisense RNAs in regions in Actg2 identified from a trigger screen led to stronger upregulations than those obtained by random designs (FIG. 11).

### Additional Materials and Methods

NF110 plasmid construction. Full length mouse NF110 was cloned from mouse cDNA and cloned with an XTEN80 linker at the C-terminus or N-terminus of dCasRx in the pXR002 lenti plasmid. The resulting plasmid had GFP as a marker and was used to produce lentiviruses in HEK cells. The original pXR002 plasmid (lacking the NF110 fusion) was used as a control. Plasmid sequences were verified by Sanger sequencing.

Lentivirus production. Lentivirus was generated by transfecting HEK239T cells with the transfer plasmid and four packaging plasmids (for expression of VSV-G, Gag/Pol, Rev, and Tat) using TransIT-LT1 Transfection Reagent (Mirus Bio). Viral supernatant was harvested 2 days after transfection and filtered through 0.44 μm PES filters and/or frozen at −80° C. prior to transduction.

Cell infection. The obtained lentiviruses were used to infect wild-type mouse embryonic fibroblasts (MEFs). Ninety-six hours post infection, cells expressing GFP were sorted, and thereby stable cells expressing dCas13-NF110-2A-GFP or the control dCas13-2A-GFP cells were obtained.

Guide RNAs and expression vector. CasRx guide RNAs were designed using the online tool: cas13design.nygenome.org/which uses an algorithm developed from Wessels et al., Nat Biotechnol, 2020. Guide RNAs targeting nascent or antisense RNAs of ACTG2, Cdk9, and Rel, or control gRNAs were ordered (Table 5). The gRNAs were cloned into the gRNA expression vector pLentiRNAGuide_001 between BsmBI sites. Those plasmids had puromycin as a selection marker. Plasmid sequences were verified by Sanger sequencing.

GFP fusions. The gRNA plasmids were then used to generate lentiviruses in HEK cells. The obtained lentiviruses were used to infect the wild-type MEFs expressing dCas13-NF110-2A-GFP or those expressing the control dCas13-2A-GFP cells. Seventy-two hours post infection, cells were treated with 5 ug/ml puromycin for 3 days to select for cells that express the gRNAs. At the end of the puromycin selection, cells were seeded in equal numbers in 24 well plates with 3 replicates for each condition.

Forty-eight hours post seeding (i.e., day 8 post infection with the gRNA plasmid), RNA was isolated using TRIzol and at least 500 ng RNA was used for reverse transcription using the Maxima First Strand cDNA synthesis kit (Thermo). All reactions were performed in at least technical duplicates and the results represent biological triplicates. qPCR was performed in a CFX Connect Real-Time System (Biorad). qPCR primers were designed using Primer-BLAST (Table 6). Fold changes were calculated using the 2^{-ΔΔC}t method. Hprt was used as the house keeping gene for data normalization.

### Example 4: Characterization of ILF3's Role in Upregulation of a Broad Range of Transcriptional Activation (TA) Targets

To determine whether ILF3 is required more generally for TA-mediated gene induction, and to systematically identify examples of TA, two complementary Perturb-seq strategies were used—CRISPR screens coupled to single-cell RNA-sequencing to analyze effects of gene perturbation (see, e.g., Dixit et al. Cell. 2016 Dec. 15; 167(7):1853-1866.e17 35-37; Adamson et al. Cell. 2016 Dec. 15; 167(7):1867-1882.e21; Datlinger et al. Nat Methods. 2017 March; 14(3):297-301)—in the human K562 cells. Perturbing genes using the nuclease-active CRISPR/Cas9 system (CRISPRn) and two closely spaced gRNAs per gene would introduce frameshift mutations resulting in PTCs that lead to NMD, which would trigger TA.

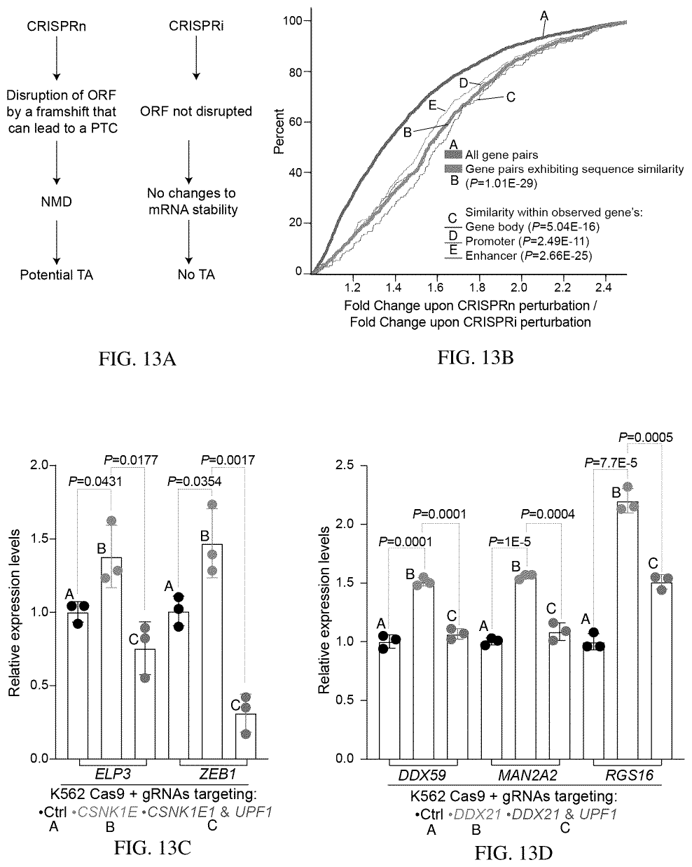

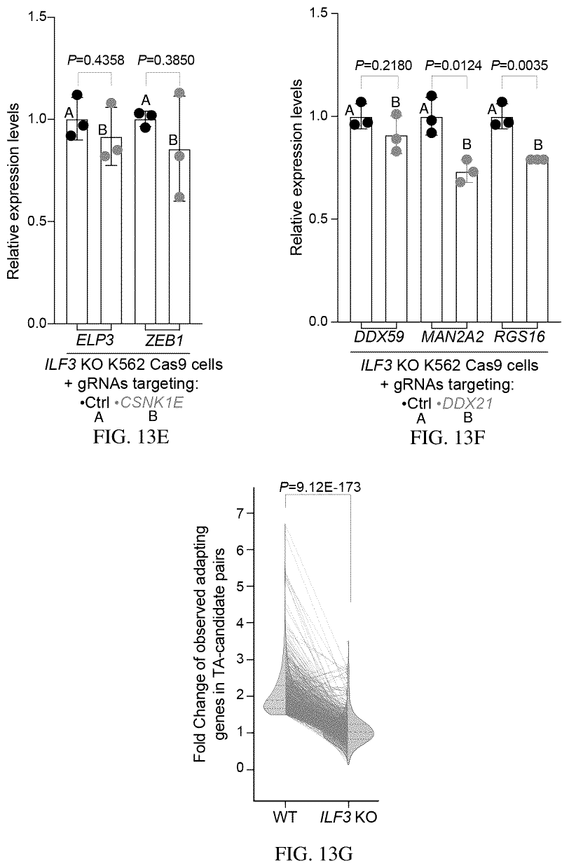

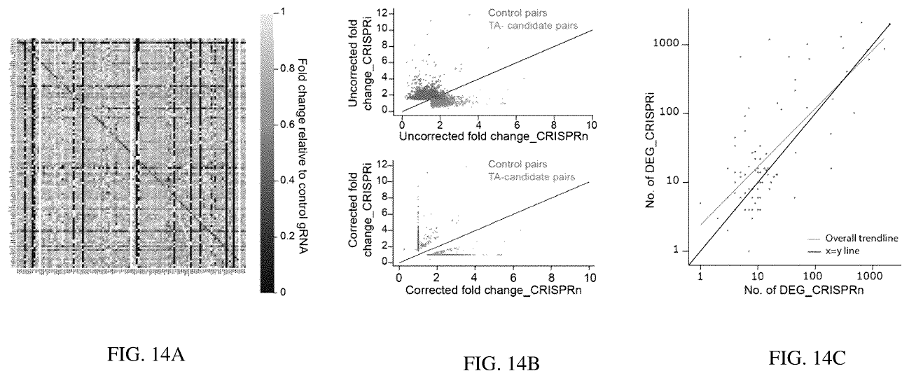

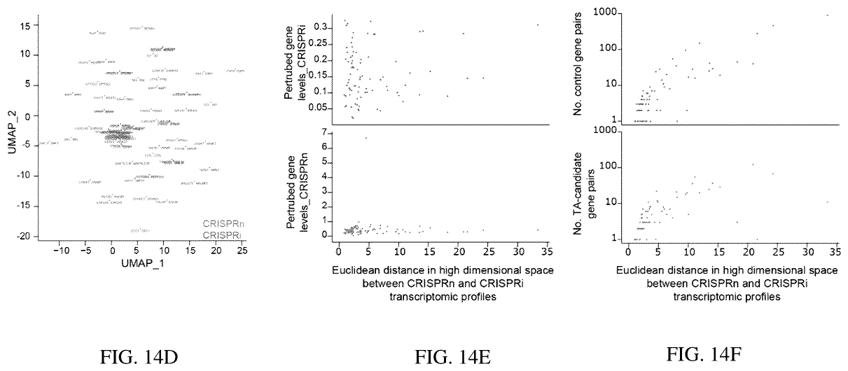

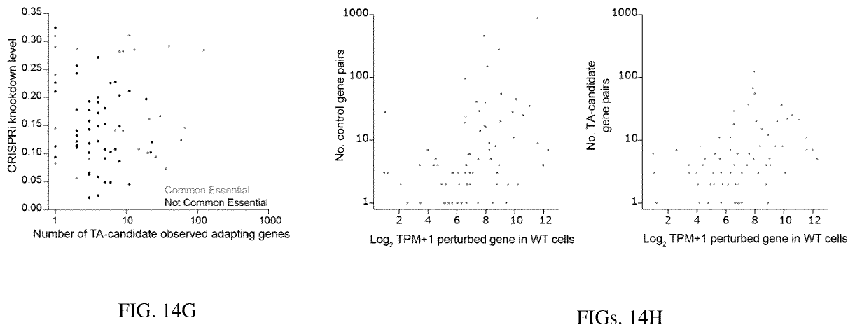

By contrast, perturbing genes using CRISPR-interference (CRISPRi) would repress transcription without inducing mRNA decay (see. e.g., Horlbeck et al. Elife. 2016 September 23:5:e19760), and thereby fail to induce TA but provide a control for transcriptional changes that are due to TA-independent loss of protein function effects. Comparing transcriptional responses between both methods of perturbation allow for the systemic interrogation of TA responses (FIG. 13A). A comprehensive dataset from a genome-wide CRISPRi Perturb-seq experiment in K562 was previously generated (see, e.g., Replogle et al. Cell. 2022 Jul. 7; 185(14):2559-2575.e28). Thus, complementary CRISPRn Perturb-seq experiment was performed, targeting 147 genes (selected to represent various gene categories, see methods)—for which 84 genes passed quality control for subsequent analysis. NMD for most of the targeted genes upon CRISPRn perturbation was observed to be efficient (FIG. 14A), confirming the efficacy of our CRISPRn libraries. Genes upregulated only upon CRISPRn perturbation of a given gene, but not with CRISPRi, represented potential adapting genes, and together with their perturbed gene were annotated as TA-candidate gene pairs. The pairs are shown in Table 7.

Pairs where the assessed gene was differentially expressed only upon CRISPRi-mediated perturbation were considered as a control group (hereafter referred to as control gene pairs, FIG. 14B, see methods). Typically, both CRISPRn and CRISPRi perturbations of a given gene similarly led to transcriptional responses that are a signature of successful perturbation of gene function. However, in addition to these global changes, transcriptional changes that were specific to CRISPRn, independent of several confounding factors were identified, and thus are candidate TA responses (FIG. 14C-14H).

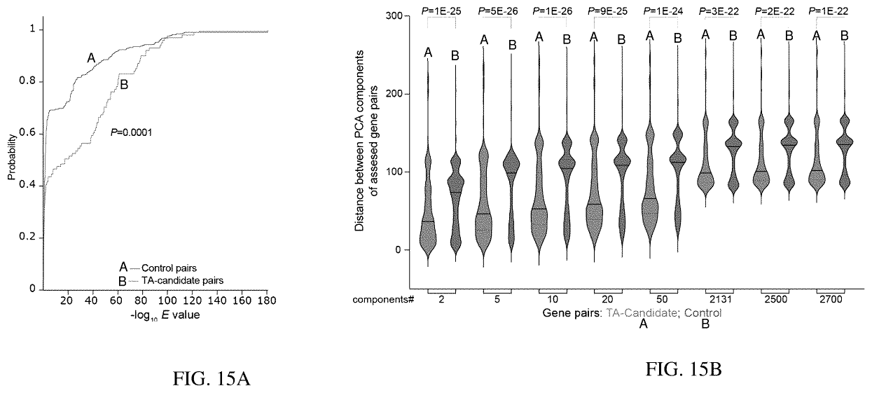

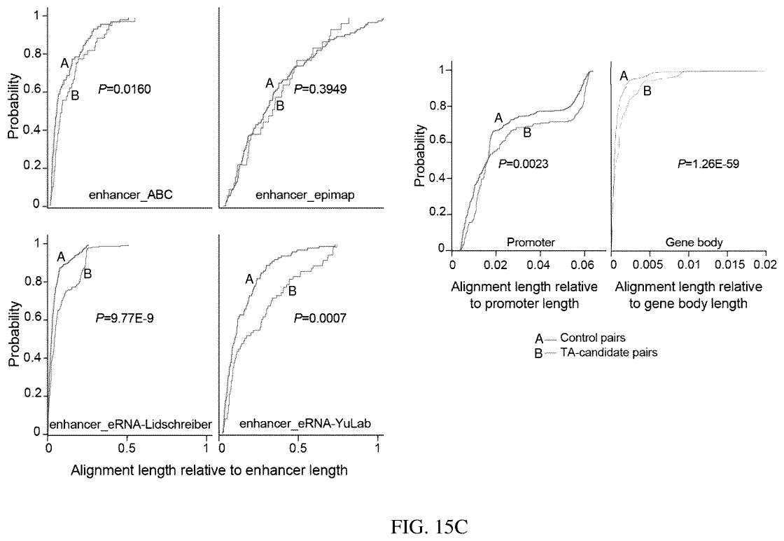

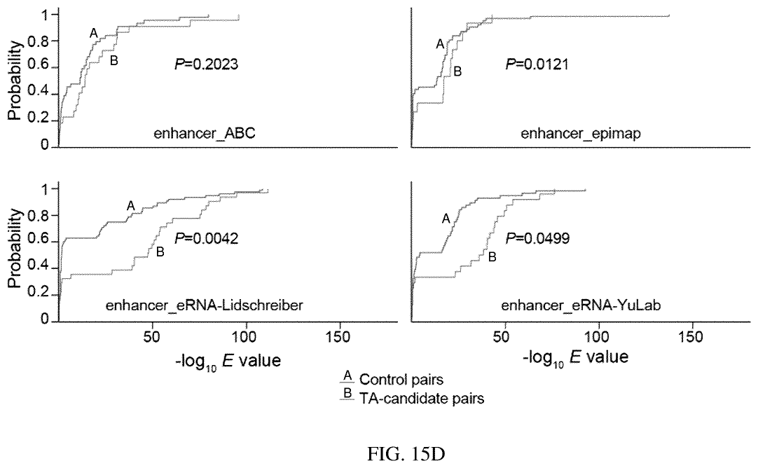

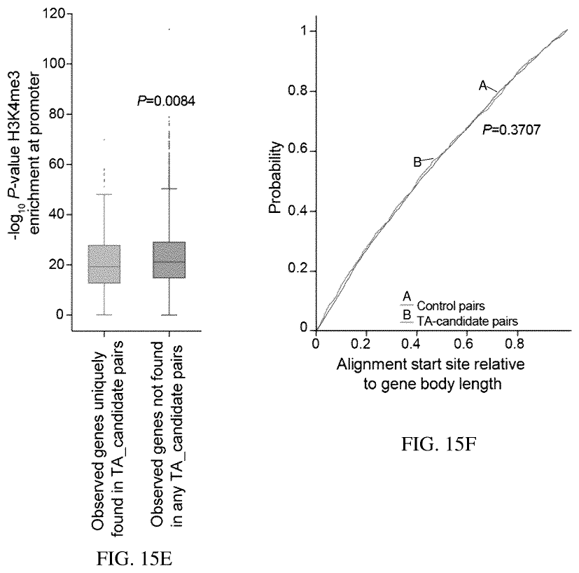

It was determined that genes exhibiting sequence similarity with the perturbed gene's mRNA were more likely to exhibit CRISPRn-specific upregulation (FIG. 13B), and TA-candidate gene pairs exhibited higher sequence similarity than control pairs (FIG. 15A). As another positive control, genetic inactivation of UPF1 in two randomly-selected perturbed genes, CSNK1E and DDX21, showed that NMD, a prerequisite for TA, is required for the upregulation of the respective adapting genes within a given TA-candidate pair (FIGS. 13C and 13D). Notably, from a UK biobank exome-wide association study, Backman et al. Nature. 2021 November; 599(7886):628-634, variants within a given adapting gene and its respective perturbed gene in TA-candidate pairs, displayed more correlated association patterns to health-related traits relative to control pairs (FIG. 15B). These data indicate that, within the context of TA, adapting genes are more likely to influence similar pathways or functions as the perturbed genes. This dataset also allowed the identification of other factors that influence TA including: 1) preferences for longer sequence similarity between the perturbed and the observed adapting genes (FIG. 15C), 2) similarities with enhancers may also drive TA, especially enhancers with evidence of enhancer RNA (eRNA) transcription (FIGS. 15A, 15C, and 15D), and 3) amenability for COMPASS-complex mediated H3K4me3 deposition at promoter regions of the adapting genes (FIG. 15E). In addition, no evidence for positional preference for the location of the alignment when similarity lies within the adapting gene body was observed (FIG. 15F).

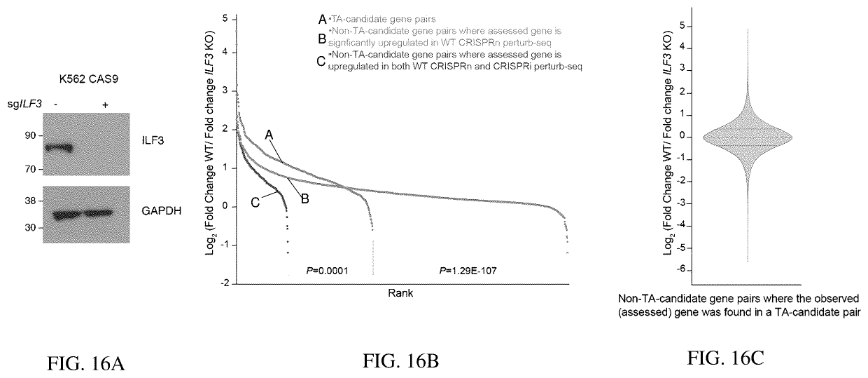

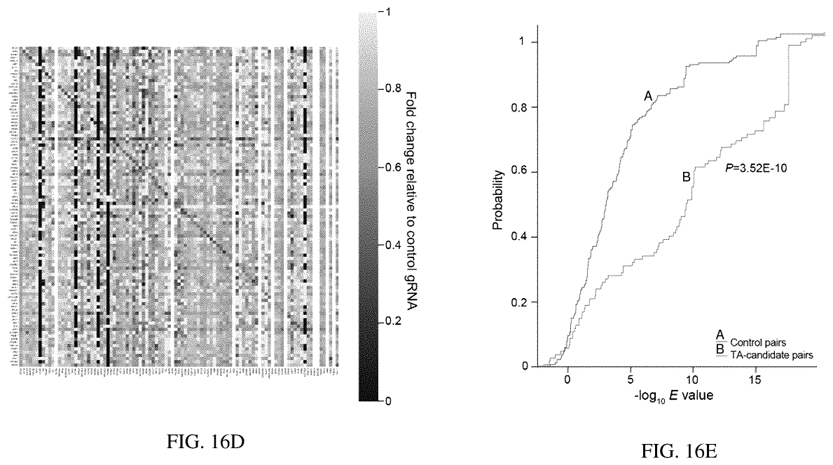

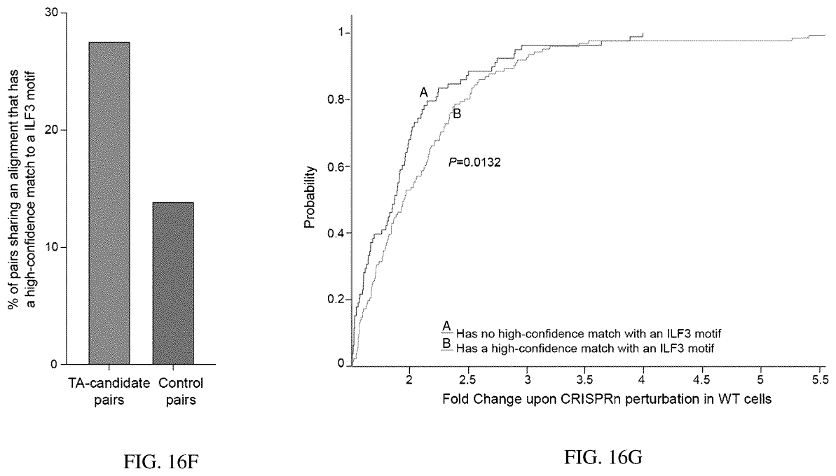

The set of TA-candidate pairs allowed the exploration of the requirement of ILF3 for TA in different models. Loss of ILF3 in the CSNK1E and DDX21 models abrogated the upregulation of the respective adapting genes (FIGS. 13E, 13F, and 16A). To investigate if ILF3 is required more generally for TA, the CRISPRn Perturb-seq experiment in ILF3 KO cells was repeated. Remarkably, adapting genes in 75% of the TA-candidate pairs were downregulated with loss of ILF3 (FIG. 13G and FIG. 16B), indicating that ILF3 is indeed a global regulator of TA. The decrease in expression levels of those adapting genes was specific to their respective perturbed gene within the TA-candidate pair, as their expression levels were largely unchanged with other perturbations (FIG. 16C). Similar to the Actg1 model, loss of ILF3 did not stabilize the mutant mRNAs of the perturbed genes (FIG. 16D), confirming that ILF3 acts downstream of mRNA decay in TA. For gene pairs that exhibit sequence similarity the alignment region from the perturbed gene (which represent potential TA-inducing mRNA decay intermediates) was compared with ILF3 motifs from eCLIP-seq data in WT K562s (see, e.g., Van Nostrand et al. Nature. 2020 July; 583(7818):711-71; and Feng et al. Mol Cell. 2019 Jun. 20; 74(6):1189-1204.e6 and SEQ ID NOs: 90-98). Alignment regions from TA-candidate pairs displayed higher sequence similarity to ILF3 binding motifs relative to control pairs, and a higher percentage of them had a high-confidence match to the motifs (FIGS. 16E and 16F). Of note, TA-candidate pairs where the alignment region had a high-confidence match to an ILF3 motif displayed higher upregulation levels of the respective adapting gene relative to those without (FIG. 16G). Taken together, the data demonstrate that ILF3 is a key regulator of TA. The data also characterized several ILF3 motifs in perturbed genes.

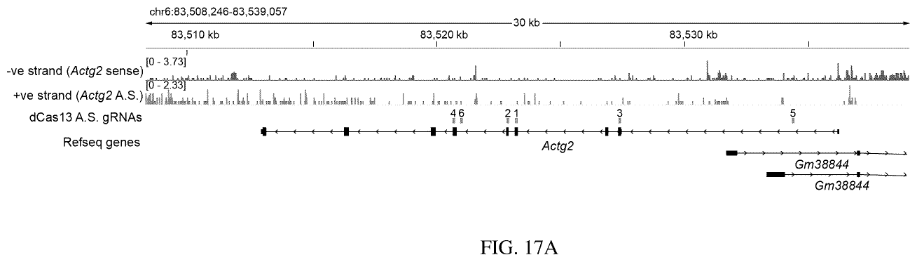

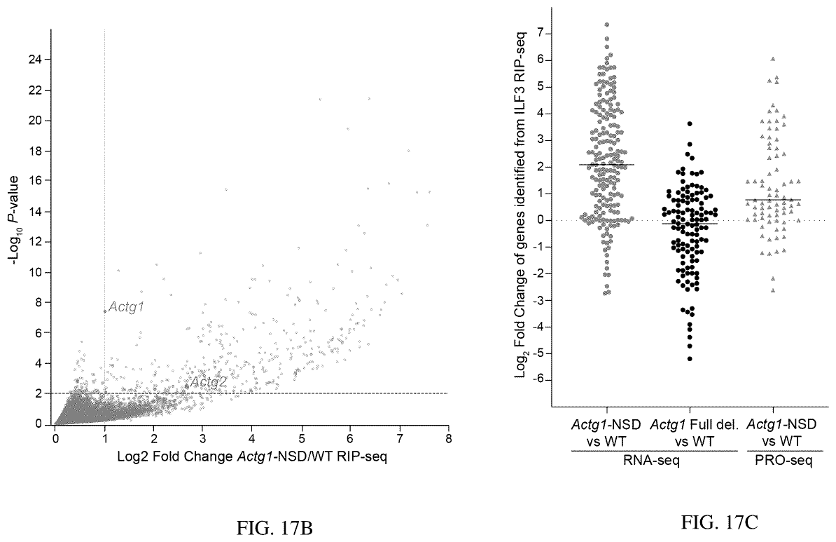

Precision nuclear run-on sequencing (PRO-seq) analysis revealed the presence of antisense transcription at the Actg2 locus (hereafter referred to as Actg2 antisense RNAs) (FIG. 17A). Thus, cross-linked ILF3 RNA immunoprecipitation followed by sequencing (RIP-seq) on nuclear fractions of WT and Actg1-NSD MEFs will allow the identification of genes to which ILF3 is preferentially recruited. ILF3 was found to be more recruited to Actg2 in the Actg1-NSD cells relative to WT (FIG. 17B and Table 8). Notably, ILF3 was more strongly recruited to other genes, including Actg1, in the Actg1-NSD cells. These genes showed a strong propensity to be upregulated in Actg1-NSD cells relative to WT, and not in Actg1 full locus deletion cells where TA does not occur (see, e.g., El-Brolosy et al. Nature. April; 568(7751):193-197) (FIG. 17C), indicating that such genes are TA targets.

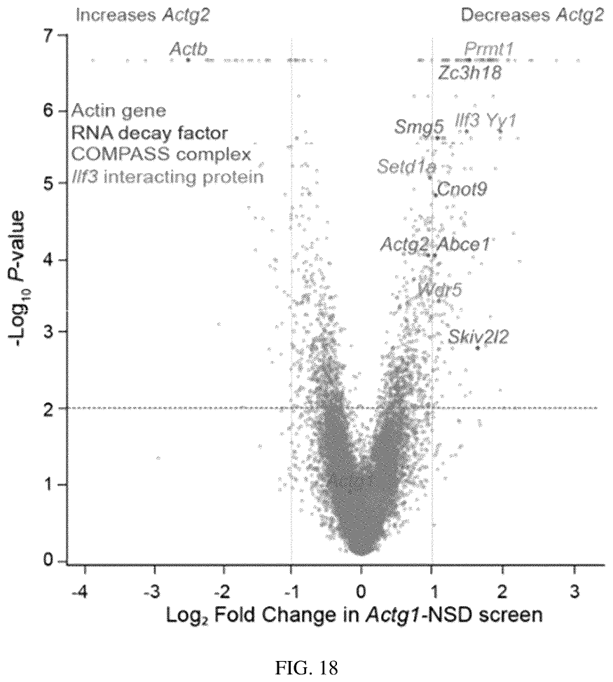

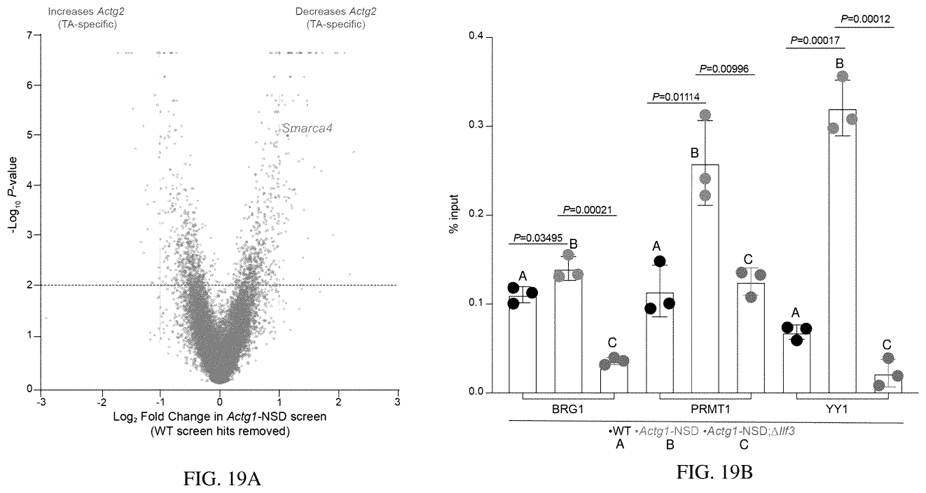

The CRISPR screen coupled to single-cell RNA-sequencing uncovered novel epigenetic modulators of TA. In addition to the previously-identified COMPASS complex (see, e.g., El-Brolosy et al. Nature. April; 568(7751):193-197 and Ma et al. Nature. April; 568(7751):259-263), the screen identified the ILF3 interactors and transcriptional activators PRMT1 and YY1 (see, e.g., Rezai-Zadeh et al. Genes Dev. 2003 Apr. 15; 17(8):1019-29; Chaumet et al. Biochimie. 2013 June; 95(6):1146-57; and Yao et al. Genome Med. 2021 Oct. 4; 13(1):154) (FIG. 18C), and BRG1 (encoded by Smarca4), the catalytic subunit of the SWI/SNF complex that increases chromatin accessibility (see, e.g., Centore et al.) (FIG. 19A). Of note, PRMT1 was reported to recruit BRG1 (Yao et al. Genome Med. 2021 Apr. 14; 13(1):58). ChIP experiments revealed ILF3-dependent enrichment of BRG1, PRMT1 and YY1 at Actg2 TSS in Actg1 NSD cells (FIG. 19B). The identification of the SWI/SNF complex explains the previously observed increased chromatin opening at adapting genes' loci (El-Brolosy et al. Nature. April; 568(7751):193-197 and Ma et al. Nature. April; 568(7751):259-263). Taken together, these data indicate that ILF3 is recruited to adapting genes' loci to increase gene expression through recruitment of epigenetic modifiers and enhancing transcription initiation and elongation.

### Materials and Methods

RIP-seq. Cross-linked RIP was performed using the Magna Nuclear RIP (Cross-Linked) Nuclear RNA-Binding Protein Immunoprecipitation Kit (Millipore Sigma) while native RIP was performed using the Magna Nuclear RIP (Native) Nuclear RNA-Binding Protein Immunoprecipitation Kit (Millipore Sigma) according to the manufacturer's protocol using at least 2×10⁷ WT or rescued Actg1-NSD MEFs per replicate. For the cross-linked RIP, fixed nuclei were subjected to sonication using Bioruptor (Diagenode) to generate fragments of 200-600 bp in size prior to IP. Enriched ‘input’ samples were generated by reserving 10% of the starting lysate; the remaining volume was subjected to IP using the with 10 μg Anti-ILF3 antibody (abcam; ab92355 and BD Biosciences; Clone 21/DRBP76) coated onto protein A/G magnetic beads as described in the Magna RIP technical manual. RNA purified from both IP and input samples was concentrated by ethanol precipitation and resuspended in equivalent volumes of RNase-free water to be used to generate RNA-seq libraries. For the cross-linked total RNA RIP-seq, sequencing libraries were generated using 10 ng of RNA from input and IP samples using the SMARTer® Stranded Total RNA-Seq Kit v2—Pico Input Mammalian (Clontech). RNA sequencing was performed on a NovaSeq S1 instrument (Illumina), resulting in an average of 43 million reads per library, with 50×50 bp paired-end setup. Reads were then trimmed followed by mapping to Ensembl mouse genome version mm10 (GRCm38) as described above. The number of reads aligning to genes were counted with featureCounts with the following parameters -B -C -s 0 -t exon, where only reads mapping at least partially inside exons were admitted, and these reads were aggregated per gene. Reads overlapping with multiple genes or aligning to multiple regions were excluded. Differentially expressed ILF3 binding in rescued Actg1-NSD MEFs vs WT was identified using DESeq2 v.1.14.158 as described in support.bioconductor.org/p/61509/. Genes with a baseMean >30, Log₂FoldChange >1 with P value <=0.01 (DeSeq) were classified as significantly differentially binding between Actg1-NSD and WT cells. Experiments were done using three biological replicates. For small RNA native RIP-seq, 10 ng of RNA was used to generate small RNA-seq libraries using the SMARTer smRNA-Seq Kit (Clonetech). RNA was treated with T4 Polynucleotide Kinase for 1 hr prior to library preparation to capture various potential mRNA decay intermediates that may not have a 5′P or 3′OH. RNA sequencing was performed on a NovaSeq S1 instrument (Illumina), with 150×150 bp paired-end setup (only Read 1, however, was used in downstream analysis). Cutadapt was then used to trim Read 1 using the following criteria m 15 -u 3 -a AAAAAAAAAAAAAAA as per the kit's manufacturer's recommendation followed by mapping to Ensembl mouse genome version mm10 (GRCm38) as described above. The generated BAM files were then used to identify RNAs associated with the 75-nucleotide trigger region.

CRISPRn perturb-seq library design and cloning. A dual gRNA CRISPRn library targeting 147 genes that included 10 negative-control non-expressed genes: MAGEA5, FOLH1B, TBC1D3B, SPATA31C2, ZNF806, and 5 olfactory receptors (OR4F29, OR1F1, OR2C1, OR3A1 and OR3A2), in addition to 5 pairs of non-targeting control sgRNAs was designed. The genes targeted spanned a wide range of gene ontology terms that included subsets of: (i) orthologs of genes targeted in previous genetic compensation studies (see, e.g., El-Brolosy et al. Nature. April; 568(7751):193-197 and Ma et al. Nature. April; 568(7751):259-263) (ii) genes identified to have stronger growth effects when targeted by CRISPRn versus CRISPRi and vice versa as identified in screen performed in a previous study, Hein et al. Nat Biotechnol. 2022 March; 40(3):391-401; (iii) Cancer Dependency Map common essential genes as defined in the year 2020, Quarter 1 (iv) non-essential genes (v) genes that are the control of bi-directional promoters (vi) 10 negative-control non-expressed genes as CRISPRn double-stranded breaks control (vii) non-targeting control sgRNAs accounting for 5% of the total library; the library was designed to include 9-10% control gRNAs (negative control and non-targeting gRNAs). To increase the potential of having an out-of-frame mutation that will elicit NMD, and to be on-par with the CRISPRi Perturb-seq dataset to which the data was going to be compared (see., e.g., Replogle et al. Cell. 2022 Jul. 7; 185(14):2559-2575.e28), a multiplexed CRISPRn library was constructed which targeted each gene with two unique sgRNAs expressed from tandem U6 expression cassettes in a single lentiviral vector (Replogle et al. Elife. 2022 December 28:11:e81856). The Human Improved Genome-wide Knockout CRISPR Library (Tzelepis et al. Cell Rep. 2016 Oct. 18; 17(4):1193-1205) and the Brunello library (Doench et al. Nat Biotechnol. 2016 February; 34(2):184-191) CRISPRn sgRNA library were used as a source of sgRNAs targeting each gene, with the optimal sgRNA pair targeting each gene selected to be the closest two sgRNAs to each other to avoid having large deletions that can influence TA responses. sgRNAs targeting within the first 150 nucleotides of an open reading frame was avoided as stop codons in these regions can escape nonsense-mediated decay (see, e.g., Lindeboom et al. Nat Genet. 2016 October; 48(10):1112-8). Cloning of the dual gRNA libraries with capture sequences for 3′ direct capture Perturb-seq into an sgRNA lentiviral expression vector (pJR101, Addgene #187241) was performed as described before see for e.g., Replogle et al. Cell. 2022 Jul. 7; 185(14):2559-2575.e28; Replogle et al. Elife. 2022 December 28:11:e81856; Replogle et al. Nat Biotechnol. 2020 August; 38(8):954-961; and weissman.wi.mit.edu/resources/2022_crispri_protocols/Protocol_1_dual_sgRNA_lib_cloning.pdf. Briefly, a two-step restriction enzyme digestion and ligation cloning of oligos into pJR101 was performed to maintain coupling of sgRNAs targeting the same gene. Oligos encoding the targeting regions of dual-sgRNA pairs were synthesized as an oligonucleotide pool (Twist Biosciences) with the structure: 5′-PCR adapter-CCACCTTGTTG (SEQ ID NO: 111)-targeting region A-gtttcagagcgagacgtgcctgcaggatacgtctcagaaacatg (SEQ ID NO: 112)-targeting region B-GTTTAAGAGCTAAGCTG (SEQ ID NO: 113)-PCR adapter-3′. Oligo pools were amplified, digested with BstXI/BlpI, and ligated into pJR101. To add an sgRNA constant region and U6 promoter to the vector, pJR89 (Addgene #140096) was BsmBI-digested and ligated into the intermediate library.

Perturb-seq. CRISPRn perturb-seq experiments were performed similar to the day 8 genome-wide CRISPRi perturb-seq (see., e.g., Replogle et al. Cell. 2022 Jul. 7; 185(14):2559-2575.e28) to allow for direct comparison of the two different datasets. The CRISPRn library was packaged into lentivirus in 293T/17 cells and K562 Cas9 cells were transduced via spinfection (1000 g) with polybrene (8 g/ml) with the target of obtaining an infection rate of ˜30%. Cells were maintained at a viability of >90%, a coverage of 1000 cells per library element, and a density of 250,000 to 1,000,000 cells/ml for the course of the experiment. Three days post transduction, cells were sorted to near purity by FACS (FACSAria2, BD Biosciences), using GFP as a marker for sgRNA vector transduction. Eight days post infection, the cells were measured to be 97% GFP+ (LSR2, BD Biosciences), >90% viable, and at a concentration of ˜800,000 cells/ml (Countess II, ThermoFisher). Cells were prepared for single-cell RNA-sequencing by resuspension in 1×PBS with 0.04% BSA as detailed in the 10× Genomics Single Cell Protocols Cell Preparation Guide (10× Genomics, CG00053 Rev C). Cells were then separated into droplet emulsions using the Chromium Controller (10× Genomics) with Chromium Single-Cell 3′ Gel Beads v3 (10× Genomics, PN-1000075) across 3 “lanes”/“GEM groups” following the 10× Genomics Chromium Single Cell 3′ Reagent Kits v3 User Guide with Feature Barcode technology for CRISPR Screening (CG000184 Rev C) with the goal of recovering ˜20,000 cells per GEM group before filtering. To perform the CRISPRn perturb-seq experiment in ILF3 knockout K562 cells, a dual sgRNA targeting ILF3 were cloned into an sgRNA lentiviral expression vector with mCherry as a selection marker and no for 3′ direct capture sequences. This lentivector was then packaged into lentivirus in 293T/17 cells and K562 Cas9 cells were transduced via spinfection as described above with the target of obtaining an infection rate of ˜30%. Three days post transduction, cells were sorted to near purity by FACS (FACSAria2, BD Biosciences), using mCherry as a marker for sgRNA vector transduction. The sorted cells were then directly transduced with the CRISPRn perturb-seq library using spinfection and handled as described above for the CRISPRn perturb-seq experiment done in WT K562 Cas9 cells. Eight days post infection with the CRISPRn perturb-seq library, the cells were measured to be 97% double positive for GFP and mCherry (LSR2, BD Biosciences). Cells were prepared for single-cell RNA-sequencing by resuspension in 1×PBS with 0.04% BSA as detailed in the 10× Genomics Single Cell Protocols Cell Preparation Guide (10× Genomics, CG00053 Rev C). Cells were then separated into droplet emulsions using the Chromium Controller (10× Genomics) with Chromium Single-Cell 3′ Gel Beads v3.1 (10× Genomics, PN-1000121) across 3 “lanes”/“GEM groups” following the 10× Genomics Chromium Single Cell 3′ Reagent Kits v3 User Guide with Feature Barcode technology for CRISPR Screening (CG000184 Rev C) with the goal of recovering ˜20,000 cells per GEM group before filtering. Loss of ILF3 did not seem to affect cell proliferation in MEFs, however it led to an observable cell proliferation phenotype in K562s which is consistent with its reported essentiality in K562s in the cancer dependency map porta (DepMap). For preparation of gene expression and sgRNA libraries, samples were processed according to 10× Genomics Chromium Single Cell 3′ Reagent Kits v3 or v3.1 User Guide with Feature Barcode technology for CRISPR Screening (CG000184 Rev C, CG000205). For sequencing, mRNA and sgRNA libraries were pooled to avoid index collisions at a 10:1 ratio. Libraries were sequenced on a NovaSeq (Illumina) according to the 10× Genomics User Guide. Following sequencing, reads were used as input to Cell Ranger for alignment. In total, 55846 cells were sequenced for the perturb-seq experiment in WT K562 Cas9 cells and 51371 for the ILF3 knockout cells.

Alignment, cell calling, and guide assignment. Cell Ranger 6.1.2 software (10× Genomics) was used for alignment of scRNA-seq reads to the transcriptome, alignment of sgRNA reads to the library, collapsing reads to UMI counts, and cell calling. The 10× Genomics GRCh38 version 2020-A genome build was used as a reference transcriptome. Reads from the sgRNA libraries were mapped with Cell Ranger. To account for differences in sequencing depths across GEM groups from the same experiment, reads were downsampled to produce a more even distribution of the number of reads per cell across gemgroups, with a threshold of 1000 reads per cell. Guide calling was performed with a Poisson-Gaussian mixture model as previously described. For each guide, the mixture model was fit 100 times, selecting the maximum likelihood model from among the fits. After guide calling, each cell was categorized according to its guide identities as representing a single genetic perturbation or a multiplet (which may arise from lentiviral recombination or multiple cell encapsulation during droplet generation). Only cells bearing two guides targeting the same gene were used for downstream analysis. Downstream analyses were performed in Python, using a combination of numpy, scipy, Pandas, scikit-learn, pomegranate, infercnvpy, pygenometracks, scanpy and seaborn libraries as described before see for e.g., Dixit et al. Cell. 2016 Dec. 15; 167(7):1853-1866.e17 and Replogle et al. Cell. 2022 Jul. 7; 185(14):2559-2575.e28.

Normalization of gene expression measurements and gene-level differential expression testing using the Mann-Whitney tests. The normalization processes used is similar to the one used for the CRISPRi genome-wide perturb-seq (see, e.g., Replogle et al. Cell. 2022 Jul. 7; 185(14):2559-2575.e28) and as described before (see, e.g., Dixit et al. Cell. 2016 Dec. 15; 167(7):1853-1866.e17) using control non-targeting sgRNAs. The normalized gene expression matrix for cells was then computed via UMI count normalization where expression was scaled within all cells so that their total UMI counts equal the median UMI count of core control cells within the experiment). Each gene was then tested for whether the distribution of normalized expression is identical between control cells bearing non-targeting sgRNAs and cells bearing each perturbation. Only genes detected in at least 3 cells were analyzed, and only cells where at least 200 genes were detected were kept for the analysis. Mann-Whitney U test (scipy.stats.mannwhitneyu) implemented in scipy was used, which tests whether one distribution is stochastically greater than another. The asymptotic P values were used and any perturbation with fewer than 40 cells was excluded. P values were then adjusted for multiple hypothesis testing using the Benjamini-Hochberg and Bonferroni procedure. Gene expression changes that had a corrected P value <=0.05 using either procedure was considered significant.

Data analysis. The library targeted 147 genes, and the downstream analyses focused on 84 genes. Besides perturbations that were eliminated in the quality control steps described above, genes that were either missing in the CRISPRi dataset or whose levels following CRISPRi-mediated knockdown was not <0.33 were excluded. TUBA1C was also excluded as it was observed that one of the designed gRNAs had a perfect match with another gene TUBA1B. For downstream analysis “gene pairs” were examined in which the expression levels of genes (hereafter referred to as observed genes) upon perturbing a given gene (hereafter referred to as perturbed gene) were analyzed. TA-candidate gene-pairs were identified as those where the observed gene was significantly (corrected P value <=0.05) upregulated upon CRISPRn-mediated perturbation of the perturbed gene by a fold change >=1.5 and that were either: a) not significantly upregulated upon CRISPRi-mediated perturbation of the same perturbed gene or, b) if it is, the fold change in upregulation of the observed gene upon CRISPRn-mediate perturbation must be at least 1.5 times higher than what is observed with CRISPRi. Control gene pairs where identified as those with the opposing criteria (i.e., the observed gene is significantly upregulated upon CRISPRi-mediated perturbation of the perturbed gene by a fold change >=1.5 and that was either: a) not significantly upregulated upon CRISPRn-mediated perturbation of the same perturbed gene or, b) if it is, the fold change in upregulation of the observed gene upon CRISPRi-mediate perturbation must be at least 1.5 times higher than what is observed with CRISPRn). This criterion was selected for the control group, to have a control group with the observed (assessed) gene be amenable for upregulation but in a TA-independent manner. This approach allowed for the avoidance of genes in compact heterochromatin environments that aren't amenable to upregulation as controls.

UMAP assessment of similarity in successful perturbation between CRISPRn and CRISPRi responses for each perturbed gene. UMAP was applied to normalized transcriptomic profiles with parameters n_neighbors=2, min_dist=0 and random_state=42 to generate 2-dimensional embeddings for each perturbed gene in either perturb-seq experiments. For each perturbed gene, Euclidean distance in high-dimensional space between the two embeddings were calculated as an imperfect proxy for how similar the transcriptome-wide response between CRISPRn Perturb-seq and CRISPRi Perturb-seq experiments were.

Assessment of CRISPRn and CRISPRi perturb-seq efficiency. CRISPRn and CRISPRi perturbation of a given gene similarly led to transcriptional responses that are a signature of successful perturbations. For each perturbed gene the total number of differentially expressed genes (DEGs) was similar for the two methods of perturbation (FIG. 14C). Moreover, uniform manifold approximation and projection (UMAP) dimension reduction based on observed genes expression levels showed that perturbations clustered based on the identity of the gene and not the method of perturbation (FIG. 14D). Notably, the Euclidean distance in high-dimensional space between transcriptomic profiles upon CRISPRn or CRISPRi perturbation of a given gene did not correlate with the knockdown or NMD levels of the perturbed gene, but was indicated to be influenced by the number of TA-candidate genes (FIGS. 14E and 14F). In addition to these global changes that highlighted similarly successful perturbations of any given genes, there were transcriptional changes that were specific to CRISPRn that represented candidate TA responses, and were annotated as TA-candidate genes. The number of TA-candidate genes for a given perturbation did not correlate with the knockdown level of the gene with CRISPRi, the gene's essentiality, or its initial expression levels in WT cells (FIGS. 14G and 14H).

Annotation of gene elements. Unless noted otherwise, the genetic coordinate information of each gene and its canonical transcript found in Ensembl v109, hg38 was used. The region ±2500 base pair around the transcription start site was annotated as promoter. As there are multiple ways to define enhancers and connect enhancer to genes, annotations from 4 diverse datasets to be comprehensive were used. One of the main ways to define enhancers are from epigenetic marks. Enhancer_epimap are 239,349 candidate-enhancer regions from the Epimap dataset (compbio.mit.edu/epimap/, Boix et al. Nature. 2021 February; 590(7845):300-307). These candidate enhancer elements are defined by the 18-state ChromHMM Roadmap model from observed and imputed tracks of six histone marks (H3K27ac, H3K4me1, H3K4me3, H3K36me3, H3K9me3, H3K27me3) in K562 sample BSS00762. Enhancers were connected to genes using minimum 0.7 correlation threshold between epigenetic marks and gene expression (links_corr_only), as recommended by the authors. Enhancer_ABC are candidate-enhancer regions defined by epigenetic marks for the sample “K562-Roadmap” in Nasser et al. Nature. 2021 May; 593(7858):238-243. Enhancers were connected to gene using prediction from ABC method, which predict enhancer-gene connections based on measurements of chromatin accessibility (ATAC-seq or DNase-seq), histone modifications (H3K27ac ChIP-seq), and chromatin conformation (Hi-C). Enhancer-gene pairs with ABC score >0.015 were used for further analyses, which resulted in 61,981 regions. Enhancer_eRNA_Yulab and Enhancer_eRNA_Lidschreiber are both putative enhancer regions with evidence of transcription of relatively short-lived, divergent enhancer RNA transcripts. Enhancer_eRNA_Yulab are 70,107 proximal and distal elements defined by integrating data from 7 RNA-seq assay methods to detect eRNA in K562 (see e.g., pints.yulab.org; and Yao et al. Nat Biotechnol. 2022 July; 40(7):1056-1065) and linked to the nearest gene. Enhancer_eRNA_Lidschreiber are 12,854 putative enhancer elements that show evidence of intergenic and antisense RNA transcription, identified via transient transcriptome sequencing (see e.g., Lidschreiber et al. Mol Syst Biol. 2021 January; 17(1):e9873). The authors provided 3 methods to connect enhancer to gene (PairedNearest, PairedCorrelatedNeighbouring and PairedCorrelatedWindow). Gene-enhancer pairs linked by at least one method was used.

Sequence similarity analysis. Sequence similarity between the perturbed gene's cDNA sequence and the aforementioned observed gene's elements using BLASTn was performed (see e.g., Altschul et al. J Mol Biol. 1990 Oct. 5; 215(3):403-10). cDNA sequence of the perturbed gene's canonical transcript was obtained from Ensembl v109, along with coordinates of exons, cDNA coding region, and UTRs. Only the observed genes that appeared in TA-candidate and the control gene pairs were included in the BLAST analysis. Genetic coordinates for the observed genes' elements were obtained from the various datasets as mentioned, converted to hg38 coordinates using liftOver if needed, and used to retrieve nucleotide sequence. BLASTn analysis was performed comparing each perturbed gene's cDNA sequence against each of the 6 sequence databases of observed gene's elements (gene body, promoter, enhancer_epimap, enhancer_ABC, enhancer_eRNA_Yulab, enhancer_eRNA_Lidschreiber) with parameters word size 4 and E value up to 100,000. 22,199,045 alignments for 2,135 gene pairs (475 TA-candidate pairs and 1,660 control gene pairs) were obtained, with each gene pair having between 1-6029 alignments. Every gene pair has at least one alignment with E value <1,000.

Epigenetic analyses. Bigwigs of epigenetic marks for K562 sample BSS00762 were downloaded from Epimap (Boix et al. Nature. 2021 February; 590(7845):300-307). The mean value of each gene element defined earlier (gene body, promoter, gene body+promoter) was calculated. Epigenetic signal for each mark between sets of observed genes in different gene-pair categories were compared using nonparametric Wilcoxon test.

ILF3 motif enrichment analysis. Eight motifs for ILF3 in K562 were in mCrossBase, a database of RNA-binding protein binding motifs and crosslink sites defined jointly from ENCODE's eCLIP data (zhanglab.c2b2.columbia.edu/mCrossBase/index.php, Van Nostrand et al. Nature. 2020 July; 583(7818):711-719; and Feng et al. Mol Cell. 2019 Jun. 20; 74(6):1189-1204.e6). MAST from MEME Suite version 5.5.3 was used to search sequences of BLASTn alignments between gene pairs for matches to the set of ILF3 motifs. MAST was specified to score only the exact alignment sequence and not the reverse complement, with threshold Evalue <1,000, and used as background a random sequence model that assumes each position in a random sequence is generated according to the average letter frequencies in the database of all BLASTn alignment sequences. For each sequence, MAST returns a position p-value for each identified motifs, sequence p-value, and sequence E value. High-confidence matches are identified as motif matches with a p-value <0.0001. The sequence p-value is the combined best matches of a sequence to the group of ILF3 motifs, and sequence E value is the probability of observing a sequence p-value at least as small in a random sequence file of the same size. For the 2,135 gene pairs with at least one BLASTn alignment (475 TA-candidate pairs and 1,660 control gene pairs), 341 pairs with at least one high-confidence match to one of eight ILF3 motifs, 1,262 pairs with overall reasonable alignments to ILF3 motifs but no individual high-confidence match, and 532 pairs with no alignments found under the sequence Evalue threshold were identified.

Similarity of genes in Exome-wide association study's significance patterns. Gene-level burden test summary statistics from a recent exome-wide association study, Backman et al. Nature. 2021 November; 599(7886):628-634, was downloaded. The summary statistics was stratified by phenotype, variant consequence (pLOF, DelMissense, pLOF_and_DelMissense) and variant MAF (singleton, <0.001%, <0.01%, <0.1%, <1%). Genes from a) TA candidate gene pairs, b) control gene pairs were included. Each gene a p-value for each combination of [gene]_[variant_consequence]_[MAF]_[phenotype](for example, [ACTG1]_[pLOF]_[<0.001%]_[Coffee_consumed]) was then obtained. This resulted in a matrix of shape (2870, 39,850). PCA is applied to this matrix to reduce it to 2,700 principal components, resulting in a matrix of shape (2870, 2700). The number of components was chosen based on the finding that 2,131 components explain 95% of the variance. Thus, each perturbed gene can be represented by 2,700 numbers. Euclidean distances between gene pairs were calculated from different numbers of consecutive components. Additional experiments were performed with different combinations of components, with similar results observed.

TABLE 1
ILF3 Sequences (Examples 1-3)

| Domain | | | | |
|---|---|---|---|---|
| or | | Mouse | | Human |
| sequence | | SEQ | | SEQ ID |
| name | Mouse Sequence | ID NO | Human Sequence | NO |
| Human | | | MRPMRIFVNDDRHVMAKHSSVYPT | 1 |
| NF110 | | | QEELEAVQNMVSHTERALKAVSD | |
| | | | WIDEQEKGSSEQA | |
| | | | ESDNMDVPPEDDSKEGAGEQKTEH | |
| | | | MTRTLRGVMRVGLVAKGLLLKGD | |
| | | | LDLELVLLCKEKP | |
| | | | TTALLDKVADNLAIQLAAVTEDKY | |
| | | | EILQSVDDAAIVIKNTKEPPLSLTIHL | |
| | | | TSPVVREEM | |
| | | | EKVLAGETLSVNDPPDVLDRQKCL | |
| | | | AALASLRHAKWFQARANGLKSCVI | |
| | | | VIRVLRDLCTRV | |
| | | | PTWGPLRGWPLELLCEKSIGTANRP | |
| | | | MGAGEALRRVLECLASGIVMPDGS | |
| | | | GIYDPCEKEAT | |
| | | | DAIGHLDRQQREDITQSAQHALRLA | |
| | | | AFGQLHKVLGMDPLPSKMPKKPKN | |
| | | | ENPVDYTVQIP | |
| | | | PSTTYAITPMKRPMEEDGEEKSPSK | |
| | | | KKKKIQKKEEKAEPPQAMNALMRL | |
| | | | NQLKPGLQYKL | |
| | | | VSQTGPVHAPIFTMSVEVDGNSFEA | |
| | | | SGPSKKTAKLHVAVKVLQDMGLPT | |
| | | | GAEGRDSSKGE | |
| | | | DSAEETEAKPAVVAPAPVVEAVSTP | |
| | | | SAAFPSDATAENVKQQGPILTKHGK | |
| | | | NPVMELNEKR | |
| | | | RGLKYELISETGGSHDKRFVMEVEV | |
| | | | DGQKFQGAGSNKKVAKAYAALAA | |
| | | | LEKLFPDTPLAL | |
| | | | DANKKKRAPVPVRGGPKFAAKPHN | |
| | | | PGFGMGGPMHNEVPPPPNLRGRGR | |
| | | | GGSIRGRGRGRG | |
| | | | FGGANHGGYMNAGAGYGSYGYGG | |
| | | | NSATAGYSQFYSNGGHSGNASGGG | |
| | | | GGGGGGSSGYGSY | |
| | | | YQGDNYNSPVPPKHAGKKQPHGG | |
| | | | QQKPSYGSGYQSHQGQQQSYNQSP | |
| | | | YSNYGPPQGKQKG | |
| | | | YNHGQGSYSYSNSYNSPGGGGGSD | |
| | | | YNYESKFNYSGSGGRSGGNSYGSG | |
| | | | GASYNPGSHGGY | |
| | | | GGGSGGGSSYQGKQGGYSQSNYNS | |
| | | | PGSGQNYSGPPSSYQSSQGGYGRN | |
| | | | ADHSMNYQYR | |
| Human | | | MRPMRIFVNDDRHVMAKHSSVYPT | 2 |
| NF90de | | | QEELEAVQNMVSHTERALKAVSD | |
| | | | WIDEQEKGSSEQA | |
| | | | ESDNMDVPPEDDSKEGAGEQKTEH | |
| | | | MTRTLRGVMRVGLVAKGLLLKGD | |
| | | | LDLELVLLCKEKP | |
| | | | TTALLDKVADNLAIQLAAVTEDKY | |
| | | | EILQSVDDAAIVIKNTKEPPLSLTIHL | |
| | | | TSPVVREEM | |
| | | | EKVLAGETLSVNDPPDVLDRQKCL | |
| | | | AALASLRHAKWFQARANGLKSCVI | |
| | | | VIRVLRDLCTRV | |
| | | | PTWGPLRGWPLELLCEKSIGTANRP | |
| | | | MGAGEALRRVLECLASGIVMPDGS | |
| | | | GIYDPCEKEAT | |
| | | | DAIGHLDRQQREDITQSAQHALRLA | |
| | | | AFGQLHKVLGMDPLPSKMPKKPKN | |
| | | | ENPVDYTVQIP | |
| | | | PSTTYAITPMKRPMEEDGEEKSPSK | |
| | | | KKKKIQKKEEKAEPPQAMNALMRL | |
| | | | NQLKPGLQYKL | |
| | | | VSQTGPVHAPIFTMSVEVDGNSFEA | |
| | | | SGPSKKTAKLHVAVKVLQDMGLPT | |
| | | | GAEGRDSSKGE | |
| | | | DSAEETEAKPAVVAPAPVVEAVSTP | |
| | | | SAAFPSDATAENVKQQGPILTKHGK | |
| | | | NPVMELNEKR | |
| | | | RGLKYELISETGGSHDKRFVMEVEV | |
| | | | DGQKFQGAGSNKKVAKAYAALAA | |
| | | | LEKLFPDTPLAL | |
| | | | DANKKKRAPVPVRGGPKFAAKPHN | |
| | | | PGFGMGGPMHNEVPPPPNLRGRGR | |
| | | | GGSIRGRGRGRG | |
| | | | FGGANHGGYMNAGAGYGSYGYGG | |
| | | | NSATAGYSDFFTDCYGYHDFGSS | |
| Mouse | MRPMRIFVNDDRHVMAK | 3 | | |
| NF110 | HSSVYPTQEELEAVQNM | | | |
| | VSHTERALKAVSDWIDE | | | |
| | QEKGNSELSEAENMDTPP | | | |
| | DDESKEGAGEQKAEHMT | | | |
| | RTLRGVMRVGLVAKGLL | | | |
| | LKGDLDLELVLLCKEKPT | | | |
| | TALLDKVADNLAIQLTTV | | | |
| | TEDKYEILQSVDDAAIVIK | | | |
| | NTKEPPLSLTIHLTSPVVR | | | |
| | EEMEKVLAGETLSVNDPP | | | |
| | DVLDRQKCLAALASLRH | | | |
| | AKWFQARANGLKSCVIVI | | | |
| | RVLRDLCTRVPTWGPLR | | | |
| | GWPLELLCEKSIGTANRP | | | |
| | MGAGEALRRVLECLASGI | | | |
| | VMPDGSGIYDPCEKEATD | | | |
| | AIGHLDRQQREDITQSAQ | | | |
| | HALRLAAFGQLHKVLGM | | | |
| | DPLPSKMPKKPKNENPVD | | | |
| | YTVQIPPSTTYAITPMKRP | | | |
| | MEEDGEEKSPSKKKKKIQ | | | |
| | KKEEKADPPQAMNALMR | | | |
| | LNQLKPGLQYKLISQTGP | | | |
| | VHAPIFTMSVEVDGSNFE | | | |
| | ASGPSKKTAKLHVAVKV | | | |
| | LQDMGLPTGAEGRDSSK | | | |
| | GEDSAEESDGKPAIVAPP | | | |
| | PVVEAVSNPSSVFPSDAT | | | |
| | TEQGPILTKHGKNPVMEL | | | |
| | NEKRRGLKYELISETGGS | | | |
| | HDKRFVMEVEVDGQKFQ | | | |
| | GAGSNKKVAKAYAALA | | | |
| | ALEKLFPDTPLALEANKK | | | |
| | KRTPVPVRGGPKFAAKPH | | | |
| | NPGFGMGGPMHNEVPPP | | | |
| | PNIRGRGRGGNIRGRGRG | | | |
| | RGFGGANHGGGYMNAG | | | |
| | AGYGSYGYSSNSATAGY | | | |
| | SQFYSNGGHSGNAGGGG | | | |
| | SGGGGGSSSYSSYYQGDS | | | |
| | YNSPVPPKHAGKKPLHG | | | |
| | GQQKASYSSGYQSHQGQ | | | |
| | QQPYNQSQYSSYGTPQG | | | |
| | KQKGYGHGQGSYSSYSN | | | |
| | SYNSPGGGGGSDYSYDS | | | |
| | KFNYSGSGGRSGGNSYGS | | | |
| | SGSSSYNTGSHGGYGTGS | | | |
| | GGSSSYQGKQGGYSSQS | | | |
| | NYSSPGSSQSYSGPASSY | | | |
| | QSSQGGYSRNTEHSMNY | | | |
| | QYR | | | |
| Mouse | MRPMRIFVNDDRHVMAK | 4 | | |
| NF90 | HSSVYPTQEELEAVQNM | | | |
| | VSHTERALKAVSDWIDE | | | |
| | QEKGNSELSEAENMDTPP | | | |
| | DDESKEGAGEQKAEHMT | | | |
| | RTLRGVMRVGLVAKGLL | | | |
| | LKGDLDLELVLLCKEKPT | | | |
| | TALLDKVADNLAIQLTTV | | | |
| | TEDKYEILQSVDDAAIVIK | | | |
| | NTKEPPLSLTIHLTSPVVR | | | |
| | EEMEKVLAGETLSVNDPP | | | |
| | DVLDRQKCLAALASLRH | | | |
| | AKWFQARANGLKSCVIVI | | | |
| | RVLRDLCTRVPTWGPLR | | | |
| | GWPLELLCEKSIGTANRP | | | |
| | MGAGEALRRVLECLASGI | | | |
| | VMPDGSGIYDPCEKEATD | | | |
| | AIGHLDRQQREDITQSAQ | | | |
| | HALRLAAFGQLHKVLGM | | | |
| | DPLPSKMPKKPKNENPVD | | | |
| | YTVQIPPSTTYAITPMKRP | | | |
| | MEEDGEEKSPSKKKKKIQ | | | |
| | KKEEKADPPQAMNALMR | | | |
| | LNQLKPGLQYKLISQTGP | | | |
| | VHAPIFTMSVEVDGSNFE | | | |
| | ASGPSKKTAKLHVAVKV | | | |
| | LQDMGLPTGAEGRDSSK | | | |
| | GEDSAEESDGKPAIVAPP | | | |
| | PVVEAVSNPSSVFPSDAT | | | |
| | TEQGPILTKHGKNPVMEL | | | |
| | NEKRRGLKYELISETGGS | | | |
| | HDKRFVMEVEVDGQKFQ | | | |
| | GAGSNKKVAKAYAALA | | | |
| | ALEKLFPDTPLALEANKK | | | |
| | KRTPVPVRGGPKFAAKPH | | | |
| | NPGFGMGGPMHNEVPPP | | | |
| | PNIRGRGRGGNIRGRGRG | | | |
| | RGFGGANHGGGYMNAG | | | |
| | AGYGSYGYSSNSATAGY | | | |
| | SDFFTDCYGYHDFGAS | | | |
| Mouse | MKRPMEEDGEEKSPSKK | 5 | | |
| NF110 | KKKIQKKEEKADPPQAM | | | |
| deleted | NALMRLNQLKPGLQYKL | | | |
| DZF | ISQTGPVHAPIFTMSVEVD | | | |
| | GSNFEASGPSKKTAKLHV | | | |
| | AVKVLQDMGLPTGAEGR | | | |
| | DSSKGEDSAEESDGKPAI | | | |
| | VAPPPVVEAVSNPSSVFPS | | | |
| | DATTEQGPILTKHGKNPV | | | |
| | MELNEKRRGLKYELISET | | | |
| | GGSHDKRFVMEVEVDGQ | | | |
| | KFQGAGSNKKVAKAYAA | | | |
| | LAALEKLFPDTPLALEAN | | | |
| | KKKRTPVPVRGGPKFAA | | | |
| | KPHNPGFGMGGPMHNEV | | | |
| | PPPPNIRGRGRGGNIRGR | | | |
| | GRGRGFGGANHGGGYM | | | |
| | NAGAGYGSYGYSSNSAT | | | |
| | AGYSQFYSNGGHSGNAG | | | |
| | GGGSGGGGGSSSYSSYY | | | |
| | QGDSYNSPVPPKHAGKK | | | |
| | PLHGGQQKASYSSGYQS | | | |
| | HQGQQQPYNQSQYSSYG | | | |
| | TPQGKQKGYGHGQGSYS | | | |
| | SYSNSYNSPGGGGGSDYS | | | |
| | YDSKFNYSGSGGRSGGNS | | | |
| | YGSSGSSSYNTGSHGGYG | | | |
| | TGSGGSSSYQGKQGGYSS | | | |
| | QSNYSSPGSSQSYSGPASS | | | |
| | YQSSQGGYSRNTEHSMN | | | |
| | YQYR | | | |
| NLS | KRPMEEDGEEKSPSKKK | 6 | KRPMEEDGEEKSPSKKKKKIQKKE | 6 |
| | KKIQKKE | | | |
| NVKQ | | | NVKQ | 7 |
| dsRB1 | NALMRLNQLKPGLQYKL | 8 | NALMRLNQLKPGLQYKLVSQTGPV | 12 |
| | ISQTGPVHAPIFTMSVEVD | | HAPIFTMSVEVDGNSFEASGPSKKT | |
| | GSNFEASGPSKKTAKLHV | | AKLHVAVKVLQDM | |
| | AVKVLQDM | | | |
| DZF | RPMRIFVNDDRHVMAKH | 9 | RPMRIFVNDDRHVMAKHSSVYPTQ | 13 |
| | SSVYPTQEELEAVQNMVS | | EELEAVQNMVSHTERALKAVSDWI | |
| | HTERALKAVSDWIDEQE | | DEQEKGSSEQAESDNMDVPPEDDS | |
| | KGNSELSEAENMDTPPDD | | KEGAGEQKTEHMTRTLRGVMRVG | |
| | ESKEGAGEQKAEHMTRT | | LVAKGLLLKGDLDLELVLLCKEKP | |
| | LRGVMRVGLVAKGLLLK | | TTALLDKVADNLAIQLAAVTEDKY | |
| | GDLDLELVLLCKEKPTTA | | EILQSVDDAAIVIKNTKEPPLSLTIHL | |
| | LLDKVADNLAIQLTTVTE | | TSPVVREEMEKVLAGETLSVNDPPD | |
| | DKYEILQSVDDAAIVIKN | | VLDRQKCLAALASLRHAKWFQAR | |
| | TKEPPLSLTIHLTSPVVRE | | ANGLKSCVIVIRVLRDLCTRVPTWG | |
| | EMEKVLAGETLSVNDPP | | PLRGWPLELLCEKSIGTANRPMGAG | |
| | DVLDRQKCLAALASLRH | | EALRRVLECLASGIVMPDGSGIYDP | |
| | AKWFQARANGLKSCVIVI | | CEKEATDAIGHLDRQQREDITQSAQ | |
| | RVLRDLCTRVPTWGPLR | | HALRLAAFGQLHKVLGMDPLPSKM | |
| | GWPLELLCEKSIGTANRP | | PKKPKNENPVDYTVQIPPSTTYAITP | |
| | MGAGEALRRVLECLASGI | | M | |
| | VMPDGSGIYDPCEKEATD | | | |
| | AIGHLDRQQREDITQSAQ | | | |
| | HALRLAAFGQLHKVLGM | | | |
| | DPLPSKMPKKPKNENPVD | | | |
| | YTVQIPPSTTYAITPM | | | |
| dsRBD2 | NPVMELNEKRRGLKYELI | 10 | NPVMELNEKRRGLKYELISETGGSH | 10 |
| | SETGGSHDKRFVMEVEV | | DKRFVMEVEVDGQKFQGAGSNKK | |
| | DGQKFQGAGSNKKVAKA | | VAKAYAALAALEKL | |
| | YAALAALEKL | | | |
| RGG | RGRGRGGNIRGRGRGRG | 11 | RGRGRGGSIRGRGRGRG | 14 |
| GQSY | SQFYSNGGHSGNAGGGG | 61 | SQFYSNGGHSGNASGGGGGGGGGS | 69 |
| | SGGGGGSSSYSSYYQGDS | | SGYGSY | |
| | YNSPVPPKHAGKKPLHG | | YQGDNYNSPVPPKHAGKKQPHGG | |
| | GQQKASYSSGYQSHQGQ | | QQKPSYGSGYQSHQGQQQSYNQSP | |
| | QQPYNQSQYSSYGTPQG | | YSNYGPPQGKQKG | |
| | KQKGYGHGQGSYSSYSN | | YNHGQGSYSYSNSYNSPGGGGGSD | |
| | SYNSPGGGGGSDYSYDS | | YNYESKFNYSGSGGRSGGNSYGSG | |
| | KFNYSGSGGRSGGNSYGS | | GASYNPGSHGGY | |
| | SGSSSYNTGSHGGYGTGS | | GGGSGGGSSYQGKQGGYSQSNYNS | |
| | GGSSSYQGKQGGYSSQS | | PGSGQNYSGPPSSYQSSQGGYGRN | |
| | NYSSPGSSQSYSGPASSY | | ADHSMNYQYR | |
| | QSSQGGYSRNTEHSMNY | | | |
| | QYR | | | |

TABLE 2
Additional Sequences (Example 1 and Example 3)

| Domain or sequence | | SEQ ID |
|---|---|---|
| name | Amino acid sequence | NO |
| Cas13-ILF3 fusion | MSPKKKRKVEASIEKKKSFAKGMGVKSTLVSGSKVYMTTFAEG | 62 |
| sequence | SDARLEKIVEGDSIRSVNEGEAFSAEMADKNAGYKIGNAKFSHP | |
| | KGYAVVANNPLYTGPVQQDMLGLKETLEKRYFGESADGNDNI | |
| | CIQVIHNILDIEKILAEYITNAAYAVNNISGLDKDIIGFGKFSTVYT | |
| | YDEFKDPEHHRAAFNNNDKLINAIKAQYDEFDNFLDNPRLGYF | |
| | GQAFFSKEGRNYIINYGNECYDILALLSGLAHWVVANNEEESRI | |
| | SRTWLYNLDKNLDNEYISTLNYLYDRITNELTNSFSKNSAANVN | |
| | YIAETLGINPAEFAEQYFRFSIMKEQKNLGFNITKLREVMLDRKD | |
| | MSEIRKNHKVFDSIRTKVYTMMDFVIYRYYIEEDAKVAAANKS | |
| | LPDNEKSLSEKDIFVINLRGSFNDDQKDALYYDEANRIWRKLEN | |
| | IMHNIKEFRGNKTREYKKKDAPRLPRILPAGRDVSAFSKLMYAL | |
| | TMFLDGKEINDLLTTLINKFDNIQSFLKVMPLIGVNAKFVEEYAF | |
| | FKDSAKIADELRLIKSFARMGEPIADARRAMYIDAIRILGTNLSY | |
| | DELKALADTFSLDENGNKLKKGKHGMRNFIINNVISNKRFHYLI | |
| | RYGDPAHLHEIAKNEAVVKFVLGRIADIQKKQGQNGKNQIDRY | |
| | YETCIGKDKGKSVSEKVDALTKIITGMNYDQFDKKRSVIEDTGR | |
| | ENAEREKFKKIISLYLTVIYHILKNIVNINARYVIGFHCVERDAQL | |
| | YKEKGYDINLKKLEEKGFSSVTKLCAGIDETAPDKRKDVEKEM | |
| | AERAKESIDSLESANPKLYANYIKYSDEKKAEEFTRQINREKAKT | |
| | ALNAYLRNTKWNVIIREDLLRIDNKTCTLFANKAVALEVARYV | |
| | HAYINDIAEVNSYFQLYHY | |
| | IMQRIIMNERYEKSSGKVSEYFDAVNDEKKYNDRLLKLLCVPFG | |
| | YCIPRFKNLSIEALFDRNEAAKFDKEKKKVSGNSGSGPKKKRKV | |
| | AAAYPYDVPDYAASGGPSSGAPPPSGGSPAGSPTSTEEGTSESAT | |
| | PESGPGTSTEPSEGSAPGSPAGSPTSTEEGTSTEPSEGSAPGTSTEP | |
| | SEMRPMRIFVNDDRHVMAKHSSVYPTQEELEAVQNMVSHTER | |
| | ALKAVSDWIDEQEKGNSELSEAENMDTPPDDESKEGAGEQKAE | |
| | HMTRTLRGVMRVGLVAKGLLLKGDLDLELVLLCKEKPTTALL | |
| | DKVADNLAIQLTTVTEDKYEILQSVDDAAIVIKNTKEPPLSLTIH | |
| | LTSPVVREEMEKVLAGETLSVNDPPDVLDRQKCLAALASLRHA | |
| | KWFQARANGLKSCVIVIRVLRDLCTRVPTWGPLRGWPLELLCE | |
| | KSIGTANRPMGAGEALRRVLECLASGIVMPDGSGIYDPCEKEAT | |
| | DAIGHLDRQQREDITQSAQHALRLAAFGQLHKVLGMDPLPSKM | |
| | PKKPKNENPVDYTVQIPPSTTYAITPMKRPMEEDGEEKSPSKKK | |
| | KKIQKKEEKADPPQAMNALMRLNQLKPGLQYKLISQTGPVHAP | |
| | IFTMSVEVDGSNFEASGPSKKTAKLHVAVKVLQDMGLPTGAEG | |
| | RDSSKGEDSAEESDGKPAIVAPPPVVEAVSNPSSVFPSDATTEQG | |
| | PILTKHGKNPVMELNEKRRGLKYELISETGGSHDKRFVMEVEV | |
| | DGQKFQGAGSNKKVAKAYAALAALEKLFPDTPLALEANKKKR | |
| | TPVPVRGGPKFAAKPHNPGFGMGGPMHNEVPPPPNIRGRGRGG | |
| | NIRGRGRGRGFGGANHGGGYMNAGAGYGSYGYSSNSATAGYS | |
| | QFYSNGGHSGNAGGGGSGGGGGSSSYSSYY | |
| | QGDSYNSPVPPKHAGKKPLHGGQQKASYSSGYQSHQGQQQPY | |
| | NQSQYSSYGTPQGKQKGYGHGQGSYSSYSNSYNSPGGGGGSDY | |
| | SYDSKFNYSGSGGRSGGNSYGSSGSSSYNTGSHGGYGTGSGGSS | |
| | SYQGKQGGYSSQSNYSSPGSSQSYSGPASSYQSSQGGYSRNTEH | |
| | SMNYQYRASGSGEGRGSLLTCGDVEENPGPVSKGEELFTGVVPI | |
| | LVELDGDVNGHKFSVSGEGEGDATYGKLTLKFICTTGKLPVPW | |
| | PTLVTTLTYGVQCFSRYPDHMKQHDFFKSAMPEGYVQERTIFFK | |
| | DDGNYKTRAEVKFEGDTLVNRIELKGIDFKEDGNILGHKLEYNY | |
| | NSHNVYIMADKQKNGIKVNFKIRHNIEDGSVQLADHYQQNTPIG | |
| | DGPVLLPDNHYLSTQSALSKDPNEKRDHMVLLEFVTAAGITLG | |
| | MDELYK | |
| Mutant Cas13d | IEKKKSFAKGMGVKSTLVSGSKVYMTTFAEGSDARLEKIVEGDS | 63 |
| sequence | IRSVNEGEAFSAEMADKNAGYKIGNAKFSHPKGYAVVANNPLY | |
| | TGPVQQDMLGLKETLEKRYFGESADGNDNICIQVIHNILDIEKIL | |
| | AEYITNAAYAVNNISGLDKDIIGFGKFSTVYTYDEFKDPEHHRA | |
| | AFNNNDKLINAIKAQYDEFDNFLDNPRLGYFGQAFFSKEGRNYII | |
| | NYGNECYDILALLSGLAHWVVANNEEESRISRTWLYNLDKNLD | |
| | NEYISTLNYLYDRITNELTNSFSKNSAANVNYIAETLGINPAEFA | |
| | EQYFRFSIMKEQKNLGFNITKLREVMLDRKDMSEIRKNHKVFDS | |
| | IRTKVYTMMDFVIYRYYIEEDAKVAAANKSLPDNEKSLSEKDIF | |
| | VINLRGSFNDDQKDALYYDEANRIWRKLENIMHNIKEFRGNKT | |
| | REYKKKDAPRLPRILPAGRDVSAFSKLMYALTMFLDGKEINDLL | |
| | TTLINKFDNIQSFLKVMPLIGVNAKFVEEYAFFKDSAKIADELRLI | |
| | KSFARMGEPIADARRAMYIDAIRILGTNLSYDELKALADTFSLDE | |
| | NGNKLKKGKHGMRNFIINNVISNKRFHYLIRYGDPAHLHEIAKN | |
| | EAVVKFVLGRIADIQKKQGQNGKNQIDRYYETCIGKDKGKSVS | |
| | EKVDALTKIITGMNYDQFDKKRSVIEDTGRENAEREKFKKIISLY | |
| | LTVIYHILKNIVNINARYVIGFHCVERDAQLYKEKGYDINLKKLE | |
| | EKGFSSVTKLCAGIDETAPDKRKDVEKEMAERAKESIDSLESAN | |
| | PKLYANYIKYSDEKKAEEFTRQINREKAKTALNAYLRNTKWNV | |
| | IIREDLLRIDNKTCTLFANKAVALEVARYVHAYINDIAEVNSYFQ | |
| | LYHY | |
| | IMQRIIMNERYEKSSGKVSEYFDAVNDEKKYNDRLLKLLCVPFG | |
| | YCIPRFKNLSIEALFDRNEAAKFDKEKKKVSGNS | |
| HEPN domain 1 of | RHWVVH | 64 |
| Cas13Rx | | |
| HEPN domain 2 of | RNKAVH | 65 |
| Cas13Rx | | |
| Mutated HEPN | AHWVVA | 80 |
| domain 1 of Cas13Rx | | |
| Mutated HEPN | ANKAVA | 81 |
| domain 2 of Cas13Rx | | |
| XTEN80 Linker | GGPSSGAPPPSGGSPAGSPTSTEEGTSESATPESGPGTSTEPSEGS | 66 |
| | APGSPAGSPTSTEEGTSTEPSEGSAPGTSTEPSE | |
| Nuclear localization | PKKKRKV | 67 |
| signal | | |
| HA tag | YPYDVPDYA | 68 |

TABLE 3
Trigger RNAs (Example 2)

| Name | Sequence | SEQ ID NO |
|---|---|---|
| 21 | rArUrArArArGrGrArGrArArGrCrUrGrUrGrCrUrArU | 36 |
| 23 | rUrGrArGrCrArArGrArArArUrGrGrCrUrArCrUrGrCrUrG | 37 |
| 24 | rCrGrUrGrArCrArUrArArArGrGrArGrArArGrCrUrGrUrGrC | 38 |
| 27 | rUrGrUrGrCrUrArUrGrUrUrGrCrCrCrUrGrGrArUrUrUrUrGrArGrC | 39 |
| 31 | rArUrGrUrUrGrCrCrCrUrGrGrArUrUrUrUrGrArGrCrArArGrArArArUrGrGrC | 40 |
| 32 | rCrUrGrGrArUrUrUrUrGrArGrCrArArGrArArArUrGrGrCrUrArCrUrGrCrUrGrC | 41 |
| 40 | rArUrArArArGrGrArGrArArGrCrUrGrUrGrCrUrArUrGrUrUrGrCrCrCrUrGrGrArU | 42 |
| | rUrUrUrGrArGrC | |
| 53 | rArUrArArArGrGrArGrArArGrCrUrGrUrGrCrUrArUrGrUrUrGrCrCrCrUrGrGrArU | 43 |
| | rUrUrUrGrArGrCrArArGrArArArUrGrGrCrUrArC | |
| 60 | rUrGrArCrArUrArArArGrGrArGrArArGrCrUrGrUrGrCrUrArUrGrUrUrGrCrCrCrU | 44 |
| | rGrGrArUrUrUrUrGrArGrCrArArGrArArArUrGrGrCrUrArCrUrGrC | |
| 23_mismatch | rCrGrUrGrCrCrCrUrGrCrCrGrGrCrGrCrCrGrCrUrGrUrGrC | 45 |
| (A to C) | | |
| 27_mismatch | rCrGrCrGrCrCrArCrGrCrCrGrCrCrCrCrGrGrArCrCrCrGrGrArGrC | 46 |
| (T to C) | | |
| 31_mismatch | rArCrGrCrCrGrCrCrCrCrGrGrArCrCrCrGrGrArGrCrArArGrArArArCrGrGrC | 47 |
| (T to C) | | |
| 75 | rUrGrUrUrCrGrUrGrArCrArUrArArArGrGrArGrArArGrCrUrGrUrGrCrUrArUrGrU | 87 |
| | rUrGrCrCrCrUrGrGrArUrUrUrUrGrArGrCrArArGrArArArUrGrGrCrUrArCrUrGrC | |
| | rUrGrCrArUrCrArUrC | |

“r” indicates ribonucleotide in Table 3.

TABLE 4
Antisense oligonucleotides (Example 2)

| Name | Sequence | SEQ ID NO |
|---|---|---|
| AP_ctrl | +G*+G*+C*T*A*C*T*A*C*G*C*C*G*+T*+C*+A | 48 |
| AP_1 | +T*+A*+C*G*A*G*A*C*A*T*C*A*A*+G*+G*+A | 49 |
| AP_2 | +C*+C*+A*C*A*G*C*A*G*C*T*T*C*+A*+T*+C | 50 |
| AP_3 | +A*+C*+G*A*G*A*C*A*T*C*A*A*G*+G*+A*+G | 51 |
| AP_4 | +T*+C*+A*A*G*G*A*G*A*A*G*C*T*+G*+T*+G | 52 |
| AP_5 | +G*+G*+A*G*A*A*G*C*T*G*T*G*C*+T*+A*+T | 53 |
| AP_6 | +G*+G*+A*T*T*T*C*G*A*G*A*A*T*+G*+A*+G | 54 |
| AP_7 | +G*+C*+T*G*T*G*C*T*A*T*G*T*A*+G*+C*+C | 55 |
| 2′ | /52MOErC/*/i2MOErC/*/i2MOErT/*/i2MOErA/*/i2MOErT/*A*G*G*A*C*T*A*T*C | 56 |
| MOE_ctrl | *C*/i2MOErA/*/i2MOErG/*/i2MOErG/*/i2MOErA/*/32MOErA/ | |
| 2′ | /52MOErA/*/i2MOErG/*/i2MOErC/*/i2MOErC/*/i2MOErC/*T*G*G*A*T*T*T*C*G | 57 |
| MOE_1 | *A*/i2MOErG/*/i2MOErA/*/i2MOErA/*/i2MOErT/*/32MOErG/ | |
| 2′ | /52MOErT/*/i2MOErG/*/i2MOErT/*/i2MOErA/*/i2MOErC/*G*A*G*A*C*A*T*C*A | 58 |
| MOE_2 | *A*/i2MOErG/*/i2MOErG/*/i2MOErA/*/i2MOErG/*/32MOErA/ | |
| 2′ | /52MOErA/*/i2MOErC/*/i2MOErG/*/i2MOErA/*/i2MOErG/*A*C*A*T*C*A*A*G* | 59 |
| MOE_3 | G*A*/i2MOErG/*/i2MOErA/*/i2MOErA/*/i2MOErG/*/32MOErC/ | |
| 2′ | /52MOErA/*/i2MOErA/*/i2MOErG/*/i2MOErG/*/i2MOErA/*G*A*A*G*C*T*G*T* | 60 |
| MOE_4 | G*C*/i2MOErT/*/i2MOErA/*/i2MOErT/*/i2MOErG/*/32MOErT/ | |

TABLE 5
gRNAs (Example 3)

| Domain | Sequence | SEQ ID NO |
|---|---|---|
| MB 792 Actg2 ex4 antisense RNA Cas13 f | GCAGAGAGAAGATGACCCA | 15 |
| MB 796 Actg2 ex5 antisense RNA Cas13 f | CGCTTCAATGTCCCTGCCA | 16 |
| MB 800 Actg2 ex2 antisense RNA Cas13 f | ACCATGTGTGAAGAAGAGACCAC | 17 |
| MB 798 Actg2 ex6 antisense RNA Cas13 f | AGGCTATTCCTTTGTGACCACAG | 18 |
| MB 810 Actg2 intr1 antisense RNA Cas13 f | AACAAAGAAAGCCAGCCAG | 19 |
| MB 812 Actg2 intr5 antisense RNA Cas13 f | GAAAAATCATTCAGAGCAGACCC | 20 |
| MB 814 Actg2 ex1 sense Cas13 f | CAGAGCAATATACCCCAAG | 21 |
| MB 806 Actg2 ex2 sense Cas13 f | CTGATGTCTAGGGCGGCCCACAA | 22 |
| MB 804 Actg2 sense Cas13 ex3 f | TACTTGAGAGTTAGGATCCCACG | 23 |
| MB 802 Actg2 sense Cas13 ex5 f | AGCAACATACATGGCAGGGACAT | 24 |
| MB 912 Actg2 cas13 intr2 gRNA f | AGTATAGTTCCAATACAGCTCAC | 25 |
| MB 914 Actg2 cas13 intr4 gRNA f | ACTGTCTGAAAAATGCCACAGCA | 26 |
| MB 882 Cdk9 ex6 antisense RNA Cas13 f | GGTAACACAGAGCAGCACCAGCT | 27 |
| MB 884 Cdk9 intr2 antisense RNA Cas13 f | ATAAGTTCAAGGCCACCAT | 28 |
| MB 886 Cdk9 intr6 antisense RNA Cas13 f | CATGAGAAAAAAGCGCCAA | 29 |
| MB 888 Rel ex2 antisense RNA Cas13 f | GTAGAAATAATTGAACAGCCAAG | 30 |
| MB 890 Rel ex8 antisense RNA Cas13 f | ACAAGCTGATGTACACCGCCAAG | 31 |
| MB 876 Sox9 ex1 antisense RNA Cas13 f | TCCAGCAAGAACAAGCCACACGT | 32 |
| MB 878 Sox9 ex2 antisense RNA Cas13 f | AGCACAAGAAAGACCACCCCGAT | 33 |
| MB 880 Sox9 intr1 antisense RNA Cas13 f | AACAATTAGAGGAAGCAAGCCCA | 34 |
| MB 892 Cas13 non targeting gRNA f | TCACCAGAAGCGTACCATACTC | 35 |

TABLE 6
qPCR Primers (Example 3)

| | | SEQ |
|---|---|---|
| | | ID |
| Primer Name | Sequence | NO: |
| MB5 Hprt qpcr f | GATCAGTCAACGGGGGACAT | 70 |
| MB6 Hprt qpcr r | CATTTTGGGGCTGTACTGCTT | 71 |
| MB 525 ACTG2 qPCR f | CCGCCCTAGACATCAGGGT | 72 |
| MB 526 ACTG2 qPCR r | TCTTCTGGTGCTACTCGAAGC | 73 |
| MB 902 mouse Cdk9 qPCR F | TAAAGCCAAGCACCGTCAG | 74 |
| MB 903 mouse Cdk9 qPCR R | GATTTCCCTCAAGGCTGTGAT | 75 |
| MB 904 mouse SOX9 qPCR F | AGACCCTTCGTGGAGGAG | 76 |
| MB 905 mouse SOX9 qPCR R | TCGGTTTTGGGAGTGGTG | 77 |
| MB 898 mouse Rel qPCR f | ATACCTGCCAGATGAAAAAG | 78 |
| MB 899 mouse Rel qPCR r | TCAGTAAAGTGACCACAATC | 79 |

*=Phosphorothioate bonds, MOE=2′-O-methoxyethyl base, +=Affinity Plus (locked nucleic acid base)
5 is 5′ of the oligo, 3 is 3′ and i is internal. 2 refers indeed to a 2′ MOE modification on the nucleotide.

TABLE 7
Non-limiting examples of pairs of perturbed genes and adapting genes

| | Corresponding |
|---|---|
| Perturbed Gene | Adapting Gene |
| ACTG1 | DBR1 |
| ACTG1 | COTL1 |
| ACTG1 | FAAP24 |
| ALDOA | SELENOP |
| ALDOA | TST |
| ALDOA | RTTN |
| ALDOA | FEZ1 |
| ALDOA | RALGPS2 |
| ALDOA | MAN2A2 |
| ALDOA | MYLIP |
| ALDOA | RXRA |
| ALDOA | TXNIP |
| ALDOA | EZH1 |
| ALDOA | FAM20B |
| ALDOA | AHNAK |
| ALDOA | BMPR2 |
| ALDOA | LINC02573 |
| ALDOA | ATP13A2 |
| ALDOA | FCGRT |
| ALDOA | MS4A3 |
| ALDOA | SLC5A3 |
| ALDOA | DHRS1 |
| ALDOA | ASAH1 |
| ALDOA | ZNF714 |
| ALDOA | GATAD2B |
| ALDOA | LETMD1 |
| ALDOA | BEX2 |
| ALDOA | NENF |
| ALDOA | AC079801.1 |
| ANP32B | AC240274.1 |
| ANP32B | MYO9A |
| ANP32B | MGAT2 |
| ANP32B | MMGT1 |
| ANP32B | NME3 |
| ANP32B | UBR2 |
| ANP32B | PSME2 |
| ANXA5 | SHTN1 |
| ANXA5 | RAI14 |
| ANXA5 | FAAP24 |
| ANXA5 | GNPTAB |
| ANXA6 | LINC00339 |
| ANXA6 | ELMSAN1 |
| ANXA6 | SRPRA |
| ARF1 | MIER2 |
| ARF1 | ABHD11 |
| ARF1 | PGAP2 |
| ARF1 | DNM2 |
| ARF1 | ZNF609 |
| ARF1 | RETREG2 |
| ARF1 | WDR26 |
| ARF1 | MAP2K5 |
| ARF4 | CCDC115 |
| ARF5 | AC016629.2 |
| ARF5 | COG7 |
| ARF5 | STK38 |
| ARF5 | LRRC14 |
| ARL6IP5 | USP11 |
| ARL6IP5 | CAMK2G |
| ARL6IP5 | MED20 |
| ARL6IP5 | MRGBP |
| BNIP3 | EIF5A2 |
| BNIP3 | PDE8A |
| BNIP3 | GPRC5D-AS1 |
| BNIP3 | TUBGCP4 |
| BNIP3 | EIF2D |
| BTF3 | SPDYC |
| BTF3 | H19 |
| BTF3 | LRMP |
| BTF3 | TNNI3 |
| BTF3 | PSEN1 |
| BTF3 | SCAMP1-AS1 |
| BTF3 | TM9SF1 |
| BTF3 | AK1 |
| BTF3 | DHRS1 |
| BTF3 | LAPTM4A |
| BTF3 | COG7 |
| BTF3 | DNAJC3-DT |
| BTF3 | HBP1 |
| BTF3 | SH3BGRL |
| BTF3 | TAPBP |
| BTF3 | METRN |
| BTF3 | AKIP1 |
| BTF3 | DST |
| BTF3 | SCNM1 |
| BTF3 | VAMP2 |
| BTF3 | MPC2 |
| BTF3 | SERINC3 |
| BTF3 | LIMD2 |
| BTF3 | S100A13 |
| BTF3 | ZDHHC4 |
| BTF3 | CCDC28B |
| BTF3 | KRT8 |
| BTF3 | CAMTA1 |
| BTF3 | GABPB1-AS1 |
| BTF3 | LGALS1 |
| BTF3 | ABO |
| BTF3 | ATP6V0A1 |
| BTF3 | S100A11 |
| BTF3 | PRR13 |
| BTF3 | NELFE |
| BTF3 | WASF2 |
| BTF3L4 | AC003093.1 |
| BTF3L4 | TMEM87B |
| BTF3L4 | TP5313 |
| BTF3L4 | Z94721.3 |
| BTF3LA | PLEKHH3 |
| BTF3L4 | PRIM2 |
| BTF3LA | STK19 |
| BTF3L4 | ACYP1 |
| BTF3L4 | HAGH |
| CAPZA2 | SP2 |
| CAPZA2 | SRPRA |
| CAPZA2 | CNOT11 |
| CAPZA2 | CUEDC2 |
| CARHSP1 | POLR1A |
| CARHSP1 | KBTBD6 |
| CBX1 | TRPM7 |
| CBX1 | GOLT1B |
| CBX1 | GDAP2 |
| CCND2 | SLC7A11 |
| CCND2 | ABO |
| CCND2 | ARL11 |
| CCND2 | TRIP11 |
| CCND2 | PFDN1 |
| CCND2 | PDCD10 |
| CCND3 | MPRIP |
| CCND3 | HBA1 |
| CCND3 | SLC30A1 |
| CCND3 | REEP6 |
| CCND3 | SMIM1 |
| CCND3 | LGR4 |
| CCND3 | MOB3B |
| CCND3 | CIT |
| CCND3 | IREB2 |
| CCND3 | MYLIP |
| CCND3 | SLC39A8 |
| CCND3 | ABCB7 |
| CCND3 | PARP4 |
| CCND3 | AL445524.1 |
| CCND3 | NPRL3 |
| CCND3 | AASDH |
| CCND3 | TRMT11 |
| CCND3 | GNPTAB |
| CCND3 | UBXN7 |
| CLTC | SLC6A8 |
| CLTC | LRP12 |
| CLTC | ARHGDIG |
| CLTC | RBL2 |
| CLTC | ALDOC |
| CLTC | MGAT3 |
| CLTC | TUBB2B |
| CLTC | AP001531.1 |
| CLTC | PSEN1 |
| CLTC | TSPAN32 |
| CLTC | SGTB |
| CLTC | TMEM54 |
| CLTC | WNK4 |
| CLTC | CSF3R |
| CLTC | MYRF |
| CLTC | TFRC |
| CLTC | ARL4A |
| CLTC | AAK1 |
| CLTC | BSDC1 |
| CLTC | HES4 |
| CLTC | SLC37A1 |
| CLTC | ABCC4 |
| CLTC | TAPBP |
| CLTC | ADAMTSLA |
| CLTC | AC079466.1 |
| CLTC | AC025171.1 |
| CLTC | TET2 |
| CLTC | FOXO3 |
| CLTC | SERPINF1 |
| CLTC | EGLN3 |
| CLTC | AIP |
| CLTC | CNST |
| CLTC | ISYNA1 |
| CLTC | CTSD |
| CLTC | ANKRD10 |
| CLTC | LETMD1 |
| CLTC | ZNF292 |
| CLTC | ACSM3 |
| CLTC | LMNA |
| CLTC | PLD3 |
| CREB3 | THAP6 |
| CREB3 | ZNF720 |
| CREB3 | RAB5A |
| CSNK1E | ELP3 |
| CSNK1E | ZEB1 |
| DDX21 | AC003093.1 |
| DDX21 | POLR3C |
| DDX21 | MAN2A2 |
| DDX21 | DDX59 |
| DDX21 | KCMF1 |
| DDX21 | TAC3 |
| DDX21 | RGS16 |
| DDX21 | LINC02573 |
| DDX21 | ARMCX6 |
| DDX21 | PARP12 |
| DDX21 | RAB32 |
| DDX21 | VAMP2 |
| DDX21 | ARFGAP3 |
| DDX21 | SEC11C |
| DDX21 | ENTPD6 |
| DDX21 | C8orf82 |
| DDX21 | TSC22D1 |
| DDX21 | SOX12 |
| DDX21 | C1orf35 |
| DDX21 | TATDN2 |
| DDX21 | GRINA |
| DPYSL2 | FAM219B |
| EIF4A1 | AC240274.1 |
| EIF4A1 | RASA1 |
| EIF4A1 | SLC30A1 |
| EIF4A1 | MANBA |
| EIF4A1 | ZIC2 |
| EIF4A1 | POMGNT1 |
| EIF4A1 | USB1 |
| EIF4A1 | TIAL1 |
| EIF4A1 | SNHG7 |
| EIF4A2 | SKIV2L |
| EIF4A2 | CLEC2L |
| EIF4A2 | KIT |
| EIF4A2 | METTL8 |
| EIF4E | PLAA |
| EIF4E | MANBA |
| EIF4E | PIGQ |
| EIF4E | INTS4 |
| EIF4E | C11orf68 |
| EIF4E | HAUS3 |
| EIF4E | LRFN4 |
| EIF4E | PTDSS2 |
| EIF4E | FBXL6 |
| EIF4E | INTS12 |
| EIF4E | KLHL21 |
| EIF4E | HELQ |
| EIF4E | PPFIA3 |
| EIF4E | CDS2 |
| EIF4E | TXLNG |
| EIF4E | EFL1 |
| EIF4E | CCDC86 |
| EIF4E | RIPOR3 |
| EIF4E | PXK |
| EIF4E | PAPSS1 |
| EIF4E | SH3GLB2 |
| EIF4E | CAMK2G |
| EIF4E | IQCH-AS1 |
| EIF4E | UBA3 |
| EIF4E | PACSIN3 |
| EIF4E | SHARPIN |
| EIF4E | MIF4GD |
| EIF4E | PIK3CG |
| EIF4E | RRAGD |
| EIF4E | VPS16 |
| EIF4E | MT1F |
| EIF4E | ENTPD6 |
| EIF4E | CYP20A1 |
| EIF4E | HSF |
| EIF4E | TPGS1 |
| EIF4E | MFSD12 |
| EIF4E | EIPR1 |
| EIF4E | HOOK2 |
| EIF4E | CD151 |
| EIF4E | GUSB |
| EIF4E | RGS16 |
| EIF4E | DEDD |
| EIF4E | YY1AP1 |
| EIF4E | ANKZF1 |
| EIF4E | C11orf1 |
| EIF4E | VRK1 |
| EIF4E | ZFAND2B |
| EIF4E | NIFK-AS1 |
| EIF4E | RFC5 |
| EIF4E | EPAS1 |
| EIF4E | WDR74 |
| EIF4E | ATPAF1 |
| EIF4E | DUXAP8 |
| EIF4E | SERPINF1 |
| EIF4E | UBE2Q1 |
| EIF4E | SLC30A9 |
| EIF4E | CMSS1 |
| EIF4E | TAF9 |
| EIF4E | TMEM60 |
| EIF4E | GATAD1 |
| EIF4E | TRABD |
| EIF4E | PFKL |
| EIF4E | OS9 |
| EIF4E | BMPR2 |
| EIF4E | HIST1H2AG |
| EIF4E | NQO1 |
| EIF4E | TAOK1 |
| FERMT2 | HYLS1 |
| FERMT2 | SLC30A1 |
| FERMT2 | CCDC92 |
| FTL | OTUD3 |
| FTL | MMGT1 |
| FTL | CULAB |
| FTL | LAMTOR3 |
| FTL | DCTN3 |
| GAPDH | ZNF117 |
| GAPDH | TSC22D1 |
| GAPDH | LINC02573 |
| GAPDH | LTN1 |
| GAPDH | ACSM3 |
| GAPDH | ZEB2 |
| GAPDH | SERPINF1 |
| GNL3 | ANKRD33B |
| GNL3 | MINCR |
| GNL3 | WASHC3 |
| GNL3 | TFAP2B |
| GNL3 | AC139493.2 |
| GNL3 | IFRD1 |
| GNL3 | AC003093.1 |
| GNL3 | PACC1 |
| GNL3 | HEIH |
| GNL3 | RFESD |
| GNL3 | BPGM |
| GNL3 | IQCH-AS1 |
| GNL3 | MAP2K5 |
| GNL3 | EMILIN2 |
| GNL3 | VAT1 |
| GNL3 | PXN-AS1 |
| GNL3 | SMG9 |
| GNL3 | ZNF433-AS1 |
| GNL3 | FAM92A |
| GNL3 | OBSCN |
| GNL3 | ERI2 |
| GNL3 | EXOSC1 |
| GNL3 | ARFGAP3 |
| GNL3 | ARMCX6 |
| GNL3 | XPA |
| GNL3 | ATP6V1C1 |
| GNL3 | AC246817.2 |
| GNL3 | AGPAT4 |
| GNL3 | DUSP3 |
| GNL3 | CLEC2L |
| GNL3 | AKAP13 |
| GNL3 | SMIM1 |
| GNL3 | CMC4 |
| GNL3 | TAMM41 |
| GNL3 | ZCCHC10 |
| GNL3 | AC022075.1 |
| GNL3 | YBEY |
| GNL3 | PPP2R3C |
| GNL3 | HBZ |
| GNL3 | ERMAP |
| GNL3 | DLEU1 |
| GNL3 | SNHG7 |
| GNL3 | PAFAH1B1 |
| GNL3 | TRMT1 |
| GNL3 | MRPS10 |
| GNL3 | ZNRF1 |
| GNL3 | BANP |
| GNL3 | TBCC |
| GNL3 | PPP1R15A |
| GNL3 | SNHG17 |
| GNL3 | METTL23 |
| GNL3 | PDRG1 |
| GNL3 | PITHD1 |
| GNL3 | MRPS18C |
| GNL3 | JUN |
| GNL3 | TMEM126A |
| GNL3 | NOSIP |
| GNL3 | THAP11 |
| GNL3 | HOMER3 |
| H2AFV | AC015813.1 |
| H2AFV | ZNF827 |
| H2AFV | OSTF1 |
| H2AFV | ADAM17 |
| H2AFZ | PFAS |
| H2AFZ | EVA1B |
| H2AFZ | ABHD16A |
| HIF1A | TGFBRAP1 |
| HIF1A | GGA2 |
| HIF1A | FAAP24 |
| HIF1A | MAGEA12 |
| HIF1A | MAP2K5 |
| HMGB2 | SLC9B2 |
| HMGB2 | COPG1 |
| HMGB2 | SUPT3H |
| HMGB2 | RCSD1 |
| HMGB2 | AKAP17A |
| HMGB2 | ISYNA1 |
| HMGB2 | XRCC2 |
| HMGB2 | SMU1 |
| HNRNPA1 | AC016629.2 |
| HNRNPA1 | AP002360.1 |
| HNRNPA1 | DOCK11 |
| HNRNPA1 | SLC25A19 |
| HNRNPA1 | FAM122B |
| HNRNPA1 | PRPF18 |
| HNRNPA1 | FAHD1 |
| HNRNPA1 | WASHC1 |
| HNRNPA1 | BCR |
| HNRNPA1 | ALAS1 |
| HNRNPA1 | GLTP |
| HNRNPA1 | DPM3 |
| HNRNPA1 | MAP3K2 |
| HNRNPA1 | TTK |
| HNRNPA1 | ANKRD39 |
| HNRNPA1 | STXBP6 |
| HNRNPA1 | STRN4 |
| HNRNPA1 | GGA2 |
| HNRNPA1 | EGLN3 |
| HNRNPA1 | MRM3 |
| HNRNPA1 | KBTBD6 |
| HNRNPA1 | FBXO17 |
| HNRNPA1 | TMEM158 |
| ID1 | EPAS1 |
| ID1 | PDE8A |
| KPNA1 | PLEKHO1 |
| KPNA1 | EXOC6 |
| MAPT | TNRC18 |
| MAPT | ALOX12-AS1 |
| MAPT | BCR |
| MAPT | MEF2C |
| MAPT | KBTBD6 |
| MAPT | MFSD3 |
| MELK | PACC1 |
| MELK | SLC25A15 |
| MELK | THAP6 |
| MELK | SPAG5 |
| MELK | ULK3 |
| MELK | DHRS1 |
| MELK | MAP2K5 |
| MELK | OXCT1 |
| MELK | ZDHHC6 |
| MELK | PSEN1 |
| MELK | NUP98 |
| NAA10 | NAPRT |
| NME3 | TICRR |
| NME3 | RGP1 |
| NME3 | VARS2 |
| NME3 | HDHD2 |
| NME3 | HAUS6 |
| NME3 | PIGF |
| NME3 | NEFH |
| NME3 | TSTD2 |
| NME4 | SLC30A1 |
| NME4 | MON1A |
| NME4 | SFXN5 |
| NME4 | WDR48 |
| NME4 | TRIP11 |
| NME4 | MAN1A1 |
| NONO | EIF5A2 |
| NONO | ERF |
| NONO | EGLN3 |
| NONO | FEZ2 |
| NUCB2 | MCM9 |
| NUCB2 | EIF5A2 |
| PABPN1 | AP002387.2 |
| PGAM1 | TMOD1 |
| PGAM1 | MXD1 |
| PGAM1 | EPAS1 |
| PGAM1 | NUCB2 |
| PGAM1 | TNRC6B |
| PGK1 | IGFL2-AS1 |
| PGK1 | MAP2K5 |
| PGK1 | ZSCAN16-AS1 |
| PGK1 | KCNQ1OT1 |
| PGK1 | GALM |
| PKM | HBD |
| PKM | HEMGN |
| PKM | ADAMTSLA |
| PKM | ANXA2R |
| PKM | VEZF1 |
| PKM | ZNF431 |
| PKM | TSC22D1 |
| PKM | NPRL3 |
| PKM | ZNF775 |
| PKM | ETHE1 |
| PKM | PKIG |
| PKM | GYPE |
| PKM | AC079466.1 |
| PPA1 | MAPT |
| PPIG | SUPV3L1 |
| PPIG | VTI1B |
| PRDX1 | EML3 |
| PRDX1 | PLEKHO1 |
| PRDX1 | NRSN2 |
| PRDX1 | NAB1 |
| PRDX1 | ABO |
| PRDX1 | ENTPD6 |
| PRDX1 | HOXB2 |
| PRDX1 | OSTF1 |
| PRDX1 | THAP12 |
| PRDX1 | LCOR |
| PRDX1 | CLIP1 |
| PRDX1 | FAAP24 |
| PRDX1 | ARHGEF1 |
| PRDX1 | SYTLA |
| PRDX1 | OGT |
| PRDX1 | MDK |
| PRDX1 | STARD3NL |
| PRDX1 | TMEM192 |
| PRDX1 | UBE2L6 |
| PRDX1 | PCSK7 |
| PRDX1 | TRAPPC6A |
| PRDX1 | RAB27A |
| PRDX2 | OSBPL2 |
| PRDX2 | VPS37B |
| PRPF40A | CLCA1 |
| PRPF40A | ATF7IP2 |
| PRPF40A | GSN |
| PRPF40A | TUBA1A |
| PRPF40A | YPEL3 |
| PRPF40A | C2orf27A |
| PRPF40A | DPYSL2 |
| PRPF40A | KLHL24 |
| PRPF40A | S100A13 |
| PRPF40A | LINC02327 |
| PRPF40A | TMEM168 |
| PRPF40A | ZNF736 |
| PRPF40A | COG3 |
| PRPF40A | HBP1 |
| PRPF40A | NES |
| PRPF40A | MMP24OS |
| PRPF40A | MAP4K3 |
| PRPF40A | LCP2 |
| PRPF40A | SKIL |
| PRPF40A | RBM48 |
| PRPF40A | PLEKHO1 |
| PRPF40A | IER3 |
| PRPF40A | PSEN1 |
| PRPF40A | FCER1G |
| PRPF40A | DYRKIA |
| PRPF40A | CYTH1 |
| PRPF40A | TUBB2A |
| PRPF40A | CLN5 |
| PRPF40A | YPEL5 |
| PRPF40A | TRPM4 |
| PRPF40A | PKNOX1 |
| PRPF40A | HLCS |
| PRPF40A | TAF8 |
| PRPF40A | CLN8 |
| PRPF40A | AFTPH |
| PRPF40A | TRIM38 |
| PRPF40A | TRAPPC2B |
| PRPF40A | JUN |
| PRPF40A | AAK1 |
| PRPF40A | LINC00342 |
| PRPF40A | MANBA |
| PRPF40A | HDAC8 |
| PRPF40A | CALMLA |
| PRPF40A | GYPE |
| PRPF40A | COMMD7 |
| PRPF40A | ARID5B |
| PRPF40A | MOCS2 |
| PRPF40A | PARP14 |
| PRPF40A | ARMCX3 |
| PRPF40A | SELENOP |
| PRPF40A | RCBTB1 |
| PRPF40A | NEU1 |
| PRPF40A | PIK3CG |
| PRPF40A | AP1S2 |
| PRPF40A | RPAP2 |
| PRPF40A | GOLGA4 |
| PRPF40A | PIM1 |
| PRPF40A | ITSN1 |
| PRPF40A | CHCHD6 |
| PRPF40A | ZNF292 |
| PRPF40A | IL6ST |
| PRPF40A | DBI |
| PRPF40A | UBE2H |
| PRPF40A | TOPORS |
| PRPF40A | SERINC1 |
| PRPF40A | MKLN1 |
| PRPF40A | FNIP2 |
| PRPF40A | VEZF1 |
| PRPF40A | ARL6IP5 |
| PRPF40A | CCDC107 |
| PRPF40A | ARF3 |
| PRPF40A | WDR48 |
| PRPF40A | FAM204A |
| PRPF40A | BHLHE40 |
| PRPF40A | TPM1 |
| PRPF40A | PXN |
| PRPF40A | ITM2B |
| PRPF40A | SSBP2 |
| PRPF40A | LINC00667 |
| PRPF40A | SNRNP48 |
| PRPF40A | GCC2 |
| PRPF40A | PLCL2 |
| PRPF40A | TMF1 |
| PRPF40A | RICTOR |
| PRPF40A | CALCOCO2 |
| PRPF40A | BMPR2 |
| PRPF40A | TAF9 |
| PRPF40A | ARFGAP3 |
| PRPF40A | MPC2 |
| PRPF40A | TAX1BP1 |
| PRPF40A | GLTP |
| PRPF40A | PURB |
| PRPF40A | CLU |
| PRPF40A | LAPTM4A |
| PRPF40A | BACH1 |
| PRPF40A | TATDN2 |
| PRPF40A | PHC3 |
| PRPF40A | AKAP8L |
| PRPF40A | AK1 |
| PRPF40A | KDM5A |
| PRPF40A | SOCS2 |
| PRPF40A | LAPTM5 |
| PRPF40A | ERCC1 |
| PRPF40A | MIS18BP1 |
| PRPF40A | TEN1 |
| PRPF40A | KRT8 |
| PRPF40A | SEC62 |
| PRPF40A | TMEM106C |
| PRPF40A | ANKRD11 |
| PRPF40A | CKAP2 |
| PRPF40A | GATAD2B |
| PRPF40A | ARL6IP1 |
| PRPF40A | TMEM219 |
| PRPF40A | CCDC88A |
| PRPF40A | KDM5B |
| PRPF40A | CCNG1 |
| PRPF40A | FAM120AOS |
| PRPF40A | RAB14 |
| PRPF40A | RALA |
| PRPF40A | NIPBL |
| PRPF40A | GAS5 |
| PRPF40A | SLC3A2 |
| PRPF40A | PGLS |
| RB1 | ARL8A |
| RB1 | CPT2 |
| REL | CHTF18 |
| REL | ARHGEF39 |
| REL | RAB18 |
| REL | MAP2K5 |
| REL | NIFK-AS1 |
| REL | ZNF787 |
| REL | MED24 |
| RELA | MGAT2 |
| RPL22 | WDR35 |
| RPL22 | POC5 |
| RPL22 | RHNO1 |
| RPL22 | PRMT6 |
| RPL22 | TDP1 |
| RPL22 | RHOB |
| RPL22 | SAP30L |
| RPL22 | ARHGEF39 |
| RPL22 | PIGL |
| RPL22 | PIF1 |
| RPL22 | TRIP13 |
| RPL22 | PDLIM5 |
| RPL22 | MAP1S |
| RPL22 | TRIM71 |
| RPL22 | RYBP |
| RPL22 | LYRM1 |
| RPL22 | RMND5A |
| RPL22 | GPAA1 |
| RPL22 | PSMA3-AS1 |
| RPL22 | NBR2 |
| RPL22L1 | RPL22L1 |
| RPL22L1 | TRIM71 |
| RPL26 | SERTADI |
| RPL26 | TAC3 |
| RPL26 | SNHG11 |
| RPL26 | TSC22D1 |
| RPL26 | RNF166 |
| RPL26 | ALAS1 |
| RPL26 | LRIF1 |
| RPL26 | ZNF720 |
| RPL26 | THAP11 |
| RPL26 | ARMCX6 |
| RPL26 | TMEM161B |
| RPL26L1 | PMS2 |
| RPL26L1 | RNASE1 |
| RPL36AL | ZNF589 |
| RPL36AL | NEK4 |
| RPL36AL | ALAS2 |
| RPL36AL | CMTM4 |
| RPL36AL | HIVEP3 |
| RPL36AL | UBAP1 |
| RPL36AL | PRMT6 |
| RPL36AL | CCSER2 |
| RPL36AL | TMEM54 |
| RPL36AL | ETV6 |
| RPL36AL | TMEM94 |
| RPL36AL | LGALSL |
| RPL36AL | TMEM161B |
| RPL36AL | YIPF6 |
| RPL36AL | ZSCAN30 |
| SFT2D1 | JMY |
| SFT2D1 | TBCK |
| SFT2D1 | ELP3 |
| SIRT1 | C6orf52 |
| SLC25A6 | TRIM25 |
| STK25 | BRMS1L |
| STK25 | YAE1 |
| STK25 | ZNF611 |
| STK25 | GNPDA2 |
| STK25 | GLTP |
| STK25 | MAGEC1 |
| STK25 | MFSD1 |
| STK25 | API5 |
| STK25 | SEC23B |
| STK25 | ZNF280D |
| STK25 | AGPAT4 |
| TAF7 | HIST1H1C |
| TAF7 | AC016074.2 |
| TAF7 | HIST1H4H |
| TAF7 | WAC-AS1 |
| TAF7 | NQO1 |
| TAF7 | MKNK2 |
| TAF7 | POLR2A |
| TAF7 | BRD2 |
| TAF7 | SQSTM1 |
| TAF7 | S100A13 |
| TAF7 | TAFA2 |
| TAF7 | MDP1 |
| TAF7 | FTL |
| TAF7 | HIST1H2BC |
| TAF7 | CPT2 |
| TAF7 | SURF1 |
| TAF7 | TRAPPC6A |
| TAF7 | HIST1H2BJ |
| TAF7 | UROS |
| TAF7 | RRM2 |
| TAF7 | DBF4B |
| TAF7 | MFSD3 |
| TAF7 | TXNIP |
| TAF7 | RELN |
| TAF7 | ENDOG |
| TAF7 | HOXB-AS1 |
| TAF7 | COPS6 |
| TAF7 | TTC17 |
| TAF7 | HIST1H2AG |
| TAF7 | ACSM3 |
| TET: | ZNF213-AS1 |
| TET1 | MAP3K3 |
| TET1 | CASC4 |
| TET1 | C8orf82 |
| TMUB1 | MAP3K2 |
| TUBA1B | UXS1 |
| TUBA1B | TRAK1 |
| TUBA1B | RNF144A-AS1 |
| TUBA1B | CLCN5 |
| TUBA1B | AP001531.1 |
| TUBA1B | CDK5RAP3 |
| TUBA1B | ZFP36L2 |
| TUBA1B | FAM210B |
| VCL | UBE2H |
| VCL | FAAP24 |
| VDAC2 | TRDMT1 |
| VDAC2 | RNF113A |
| VDAC2 | TP53BP1 |
| VDAC3 | TMEM43 |
| VDAC3 | GPR108 |
| VDAC3 | AP1S2 |
| ZFAND5 | ZNF720 |
| ZFAND5 | ACYP1 |
| ZFAND5 | URB1 |

TABLE 8
Non-limiting examples of corresponding adapting genes for ACTG1 is
as the perturbed gene.

| | | | | | |
|---|---|---|---|---|---|
| Ptpn18 | Rhbdf2 | Dennd1c | Prkcz | Sall1 | Gal3st1 |
| 2010300C02Rik | Nptx1 | Tmem178 | Ttll10 | Irf8 | Wdfy4 |
| Fzd6 | Aatk | Dsg2 | Prkag2os2 | Cbfa2t3 | Olfr1372-ps1 |
| Pou3f3 | Fam49a | Mocos | Prkag2os1 | Igsf9b | 4930519K11Rik |
| Sox13 | Rnf144a | Nrg2 | 2900005J15Rik | St14 | Pde1b |
| Elf3 | Slc25a21 | Gm9926 | Gm16401 | Barx2 | Rbm47 |
| Lad1 | 4930512B01Rik | 9030625G05Rik | Gm43605 | Tlcd5 | Alkbh2 |
| Igfn1 | Sptb | Myrf | Arap2 | St6galnac3 | Gm47218 |
| Fmo1 | Papln | Pip5k1b | Gm43281 | Slc35f2 | Mep1a |
| Fmo2 | A630072L19Rik | Tmem252 | Atp8a1 | Gm2735 | Gm39090 |
| Vangl2 | Serpina10 | Pyroxd2 | Sowahb | Car12 | Itgb4 |
| Susd4 | Slc17a1 | Cacna1b | Hpse | Aldh1a2 | Gm12715 |
| C130074G19Rik | Dsp | Kcnt1 | Cds1 | Wdr72 | Gsdmd |
| Gm26674 | H3c11 | Gca | Gm43273 | Fam83b | Fjx1 |
| Arfgef3 | Dapk1 | Kcnh7 | Mfsd7a | Gm47430 | Mrp135 |
| Soga3 | Slc6a18 | B3galt1 | Glt1d1 | Gm16010 | Egr2 |
| H2ac13 | Fam169a | Large2 | Mlxipl | Nmnat3 | Mrps21 |
| C730027H18Rik | Ocln | Spint1 | Adap1 | Slc38a3 | Samd10 |
| Icosl | A630072M18Rik | Ltk | Gm26814 | Dclk3 | Ppm1j |
| Amdhd1 | Ripk3 | Gpat2 | Ica1 | Scn5a | Lamb3 |
| Tbc1d30 | Slain1os | Zcchc3 | Kcp | Lonrf3 | Vwa7 |
| Rnf130 | Tspy15 | Eya2 | Aoc1 | Prrg1 | Sh3bgrl2 |
| Acs16 | Tex15 | Atp9a | Chn2 | Cldn2 | Gm31683 |
| Slc47a1 | Gm32025 | Tshz2 | 9130019P16Rik | 9130017K11Rik | Pex11b |
| Fam83g | AU022754 | P2ry13 | Ghrhr | Cd300a | Mapk4 |
| Dnah2 | Shank3 | Pklr | Tgfa | St6galnac2 | Pctp |
| Ybx2 | Dhh | Tchh | Il17re | Chdh | Sh3d21 |
| Slc16a11 | Smagp | Ddit41 | Mkrn2os | Celsr1 | Aldh3a1 |
| Slc16a13 | Scn8a | A530083M17Rik | St8sia1 | Pgm5 | Lrrc7 |
| Smtnl2 | Fignl2 | Gm43486 | Usp29 | D630003M21Rik | Nsa2 |
| Doc2b | Hunk | Esrp1 | Nfkbid | A730020M07Rik | Dlec1 |
| Slc13a2 | Igsf5 | Ccdc96 | Svip | Arhgef16 | Ryr1 |
| Ttll6 | Ripk4 | Galnt12 | Ucp2 | Bend4 | Trim68 |
| Plxdc1 | Caskin1 | Ambp | Rassf10 | Nrip2 | Gm37522 |
| Grb7 | Gm50269 | Dmbx1 | Adgra1 | Iqgap2 | Syt14 |
| C1ql1 | B4galnt3 | Grhl3 | Ano9 | Gm49326 | Gm38059 |
| Cacng4 | Ttbk1 | Tmem51 | Spaca7 | B4galnt1 | Dock8 |
| Sdk2 | AI661453 | Kazn | 5830408C22Rik | Dock3 | Gm38245 |
| Kif19a | Sult1c2 | Gpr157 | Fcho1 | Ar | Afap1l1 |
| Fads6 | Crb3 | Espn | Rtbdn | Thsd4 | Sh3tc1 |
| Pdlim4 | Map7 | Ntm | Wasf3 | | |
| Arhgap27 | Plxnb1 | Pkhd1 | Map3k5 | | |
| Gch1 | Slc1a2 | Rp137-ps1 | Hoxc4 | | |
| Parp12 | Cd1d1 | Atf3 | Septin3 | | |
| Ehf | Tfcp2l1 | Hoxb2 | Gng7 | | |
| Misp3 | Cytip | Adora1 | Hoxc9 | | |
| Gm16574 | Aldh1l1 | Hoga1 | P2ry14 | | |
| Ccdc40 | Prkn | Cdk18 | Ggta1 | | |
| Abcc3 | Gm8378 | Chd7 | Eif4g3 | | |
| Lhx1 | Chn1 | Rasgef1b | Cap2 | | |
| Fras1 | Cadm1 | Tspoap1 | Ypel2 | | |
| Pmaip1 | 2810429I04Rik | Gfra1 | Mtcl1 | | |
| Miga1 | Slc9a2 | Fam167a | Epha4 | | |
| Fgd4 | Corin | Trim14 | Mgat4a | | |
| Celsr2 | Pitx2 | Phactr2 | Myb | | |
| Tnnt2 | Pkp1 | Gstt3 | Rnf157 | | |
| Tm6sf1 | Foxal | Ralgps2 | Limch1 | | |
| Fry | Tmem231 | Ankrd33b | C130089K02Rik | | |
| Esrrg | Syt7 | Cttnbp2 | H2ac20 | | |
| Cdkl1 | Rundc3a | Ntrk3 | Sdk1 | | |
| Mctp2 | Trmt9b | Slco1a6 | Adamts9 | | |
| Hoxc5 | Dclk2 | Lama1 | Csmd3 | | |
| Plekha2 | Shisa7 | Tnik | | | |
| Mdga1 | Bmp6 | Nup210 | | | |
| Ppp1r26 | Proser2 | Thbs2 | | | |
| Hdgf13 | Hoxb7 | Blnk | | | |
| Rnf180 | Cxxc5 | Lyn | | | |
| Ifnlr1 | Ttc6 | Eml5 | | | |
| Pax9 | Gimap9 | Scamp5 | | | |
| Atp6v0e2 | Frmd4b | Zfp612 | | | |
| Irx3os | Arhgap6 | Stard8 | | | |
| Gm6211 | Rph3al | Pacsin1 | | | |
| Eeflakmt3 | Fam221a | Nova1 | | | |
| Zbed5 | Adamts16 | Ccdc88c | | | |
| Iglon5 | Tspan33 | Dennd2a | | | |
| Flt4 | Parm1 | Inava | | | |
| Slc13a2os | Actg2 | Stox2 | | | |
| Nr1h4 | Ap1g2 | Hoxb8 | | | |
| Exph5 | Igsf11 | Megf6 | | | |
| Pde3b | Rasl12 | Dpysl5 | | | |

### REFERENCES

1. Wessels H H, Méndez-Mancilla A, Guo X, Legut M, Daniloski Z, Sanjana N E. Massively parallel Cas13 screens reveal principles for guide RNA design. Nat Biotechnol. 2020 June; 38(6):722-727. doi: 10.1038/s41587-020-0456-9. Epub 2020 Mar. 16.
2. Poling B C, Tsai K, Kang D, Ren L, Kennedy E M, Cullen B R. A lentiviral vector bearing a reverse intron demonstrates superior expression of both proteins and microRNAs. RNA Biol. 2017 Nov. 2; 14(11):1570-1579. doi: 10.1080/15476286.2017.1334755. Epub 2017 Jul. 21.
3. Inglis A J, Guna A, Gálvez-Merchán Á, Pal A, Esantsi T K, Keys H R, Frenkel E M, Oania R, Weissman J S, Voorhees R M. Coupled protein quality control during nonsense-mediated mRNA decay. J Cell Sci. 2023 May 15; 136(10):jcs261216. doi: 10.1242/jcs.261216. Epub 2023 May 23.

### Equivalents and Scope

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure includes embodiments in which more than one, or all of the group members are present in, employed in, or otherwise relevant to a given product or process.

Furthermore, the present disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the present disclosure, or aspects of the present disclosure, is/are referred to as comprising particular elements and/or features, certain embodiments of the present disclosure or aspects of the present disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the present disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the present disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

1. A non-naturally occurring protein, wherein the non-naturally occurring protein comprises an ILF3 sequence, wherein the ILF3 sequence comprises:
(a) a deletion of one or more of the following domains relative to a wild-type ILF3 sequence: GQSY-repeat motif, double-stranded RNA-binding domain 1 (dsRBD1) domain, double-stranded RNA-binding domain 2 (dsRBD2) domain, a nuclear localization signal (NLS), a RGG-repeat motif, an NES domain, a DZF domain, and/or a NVKQ motif (NVKQ), optionally wherein the ILF3 sequence comprises a deletion of one or more of the following domains relative to a wild-type ILF3 sequence: an NES domain, a DZF domain, and/or a NVKQ motif (NVKQ); or
(b) a double-stranded RNA-binding domain 1 (dsRBD1) domain, double-stranded RNA-binding domain 2 (dsRBD2) domain, a nuclear localization signal (NLS), GOSY-repeat domain, and a RGG-repeat motif.

2. (canceled)

3. A fusion protein comprising an ILF3 sequence linked to an RNA-targeting Cas protein, wherein the nuclease activity of the RNA-targeting Cas protein toward target RNA is inactive wherein the ILF3 sequence is the non-naturally occurring protein of claim 1.

4-10. (canceled)

11. The fusion protein of claim 3, wherein the ILF3 sequence comprises an amino acid sequence that is at least 90% identical to one or more of SEQ ID NOs: 1-14, 61, and 69.

12. The fusion protein of claim 3, wherein the RNA-targeting Cas protein comprises an amino acid sequence that is at least 90% identical to SEQ ID NO: 63.

13. The fusion protein of claim 12, wherein the RNA-targeting Cas protein does not comprise SEQ ID NO: 64 and/or does not comprise SEQ ID NO: 65, optionally wherein the Cas protein comprises SEQ ID NO: 80 and/or SEQ ID NO: 81.

14. The fusion protein of claim 3, wherein the fusion protein comprises an amino acid sequence that is at least 90% identical to any one of SEQ ID NOs: 1-14, 61-63, 66-69, and 80-81.

15. An engineered nucleic acid encoding the non-naturally occurring protein of claim 1.

16-18. (canceled)

19. A lipid nanoparticle that encapsulates the non-naturally occurring protein of claim 1.

20. A composition comprising the non-naturally occurring protein of claim 1.

21-39. (canceled)

40. A method of identifying one or more oligonucleotides that are capable of upregulating expression of a gene of interest comprising:
(i) (a) contacting cells with a population of expression vectors, wherein the cells are eukaryotic cells and each expression vector (i) is capable of inducing RNA decay and (ii) encodes an oligonucleotide that is less than 300 nucleotides in length operably linked to a promoter;
(b) identifying a subset of the cells as having increased expression of the gene of interest as compared to control cells; and
(c) detecting one or more oligonucleotides in the subset of the cells, thereby identifying one or more oligonucleotides that are capable of upregulating expression of a gene of interest; or
(ii) (a) immunoprecipitating ILF3 from a eukaryotic cell; and
(b) detecting one or more ribonucleic acids bound to ILF3, there by identifying oligonucleotides capable of upregulating gene expression.

41-61. (canceled)

62. A ribonucleoprotein complex comprising the non-naturally occurring protein of claim 1 and a ribonucleic acid that is less than 300 nucleotides in length.

63. The ribonucleoprotein complex of claim 62, wherein the ribonucleic acid is less than 32 nucleotides in length, optionally wherein the ribonucleic acid comprises a sequence that is at least 90% identical to any one of SEQ ID NOs: 37-40 and 87-88.

64-65. (canceled)

66. A ribonucleoprotein complex comprising the non-naturally occurring protein of claim 1 and a trigger ribonucleic acid.

67-72. (canceled)

73. An engineered nucleic acid that targets an antisense transcript, wherein the ribonucleic acid is the one or more oligonucleotides that are capable of upregulating expression of a gene of interest identified by the method of claim 40.

74-75. (canceled)

76. A host cell comprising the non-naturally occurring protein of claim 1.

77-79. (canceled)

80. A kit comprising the non-naturally occurring protein of claim 1.

81. A method of increasing expression of a gene of interest comprising administering to a cell, tissue, or organ the non-naturally occurring protein of claim 1.

82-83. (canceled)

84. A method of treating a disease characterized by a decrease in expression of a gene of interest comprising:
(a) administering to the subject the non-naturally occurring protein of claim 1; and/or
(b) administering to a subject a trigger nucleic acid to increase expression of the gene of interest, optionally wherein the trigger nucleic acid is an antisense oligonucleotide or a trigger ribonucleic acid and/or the trigger nucleic acid comprises a sequence that is at least 90% identical to any one of SEQ ID NO: 37-40, 49-55, 58-60, and 87-88.

85. (canceled)

86. A method comprising:
a) increasing expression of a gene in a cell comprising administering a trigger nucleic acid to increase expression of the gene of interest, optionally wherein the trigger nucleic acid is an antisense oligonucleotide or a trigger ribonucleic acid and/or the trigger nucleic acid comprises a sequence that is at least 90% identical to any one of SEQ ID NOs: 37-40, 49-55, 58-60, and 87-88;
b) treating a disease characterized by a decrease in expression of a gene of interest comprising deactivating one or more antisense transcripts of the gene of interest to increase expression of the gene of interest in a subject;
c) inducing RNA decay of the mRNA of a first gene in a cell to increase expression of a second gene in a cell, wherein the first gene is a perturbed gene set forth in Table 7 and the second gene is a corresponding adapting gene set forth in Table 7; and/or
d) inducing RNA decay of the mRNA of ACTG1 to increase expression of a second gene in a cell, wherein the second gene is a corresponding adapting gene set forth in Table 8.

87-96. (canceled)

97. Use of the non-naturally occurring protein of claim 1 to treat a subject with a disease.

98-102. (canceled)


SEQUENCE LISTING (117 sequences)

SEQ ID NO: 1 - protein, 898 aa, Homo sapiens
MRPMRIFVNDDRHVMAKHSSVYPTQEELEAVQNMVSHTERALKAVSDWIDEQEKGSSEQAESDNMDVPPEDDSKEGAGEQKTEHMTRTLRGVMRVGLVAKGLLLKGDLDLELVLLCKEKPTTALLDKVADNLAIQLAAVTEDKYEILQSVDDAAIVIKNTKEPPLSLTIHLTSPVVREEMEKVLAGETLSVNDPPDVLDRQKCLAALASLRHAKWFQARANGLKSCVIVIRVLRDLCTRVPTWGPLRGWPLELLCEKSIGTANRPMGAGEALRRVLECLASGIVMPDGSGIYDPCEKEATDAIGHLDRQQREDITQSAQHALRLAAFGQLHKVLGMDPLPSKMPKKPKNENPVDYTVQIPPSTTYAITPMKRPMEEDGEEKSPSKKKKKIQKKEEKAEPPQAMNALMRLNQLKPGLQYKLVSQTGPVHAPIFTMSVEVDGNSFEASGPSKKTAKLHVAVKVLQDMGLPTGAEGRDSSKGEDSAEETEAKPAVVAPAPVVEAVSTPSAAFPSDATAENVKQQGPILTKHGKNPVMELNEKRRGLKYELISETGGSHDKRFVMEVEVDGQKFQGAGSNKKVAKAYAALAALEKLFPDTPLALDANKKKRAPVPVRGGPKFAAKPHNPGFGMGGPMHNEVPPPPNLRGRGRGGSIRGRGRGRGFGGANHGGYMNAGAGYGSYGYGGNSATAGYSQFYSNGGHSGNASGGGGGGGGGSSGYGSYYQGDNYNSPVPPKHAGKKQPHGGQQKPSYGSGYQSHQGQQQSYNQSPYSNYGPPQGKQKGYNHGQGSYSYSNSYNSPGGGGGSDYNYESKFNYSGSGGRSGGNSYGSGGASYNPGSHGGYGGGSGGGSSYQGKQGGYSQSNYNSPGSGQNYSGPPSSYQSSQGGYGRNADHSMNYQYR

SEQ ID NO: 2 - protein, 706 aa, Homo sapiens
MRPMRIFVNDDRHVMAKHSSVYPTQEELEAVQNMVSHTERALKAVSDWIDEQEKGSSEQAESDNMDVPPEDDSKEGAGEQKTEHMTRTLRGVMRVGLVAKGLLLKGDLDLELVLLCKEKPTTALLDKVADNLAIQLAAVTEDKYEILQSVDDAAIVIKNTKEPPLSLTIHLTSPVVREEMEKVLAGETLSVNDPPDVLDRQKCLAALASLRHAKWFQARANGLKSCVIVIRVLRDLCTRVPTWGPLRGWPLELLCEKSIGTANRPMGAGEALRRVLECLASGIVMPDGSGIYDPCEKEATDAIGHLDRQQREDITQSAQHALRLAAFGQLHKVLGMDPLPSKMPKKPKNENPVDYTVQIPPSTTYAITPMKRPMEEDGEEKSPSKKKKKIQKKEEKAEPPQAMNALMRLNQLKPGLQYKLVSQTGPVHAPIFTMSVEVDGNSFEASGPSKKTAKLHVAVKVLQDMGLPTGAEGRDSSKGEDSAEETEAKPAVVAPAPVVEAVSTPSAAFPSDATAENVKQQGPILTKHGKNPVMELNEKRRGLKYELISETGGSHDKRFVMEVEVDGQKFQGAGSNKKVAKAYAALAALEKLFPDTPLALDANKKKRAPVPVRGGPKFAAKPHNPGFGMGGPMHNEVPPPPNLRGRGRGGSIRGRGRGRGFGGANHGGYMNAGAGYGSYGYGGNSATAGYSDFFTDCYGYHDFGSS

SEQ ID NO: 3 - protein, 898 aa, Mus musculus
MRPMRIFVNDDRHVMAKHSSVYPTQEELEAVQNMVSHTERALKAVSDWIDEQEKGNSELSEAENMDTPPDDESKEGAGEQKAEHMTRTLRGVMRVGLVAKGLLLKGDLDLELVLLCKEKPTTALLDKVADNLAIQLTTVTEDKYEILQSVDDAAIVIKNTKEPPLSLTIHLTSPVVREEMEKVLAGETLSVNDPPDVLDRQKCLAALASLRHAKWFQARANGLKSCVIVIRVLRDLCTRVPTWGPLRGWPLELLCEKSIGTANRPMGAGEALRRVLECLASGIVMPDGSGIYDPCEKEATDAIGHLDRQQREDITQSAQHALRLAAFGQLHKVLGMDPLPSKMPKKPKNENPVDYTVQIPPSTTYAITPMKRPMEEDGEEKSPSKKKKKIQKKEEKADPPQAMNALMRLNQLKPGLQYKLISQTGPVHAPIFTMSVEVDGSNFEASGPSKKTAKLHVAVKVLQDMGLPTGAEGRDSSKGEDSAEESDGKPAIVAPPPVVEAVSNPSSVFPSDATTEQGPILTKHGKNPVMELNEKRRGLKYELISETGGSHDKRFVMEVEVDGQKFQGAGSNKKVAKAYAALAALEKLFPDTPLALEANKKKRTPVPVRGGPKFAAKPHNPGFGMGGPMHNEVPPPPNIRGRGRGGNIRGRGRGRGFGGANHGGGYMNAGAGYGSYGYSSNSATAGYSQFYSNGGHSGNAGGGGSGGGGGSSSYSSYYQGDSYNSPVPPKHAGKKPLHGGQQKASYSSGYQSHQGQQQPYNQSQYSSYGTPQGKQKGYGHGQGSYSSYSNSYNSPGGGGGSDYSYDSKFNYSGSGGRSGGNSYGSSGSSSYNTGSHGGYGTGSGGSSSYQGKQGGYSSQSNYSSPGSSQSYSGPASSYQSSQGGYSRNTEHSMNYQYR

SEQ ID NO: 4 - protein, 703 aa, Mus musculus
MRPMRIFVNDDRHVMAKHSSVYPTQEELEAVQNMVSHTERALKAVSDWIDEQEKGNSELSEAENMDTPPDDESKEGAGEQKAEHMTRTLRGVMRVGLVAKGLLLKGDLDLELVLLCKEKPTTALLDKVADNLAIQLTTVTEDKYEILQSVDDAAIVIKNTKEPPLSLTIHLTSPVVREEMEKVLAGETLSVNDPPDVLDRQKCLAALASLRHAKWFQARANGLKSCVIVIRVLRDLCTRVPTWGPLRGWPLELLCEKSIGTANRPMGAGEALRRVLECLASGIVMPDGSGIYDPCEKEATDAIGHLDRQQREDITQSAQHALRLAAFGQLHKVLGMDPLPSKMPKKPKNENPVDYTVQIPPSTTYAITPMKRPMEEDGEEKSPSKKKKKIQKKEEKADPPQAMNALMRLNQLKPGLQYKLISQTGPVHAPIFTMSVEVDGSNFEASGPSKKTAKLHVAVKVLQDMGLPTGAEGRDSSKGEDSAEESDGKPAIVAPPPVVEAVSNPSSVFPSDATTEQGPILTKHGKNPVMELNEKRRGLKYELISETGGSHDKRFVMEVEVDGQKFQGAGSNKKVAKAYAALAALEKLFPDTPLALEANKKKRTPVPVRGGPKFAAKPHNPGFGMGGPMHNEVPPPPNIRGRGRGGNIRGRGRGRGFGGANHGGGYMNAGAGYGSYGYSSNSATAGYSDFFTDCYGYHDFGAS

SEQ ID NO: 5 - protein, 529 aa, synthetic construct
MKRPMEEDGEEKSPSKKKKKIQKKEEKADPPQAMNALMRLNQLKPGLQYKLISQTGPVHAPIFTMSVEVDGSNFEASGPSKKTAKLHVAVKVLQDMGLPTGAEGRDSSKGEDSAEESDGKPAIVAPPPVVEAVSNPSSVFPSDATTEQGPILTKHGKNPVMELNEKRRGLKYELISETGGSHDKRFVMEVEVDGQKFQGAGSNKKVAKAYAALAALEKLFPDTPLALEANKKKRTPVPVRGGPKFAAKPHNPGFGMGGPMHNEVPPPPNIRGRGRGGNIRGRGRGRGFGGANHGGGYMNAGAGYGSYGYSSNSATAGYSQFYSNGGHSGNAGGGGSGGGGGSSSYSSYYQGDSYNSPVPPKHAGKKPLHGGQQKASYSSGYQSHQGQQQPYNQSQYSSYGTPQGKQKGYGHGQGSYSSYSNSYNSPGGGGGSDYSYDSKFNYSGSGGRSGGNSYGSSGSSSYNTGSHGGYGTGSGGSSSYQGKQGGYSSQSNYSSPGSSQSYSGPASSYQSSQGGYSRNTEHSMNYQYR

SEQ ID NO: 6 - protein, 24 aa, synthetic construct
KRPMEEDGEEKSPSKKKKKIQKKE

SEQ ID NO: 7 - protein, 4 aa, synthetic construct
NVKQ

SEQ ID NO: 8 - protein, 62 aa, synthetic construct
NALMRLNQLKPGLQYKLISQTGPVHAPIFTMSVEVDGSNFEASGPSKKTAKLHVAVKVLQDM

SEQ ID NO: 9 - protein, 369 aa, synthetic construct
RPMRIFVNDDRHVMAKHSSVYPTQEELEAVQNMVSHTERALKAVSDWIDEQEKGNSELSEAENMDTPPDDESKEGAGEQKAEHMTRTLRGVMRVGLVAKGLLLKGDLDLELVLLCKEKPTTALLDKVADNLAIQLTTVTEDKYEILQSVDDAAIVIKNTKEPPLSLTIHLTSPVVREEMEKVLAGETLSVNDPPDVLDRQKCLAALASLRHAKWFQARANGLKSCVIVIRVLRDLCTRVPTWGPLRGWPLELLCEKSIGTANRPMGAGEALRRVLECLASGIVMPDGSGIYDPCEKEATDAIGHLDRQQREDITQSAQHALRLAAFGQLHKVLGMDPLPSKMPKKPKNENPVDYTVQIPPSTTYAITPM

SEQ ID NO: 10 - protein, 62 aa, synthetic construct
NPVMELNEKRRGLKYELISETGGSHDKRFVMEVEVDGQKFQGAGSNKKVAKAYAALAALEKL

SEQ ID NO: 11 - protein, 17 aa, synthetic construct
RGRGRGGNIRGRGRGRG

SEQ ID NO: 12 - protein, 62 aa, synthetic construct
NALMRLNQLKPGLQYKLVSQTGPVHAPIFTMSVEVDGNSFEASGPSKKTAKLHVAVKVLQDM

SEQ ID NO: 13 - protein, 369 aa, synthetic construct
RPMRIFVNDDRHVMAKHSSVYPTQEELEAVQNMVSHTERALKAVSDWIDEQEKGSSEQAESDNMDVPPEDDSKEGAGEQKTEHMTRTLRGVMRVGLVAKGLLLKGDLDLELVLLCKEKPTTALLDKVADNLAIQLAAVTEDKYEILQSVDDAAIVIKNTKEPPLSLTIHLTSPVVREEMEKVLAGETLSVNDPPDVLDRQKCLAALASLRHAKWFQARANGLKSCVIVIRVLRDLCTRVPTWGPLRGWPLELLCEKSIGTANRPMGAGEALRRVLECLASGIVMPDGSGIYDPCEKEATDAIGHLDRQQREDITQSAQHALRLAAFGQLHKVLGMDPLPSKMPKKPKNENPVDYTVQIPPSTTYAITPM

SEQ ID NO: 14 - protein, 17 aa, synthetic construct
RGRGRGGSIRGRGRGRG

SEQ ID NO: 15 - DNA, 19 bp, synthetic construct
  source 1..19: mol_type = other DNA
gcagagagaagatgaccca

SEQ ID NO: 16 - DNA, 19 bp, synthetic construct
  source 1..19: mol_type = other DNA
cgcttcaatgtccctgcca

SEQ ID NO: 17 - DNA, 23 bp, synthetic construct
  source 1..23: mol_type = other DNA
accatgtgtgaagaagagaccac

SEQ ID NO: 18 - DNA, 23 bp, synthetic construct
  source 1..23: mol_type = other DNA
aggctattcctttgtgaccacag

SEQ ID NO: 19 - DNA, 19 bp, synthetic construct
  source 1..19: mol_type = other DNA
aacaaagaaagccagccag

SEQ ID NO: 20 - DNA, 23 bp, synthetic construct
  source 1..23: mol_type = other DNA
gaaaaatcattcagagcagaccc

SEQ ID NO: 21 - DNA, 19 bp, synthetic construct
  source 1..19: mol_type = other DNA
cagagcaatataccccaag

SEQ ID NO: 22 - DNA, 23 bp, synthetic construct
  source 1..23: mol_type = other DNA
ctgatgtctagggcggcccacaa

SEQ ID NO: 23 - DNA, 23 bp, synthetic construct
  source 1..23: mol_type = other DNA
tacttgagagttaggatcccacg

SEQ ID NO: 24 - DNA, 23 bp, synthetic construct
  source 1..23: mol_type = other DNA
agcaacatacatggcagggacat

SEQ ID NO: 25 - DNA, 23 bp, synthetic construct
  source 1..23: mol_type = other DNA
agtatagttccaatacagctcac

SEQ ID NO: 26 - DNA, 23 bp, synthetic construct
  source 1..23: mol_type = other DNA
actgtctgaaaaatgccacagca

SEQ ID NO: 27 - DNA, 23 bp, synthetic construct
  source 1..23: mol_type = other DNA
ggtaacacagagcagcaccagct

SEQ ID NO: 28 - DNA, 19 bp, synthetic construct
  source 1..19: mol_type = other DNA
ataagttcaaggccaccat

SEQ ID NO: 29 - DNA, 19 bp, synthetic construct
  source 1..19: mol_type = other DNA
catgagaaaaaagcgccaa

SEQ ID NO: 30 - DNA, 23 bp, synthetic construct
  source 1..23: mol_type = other DNA
gtagaaataattgaacagccaag

SEQ ID NO: 31 - DNA, 23 bp, synthetic construct
  source 1..23: mol_type = other DNA
acaagctgatgtacaccgccaag

SEQ ID NO: 32 - DNA, 23 bp, synthetic construct
  source 1..23: mol_type = other DNA
tccagcaagaacaagccacacgt

SEQ ID NO: 33 - DNA, 23 bp, synthetic construct
  source 1..23: mol_type = other DNA
agcacaagaaagaccaccccgat

SEQ ID NO: 34 - DNA, 23 bp, synthetic construct
  source 1..23: mol_type = other DNA
aacaattagaggaagcaagccca

SEQ ID NO: 35 - DNA, 22 bp, synthetic construct
  source 1..22: mol_type = other DNA
tcaccagaagcgtaccatactc

SEQ ID NO: 36 - RNA, 21 nt, synthetic construct
  source 1..21: mol_type = other RNA
ataaaggagaagctgtgctat

SEQ ID NO: 37 - RNA, 23 nt, synthetic construct
  source 1..23: mol_type = other RNA
tgagcaagaaatggctactgctg

SEQ ID NO: 38 - RNA, 24 nt, synthetic construct
  source 1..24: mol_type = other RNA
cgtgacataaaggagaagctgtgc

SEQ ID NO: 39 - RNA, 27 nt, synthetic construct
  source 1..27: mol_type = other RNA
tgtgctatgttgccctggattttgagc

SEQ ID NO: 40 - RNA, 31 nt, synthetic construct
  source 1..31: mol_type = other RNA
atgttgccctggattttgagcaagaaatggc

SEQ ID NO: 41 - RNA, 32 nt, synthetic construct
  source 1..32: mol_type = other RNA
ctggattttgagcaagaaatggctactgctgc

SEQ ID NO: 42 - RNA, 40 nt, synthetic construct
  source 1..40: mol_type = other RNA
ataaaggagaagctgtgctatgttgccctggattttgagc

SEQ ID NO: 43 - RNA, 53 nt, synthetic construct
  source 1..53: mol_type = other RNA
ataaaggagaagctgtgctatgttgccctggattttgagcaagaaatggctac

SEQ ID NO: 44 - RNA, 60 nt, synthetic construct
  source 1..60: mol_type = other RNA
tgacataaaggagaagctgtgctatgttgccctggattttgagcaagaaatggctactgc

SEQ ID NO: 45 - RNA, 24 nt, synthetic construct
  source 1..24: mol_type = other RNA
cgtgccctgccggcgccgctgtgc

SEQ ID NO: 46 - RNA, 27 nt, synthetic construct
  source 1..27: mol_type = other RNA
cgcgccacgccgccccggacccggagc

SEQ ID NO: 47 - RNA, 31 nt, synthetic construct
  source 1..31: mol_type = other RNA
acgccgccccggacccggagcaagaaacggc

SEQ ID NO: 48 - DNA, 16 bp, synthetic construct
  source 1..16: mol_type = other DNA
  modified_base 1..16: mod_base = OTHER; note = Phosphorothioate bond
ggctactacgccgtca

SEQ ID NO: 49 - DNA, 16 bp, synthetic construct
  source 1..16: mol_type = other DNA
  modified_base 1..16: mod_base = OTHER; note = Phosphorothioate bond
tacgagacatcaagga

SEQ ID NO: 50 - DNA, 16 bp, synthetic construct
  source 1..16: mol_type = other DNA
  modified_base 1..16: mod_base = OTHER; note = Phosphorothioate bond
ccacagcagcttcatc

SEQ ID NO: 51 - DNA, 16 bp, synthetic construct
  source 1..16: mol_type = other DNA
  modified_base 1..16: mod_base = OTHER; note = Phosphorothioate bond
acgagacatcaaggag

SEQ ID NO: 52 - DNA, 16 bp, synthetic construct
  source 1..16: mol_type = other DNA
  modified_base 1..16: mod_base = OTHER; note = Phosphorothioate bond
tcaaggagaagctgtg

SEQ ID NO: 53 - DNA, 16 bp, synthetic construct
  source 1..16: mol_type = other DNA
  modified_base 1..16: mod_base = OTHER; note = Phosphorothioate bond
ggagaagctgtgctat

SEQ ID NO: 54 - DNA, 16 bp, synthetic construct
  source 1..16: mol_type = other DNA
  modified_base 1..16: mod_base = OTHER; note = Phosphorothioate bond
ggatttcgagaatgag

SEQ ID NO: 55 - DNA, 16 bp, synthetic construct
  source 1..16: mol_type = other DNA
  modified_base 1..16: mod_base = OTHER; note = Phosphorothioate bond
gctgtgctatgtagcc

SEQ ID NO: 56 - DNA, 20 bp, synthetic construct
  source 1..20: mol_type = other DNA
  modified_base 1..20: mod_base = OTHER; note = Phosphorothioate bond
  misc_feature 1..5: note = RNA
  misc_feature 6..15: note = DNA
  misc_feature 16..20: note = RNA
  modified_base 1..2: mod_base = OTHER; note = 2'-O-methoxyethylcytidine
  modified_base 3: mod_base = OTHER; note = 2'-O-methoxyethyluridine
  modified_base 4: mod_base = OTHER; note = 2'-O-methoxyethyladenosine
  modified_base 5: mod_base = OTHER; note = 2'-O-methoxyethyluridine
  modified_base 16: mod_base = OTHER; note = 2'-O-methoxyethyladenosine
  modified_base 17..18: mod_base = OTHER; note = 2'-O-methoxyethylguanosine
  modified_base 19..20: mod_base = OTHER; note = 2'-O-methoxyethyladenosine
cctataggactatccaggaa

SEQ ID NO: 57 - DNA, 20 bp, synthetic construct
  source 1..20: mol_type = other DNA
  modified_base 1..20: mod_base = OTHER; note = Phosphorothioate bond
  misc_feature 1..5: note = RNA
  misc_feature 6..15: note = DNA
  misc_feature 16..20: note = RNA
  modified_base 1: mod_base = OTHER; note = 2'-O-methoxyethyladenosine
  modified_base 2: mod_base = OTHER; note = 2'-O-methoxyethylguanosine
  modified_base 3..5: mod_base = OTHER; note = 2'-O-methoxyethylcytidine
  modified_base 16: mod_base = OTHER; note = 2'-O-methoxyethylguanosine
  modified_base 17..18: mod_base = OTHER; note = 2'-O-methoxyethyladenosine
  modified_base 19: mod_base = OTHER; note = 2'-O-methoxyethyluridine
  modified_base 20: mod_base = OTHER; note = 2'-O-methoxyethylguanosine
agccctggatttcgagaatg

SEQ ID NO: 58 - DNA, 20 bp, synthetic construct
  source 1..20: mol_type = other DNA
  modified_base 1..20: mod_base = OTHER; note = Phosphorothioate bond
  misc_feature 1..5: note = RNA
  misc_feature 6..15: note = DNA
  misc_feature 16..20: note = DNA
  modified_base 1: mod_base = OTHER; note = 2'-O-methoxyethyluridine
  modified_base 2: mod_base = OTHER; note = 2'-O-methoxyethylguanosine
  modified_base 3: mod_base = OTHER; note = 2'-O-methoxyethyluridine
  modified_base 4: mod_base = OTHER; note = 2'-O-methoxyethyladenosine
  modified_base 5: mod_base = OTHER; note = 2'-O-methoxyethylcytidine
  modified_base 16..17: mod_base = OTHER; note = 2'-O-methoxyethylguanosine
  modified_base 18: mod_base = OTHER; note = 2'-O-methoxyethyladenosine
  modified_base 19: mod_base = OTHER; note = 2'-O-methoxyethylguanosine
  modified_base 20: mod_base = OTHER; note = 2'-O-methoxyethyladenosine
tgtacgagacatcaaggaga

SEQ ID NO: 59 - DNA, 20 bp, synthetic construct
  source 1..20: mol_type = other DNA
  modified_base 1..20: mod_base = OTHER; note = Phosphorothioate bond
  misc_feature 1..5: note = RNA
  misc_feature 6..15: note = DNA
  misc_feature 16..20: note = RNA
  modified_base 1: mod_base = OTHER; note = 2'-O-methoxyethyladenosine
  modified_base 2: mod_base = OTHER; note = 2'-O-methoxyethylcytidine
  modified_base 3: mod_base = OTHER; note = 2'-O-methoxyethylguanosine
  modified_base 4: mod_base = OTHER; note = 2'-O-methoxyethyladenosine
  modified_base 5: mod_base = OTHER; note = 2'-O-methoxyethylguanosine
  modified_base 16: mod_base = OTHER; note = 2'-O-methoxyethylguanosine
  modified_base 17..18: mod_base = OTHER; note = 2'-O-methoxyethyladenosine
  modified_base 19: mod_base = OTHER; note = 2'-O-methoxyethylguanosine
  modified_base 20: mod_base = OTHER; note = 2'-O-methoxyethylcytidine
acgagacatcaaggagaagc

SEQ ID NO: 60 - DNA, 20 bp, synthetic construct
  source 1..20: mol_type = other DNA
  modified_base 1..20: mod_base = OTHER; note = Phosphorothioate bond
  misc_feature 1..5: note = RNA
  misc_feature 6..15: note = DNA
  misc_feature 16..20: note = RNA
  modified_base 1..2: mod_base = OTHER; note = 2'-O-methoxyethyladenosine
  modified_base 3..4: mod_base = OTHER; note = 2'-O-methoxyethylguanosine
  modified_base 5: mod_base = OTHER; note = 2'-O-methoxyethyladenosine
  modified_base 16: mod_base = OTHER; note = 2'-O-methoxyethyluridine
  modified_base 17: mod_base = OTHER; note = 2'-O-methoxyethyladenosine
  modified_base 18: mod_base = OTHER; note = 2'-O-methoxyethyluridine
  modified_base 19: mod_base = OTHER; note = 2'-O-methoxyethylguanosine
  modified_base 20: mod_base = OTHER; note = 2'-O-methoxyethyluridine
aaggagaagctgtgctatgt

SEQ ID NO: 61 - protein, 211 aa, synthetic construct
SQFYSNGGHSGNAGGGGSGGGGGSSSYSSYYQGDSYNSPVPPKHAGKKPLHGGQQKASYSSGYQSHQGQQQPYNQSQYSSYGTPQGKQKGYGHGQGSYSSYSNSYNSPGGGGGSDYSYDSKFNYSGSGGRSGGNSYGSSGSSSYNTGSHGGYGTGSGGSSSYQGKQGGYSSQSNYSSPGSSQSYSGPASSYQSSQGGYSRNTEHSMNYQYR

SEQ ID NO: 62 - protein, 2241 aa, synthetic construct
MSPKKKRKVEASIEKKKSFAKGMGVKSTLVSGSKVYMTTFAEGSDARLEKIVEGDSIRSVNEGEAFSAEMADKNAGYKIGNAKFSHPKGYAVVANNPLYTGPVQQDMLGLKETLEKRYFGESADGNDNICIQVIHNILDIEKILAEYITNAAYAVNNISGLDKDIIGFGKFSTVYTYDEFKDPEHHRAAFNNNDKLINAIKAQYDEFDNFLDNPRLGYFGQAFFSKEGRNYIINYGNECYDILALLSGLAHWVVANNEEESRISRTWLYNLDKNLDNEYISTLNYLYDRITNELTNSFSKNSAANVNYIAETLGINPAEFAEQYFRFSIMKEQKNLGFNITKLREVMLDRKDMSEIRKNHKVFDSIRTKVYTMMDFVIYRYYIEEDAKVAAANKSLPDNEKSLSEKDIFVINLRGSFNDDQKDALYYDEANRIWRKLENIMHNIKEFRGNKTREYKKKDAPRLPRILPAGRDVSAFSKLMYALTMFLDGKEINDLLTTLINKFDNIQSFLKVMPLIGVNAKFVEEYAFFKDSAKIADELRLIKSFARMGEPIADARRAMYIDAIRILGTNLSYDELKALADTFSLDENGNKLKKGKHGMRNFIINNVISNKRFHYLIRYGDPAHLHEIAKNEAVVKFVLGRIADIQKKQGQNGKNQIDRYYETCIGKDKGKSVSEKVDALTKIITGMNYDQFDKKRSVIEDTGRENAEREKFKKIISLYLTVIYHILKNIVNINARYVIGFHCVERDAQLYKEKGYDINLKKLEEKGFSSVTKLCAGIDETAPDKRKDVEKEMAERAKESIDSLESANPKLYANYIKYSDEKKAEEFTRQINREKAKTALNAYLRNTKWNVIIREDLLRIDNKTCTLFANKAVALEVARYVHAYINDIAEVNSYFQLYHYIMQRIIMNERYEKSSGKVSEYFDAVNDEKKYNDRLLKLLCVPFGYCIPRFKNLSIEALFDRNEAAKFDKEKKKVSGNSGSGPKKKRKVAAAYPYDVPDYAASGGPSSGAPPPSGGSPAGSPTSTEEGTSESATPESGPGTSTEPSEGSAPGSPAGSPTSTEEGTSTEPSEGSAPGTSTEPSEMRPMRIFVNDDRHVMAKHSSVYPTQEELEAVQNMVSHTERALKAVSDWIDEQEKGNSELSEAENMDTPPDDESKEGAGEQKAEHMTRTLRGVMRVGLVAKGLLLKGDLDLELVLLCKEKPTTALLDKVADNLAIQLTTVTEDKYEILQSVDDAAIVIKNTKEPPLSLTIHLTSPVVREEMEKVLAGETLSVNDPPDVLDRQKCLAALASLRHAKWFQARANGLKSCVIVIRVLRDLCTRVPTWGPLRGWPLELLCEKSIGTANRPMGAGEALRRVLECLASGIVMPDGSGIYDPCEKEATDAIGHLDRQQREDITQSAQHALRLAAFGQLHKVLGMDPLPSKMPKKPKNENPVDYTVQIPPSTTYAITPMKRPMEEDGEEKSPSKKKKKIQKKEEKADPPQAMNALMRLNQLKPGLQYKLISQTGPVHAPIFTMSVEVDGSNFEASGPSKKTAKLHVAVKVLQDMGLPTGAEGRDSSKGEDSAEESDGKPAIVAPPPVVEAVSNPSSVFPSDATTEQGPILTKHGKNPVMELNEKRRGLKYELISETGGSHDKRFVMEVEVDGQKFQGAGSNKKVAKAYAALAALEKLFPDTPLALEANKKKRTPVPVRGGPKFAAKPHNPGFGMGGPMHNEVPPPPNIRGRGRGGNIRGRGRGRGFGGANHGGGYMNAGAGYGSYGYSSNSATAGYSQFYSNGGHSGNAGGGGSGGGGGSSSYSSYYQGDSYNSPVPPKHAGKKPLHGGQQKASYSSGYQSHQGQQQPYNQSQYSSYGTPQGKQKGYGHGQGSYSSYSNSYNSPGGGGGSDYSYDSKFNYSGSGGRSGGNSYGSSGSSSYNTGSHGGYGTGSGGSSSYQGKQGGYSSQSNYSSPGSSQSYSGPASSYQSSQGGYSRNTEHSMNYQYRASGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 63 - protein, 966 aa, synthetic construct
IEKKKSFAKGMGVKSTLVSGSKVYMTTFAEGSDARLEKIVEGDSIRSVNEGEAFSAEMADKNAGYKIGNAKFSHPKGYAVVANNPLYTGPVQQDMLGLKETLEKRYFGESADGNDNICIQVIHNILDIEKILAEYITNAAYAVNNISGLDKDIIGFGKFSTVYTYDEFKDPEHHRAAFNNNDKLINAIKAQYDEFDNFLDNPRLGYFGQAFFSKEGRNYIINYGNECYDILALLSGLAHWVVANNEEESRISRTWLYNLDKNLDNEYISTLNYLYDRITNELTNSFSKNSAANVNYIAETLGINPAEFAEQYFRFSIMKEQKNLGFNITKLREVMLDRKDMSEIRKNHKVFDSIRTKVYTMMDFVIYRYYIEEDAKVAAANKSLPDNEKSLSEKDIFVINLRGSFNDDQKDALYYDEANRIWRKLENIMHNIKEFRGNKTREYKKKDAPRLPRILPAGRDVSAFSKLMYALTMFLDGKEINDLLTTLINKFDNIQSFLKVMPLIGVNAKFVEEYAFFKDSAKIADELRLIKSFARMGEPIADARRAMYIDAIRILGTNLSYDELKALADTFSLDENGNKLKKGKHGMRNFIINNVISNKRFHYLIRYGDPAHLHEIAKNEAVVKFVLGRIADIQKKQGQNGKNQIDRYYETCIGKDKGKSVSEKVDALTKIITGMNYDQFDKKRSVIEDTGRENAEREKFKKIISLYLTVIYHILKNIVNINARYVIGFHCVERDAQLYKEKGYDINLKKLEEKGFSSVTKLCAGIDETAPDKRKDVEKEMAERAKESIDSLESANPKLYANYIKYSDEKKAEEFTRQINREKAKTALNAYLRNTKWNVIIREDLLRIDNKTCTLFANKAVALEVARYVHAYINDIAEVNSYFQLYHYIMQRIIMNERYEKSSGKVSEYFDAVNDEKKYNDRLLKLLCVPFGYCIPRFKNLSIEALFDRNEAAKFDKEKKKVSGNS

SEQ ID NO: 64 - protein, 6 aa, synthetic construct
RHWVVH

SEQ ID NO: 65 - protein, 6 aa, synthetic construct
RNKAVH

SEQ ID NO: 66 - protein, 80 aa, synthetic construct
GGPSSGAPPPSGGSPAGSPTSTEEGTSESATPESGPGTSTEPSEGSAPGSPAGSPTSTEEGTSTEPSEGSAPGTSTEPSE

SEQ ID NO: 67 - protein, 7 aa, synthetic construct
PKKKRKV

SEQ ID NO: 68 - protein, 9 aa, synthetic construct
YPYDVPDYA

SEQ ID NO: 69 - protein, 208 aa, synthetic construct
SQFYSNGGHSGNASGGGGGGGGGSSGYGSYYQGDNYNSPVPPKHAGKKQPHGGQQKPSYGSGYQSHQGQQQSYNQSPYSNYGPPQGKQKGYNHGQGSYSYSNSYNSPGGGGGSDYNYESKFNYSGSGGRSGGNSYGSGGASYNPGSHGGYGGGSGGGSSYQGKQGGYSQSNYNSPGSGQNYSGPPSSYQSSQGGYGRNADHSMNYQYR

SEQ ID NO: 70 - DNA, 20 bp, synthetic construct
  source 1..20: mol_type = other DNA
gatcagtcaacgggggacat

SEQ ID NO: 71 - DNA, 21 bp, synthetic construct
  source 1..21: mol_type = other DNA
cattttggggctgtactgctt

SEQ ID NO: 72 - DNA, 19 bp, synthetic construct
  source 1..19: mol_type = other DNA
ccgccctagacatcagggt

SEQ ID NO: 73 - DNA, 21 bp, synthetic construct
  source 1..21: mol_type = other DNA
tcttctggtgctactcgaagc

SEQ ID NO: 74 - DNA, 19 bp, synthetic construct
  source 1..19: mol_type = other DNA
taaagccaagcaccgtcag

SEQ ID NO: 75 - DNA, 21 bp, synthetic construct
  source 1..21: mol_type = other DNA
gatttccctcaaggctgtgat

SEQ ID NO: 76 - DNA, 18 bp, synthetic construct
  source 1..18: mol_type = other DNA
agacccttcgtggaggag

SEQ ID NO: 77 - DNA, 18 bp, synthetic construct
  source 1..18: mol_type = other DNA
tcggttttgggagtggtg

SEQ ID NO: 78 - DNA, 20 bp, synthetic construct
  source 1..20: mol_type = other DNA
atacctgccagatgaaaaag

SEQ ID NO: 79 - DNA, 20 bp, synthetic construct
  source 1..20: mol_type = other DNA
tcagtaaagtgaccacaatc

SEQ ID NO: 80 - protein, 6 aa, synthetic construct
AHWVVA

SEQ ID NO: 81 - protein, 6 aa, synthetic construct
ANKAVA

SEQ ID NO: 82 - DNA, 75 bp, synthetic construct
  source 1..75: mol_type = other DNA
tgttcgtgacataaaggagaagctgtgctatgttgccctggattttgagcaagaaatggctactgctgcatcatc

SEQ ID NO: 83 - DNA, 100 bp, synthetic construct
  source 1..100: mol_type = other DNA
accacagctgagagagaaattgtacgagacatcaaggagaagctgtgctatgtagccctggatttcgagaatgagatggccacagcagcttcatcttctt

SEQ ID NO: 84 - DNA, 100 bp, synthetic construct
  source 1..100: mol_type = other DNA
aagaagatgaagctgctgtggccatctcattctcgaaatccagggctacatagcacagcttctccttgatgtctcgtacaatttctctctcagctgtggt

SEQ ID NO: 85 - protein, 25 aa, synthetic construct
SSAATAMEQEFDLAVYCLKEKIDEV

SEQ ID NO: 86 - DNA, 75 bp, synthetic construct
  source 1..75: mol_type = other DNA
gatgatgcagcagtagccatttcttgctcaaaatccagggcaacatagcacagcttctcctttatgtcacgaaca

SEQ ID NO: 87 - RNA, 75 nt, synthetic construct
  source 1..75: mol_type = other RNA
tgttcgtgacataaaggagaagctgtgctatgttgccctggattttgagcaagaaatggctactgctgcatcatc

SEQ ID NO: 88 - RNA, 81 nt, synthetic construct
  source 1..81: mol_type = other RNA
atgtgttcgtgacataaaggagaagctgtgctatgttgccctggattttgagcaagaaatggctactgctgcatcatctaa

SEQ ID NO: 89-108 - skipped (20 entries)

SEQ ID NO: 109 - DNA, 109 bp, synthetic construct
  source 1..109: mol_type = other DNA
gtgaattgtaatacgactcactatagggatgtgttcgtgacataaaggagaagctgtgctatgttgccctggattttgagcaagaaatggctactgctgcatcatctaa

SEQ ID NO: 110 - DNA, 109 bp, synthetic construct
  source 1..109: mol_type = other DNA
gtgaattgtaatacgactcactatagggatgcaatttcagccctcttatcctcggcgttgtgtgtcaagtgacgtagacctagattgactctatgacggtatctgctaa

SEQ ID NO: 111 - DNA, 11 bp, synthetic construct
  source 1..11: mol_type = other DNA
ccaccttgttg

SEQ ID NO: 112 - DNA, 44 bp, synthetic construct
  source 1..44: mol_type = other DNA
gtttcagagcgagacgtgcctgcaggatacgtctcagaaacatg

SEQ ID NO: 113 - DNA, 17 bp, synthetic construct
  source 1..17: mol_type = other DNA
gtttaagagctaagctg

SEQ ID NO: 114 - DNA, 75 bp, synthetic construct; identical to SEQ ID NO: 82
  source 1..75: mol_type = other DNA

SEQ ID NO: 115 - DNA, 24 bp, synthetic construct
  source 1..24: mol_type = other DNA
cgtgacataaaggagaagctgtgc

SEQ ID NO: 116 - DNA, 27 bp, synthetic construct
  source 1..27: mol_type = other DNA
tgtgctatgttgccctggattttgagc

SEQ ID NO: 117 - DNA, 31 bp, synthetic construct
  source 1..31: mol_type = other DNA
atgttgccctggattttgagcaagaaatggc